SEQUENCE LISTING (88 sequences)

SEQ ID NO: 1 - DNA, 23 bp, Artificial Sequence
  Synthetic
catgcagcttgcggtgagtcctg

SEQ ID NO: 2 - DNA, 23 bp, Artificial Sequence
  Synthetic
gcaatgacacttttctgcttgcc

SEQ ID NO: 3 - DNA, 22 bp, Artificial Sequence
  Synthetic
atgtagagggtcaggaacaccg

SEQ ID NO: 4 - DNA, 23 bp, Artificial Sequence
  Synthetic
agctgatctgaaccactggaaac

SEQ ID NO: 5 - DNA, 22 bp, Artificial Sequence
  Synthetic
tgagacaaccgagacaggaggc

SEQ ID NO: 6 - DNA, 24 bp, Artificial Sequence
  Synthetic
caacaggtttaggaggcagacaag

SEQ ID NO: 7 - DNA, 22 bp, Artificial Sequence
  Synthetic
ctccctctgtgtctcataaacc

SEQ ID NO: 8 - DNA, 21 bp, Artificial Sequence
  Synthetic
gctggcgatgacaagggaggc

SEQ ID NO: 9 - DNA, 23 bp, Artificial Sequence
  Synthetic
ccacaaccatctccattggctgg

SEQ ID NO: 10 - DNA, 22 bp, Artificial Sequence
  Synthetic
ggctgttgggagtacaggagcg

SEQ ID NO: 11 - DNA, 22 bp, Artificial Sequence
  Synthetic
cagatgcagcctatcatggcag

SEQ ID NO: 12 - DNA, 22 bp, Artificial Sequence
  Synthetic
accaggcattggagatggttgg

SEQ ID NO: 13 - DNA, 24 bp, Artificial Sequence
  Synthetic
gccattctccaactctcaaatgtc

SEQ ID NO: 14 - DNA, 25 bp, Artificial Sequence
  Synthetic
agaggctctggtggtgaatataatg

SEQ ID NO: 15 - DNA, 23 bp, Artificial Sequence
  Synthetic
gaagaagaagaagatgaaggtga

SEQ ID NO: 16 - DNA, 23 bp, Artificial Sequence
  Synthetic
gttgttgttattatcatcatcac

SEQ ID NO: 17 - DNA, 23 bp, Artificial Sequence
  Synthetic
gcagcaccttagtaaacaactcc

SEQ ID NO: 18 - DNA, 22 bp, Artificial Sequence
  Synthetic
tccacggcttctatgcgagcac

SEQ ID NO: 19 - DNA, 24 bp, Artificial Sequence
  Synthetic
gagattgaatcatttaatgcgacc

SEQ ID NO: 20 - DNA, 22 bp, Artificial Sequence
  Synthetic
gacagcacctgggctacccggt

SEQ ID NO: 21 - DNA, 23 bp, Artificial Sequence
  Synthetic
gagggagagacccatttgcgttc

SEQ ID NO: 22 - DNA, 23 bp, Artificial Sequence
  Synthetic
gtgtcatccacggcttgtccctc

SEQ ID NO: 23 - DNA, 21 bp, Artificial Sequence
  Synthetic
agatagggaaatggtgcaggt

SEQ ID NO: 24 - DNA, 21 bp, Artificial Sequence
  Synthetic
ctaatggcaattgcagctctc

SEQ ID NO: 25 - DNA, 21 bp, Artificial Sequence
  Synthetic
gatcagcaattatgcacaacg

SEQ ID NO: 26 - DNA, 21 bp, Artificial Sequence
  Synthetic
ccgccaccattcagattatgt

SEQ ID NO: 27 - DNA, 21 bp, Artificial Sequence
  Synthetic
gtggcgttggcagtggtaccg

SEQ ID NO: 28 - DNA, 22 bp, Artificial Sequence
  Synthetic
cagtttgatggtccccattagc

SEQ ID NO: 29 - DNA, 22 bp, Artificial Sequence
  Synthetic
gcatcacagtgcaatttagctg

SEQ ID NO: 30 - DNA, 21 bp, Artificial Sequence
  Synthetic
ccgctgagtttgccttgctgg

SEQ ID NO: 31 - DNA, 23 bp, Artificial Sequence
  Synthetic
caacagcctccttgtctgcctcc

SEQ ID NO: 32 - DNA, 23 bp, Artificial Sequence
  Synthetic
tggtgatgaccagaagggatacc

SEQ ID NO: 33 - DNA, 23 bp, Artificial Sequence
  Synthetic
ccacaaattcttacggaatgtgg

SEQ ID NO: 34 - DNA, 23 bp, Artificial Sequence
  Synthetic
cgcaacaggtttaggaggcagac

SEQ ID NO: 35 - DNA, 22 bp, Artificial Sequence
  Synthetic
ctggcttggctacacttgcatc

SEQ ID NO: 36 - DNA, 22 bp, Artificial Sequence
  Synthetic
cactcactcgtcactagtagcc

SEQ ID NO: 37 - DNA, 24 bp, Artificial Sequence
  Synthetic
gtggttactggtcacccttggcac

SEQ ID NO: 38 - DNA, 25 bp, Artificial Sequence
  Synthetic
gcacaatcttagcattgttgaatgg

SEQ ID NO: 39 - DNA, 24 bp, Artificial Sequence
  Synthetic
catgggttctcatgtgttcatgtg

SEQ ID NO: 40 - DNA, 24 bp, Artificial Sequence
  Synthetic
cgttcaatactccctcaagatcag

SEQ ID NO: 41 - DNA, 21 bp, Artificial Sequence
  Synthetic
acttcaccttctatgcccctg

SEQ ID NO: 42 - DNA, 20 bp, Artificial Sequence
  Synthetic
gttggccaaatcccaagacg

SEQ ID NO: 43 - DNA, 24 bp, Artificial Sequence
  Synthetic
gattggccttacgagtgcgtgaga

SEQ ID NO: 44 - DNA, 24 bp, Artificial Sequence
  Synthetic
aaactctcacgcactcgtaaggcc

SEQ ID NO: 45 - DNA, 24 bp, Artificial Sequence
  Synthetic
gattgctccccgtggtggttctca

SEQ ID NO: 46 - DNA, 24 bp, Artificial Sequence
  Synthetic
aaactgagaaccaccacggggagc

SEQ ID NO: 47 - DNA, 22 bp, Artificial Sequence
  Synthetic
ttctctcgctactcctcctacc

SEQ ID NO: 48 - DNA, 23 bp, Artificial Sequence
  Synthetic
gtactctgcctaagcaacaacca

SEQ ID NO: 49 - DNA, 22 bp, Artificial Sequence
  Synthetic
gagtgagcgagtgagcactgcc

SEQ ID NO: 50 - DNA, 22 bp, Artificial Sequence
  Synthetic
caaattccgcaagcattttgtg

SEQ ID NO: 51 - DNA, 34 bp, Artificial Sequence
  Synthetic
aaaaagcaggctccatgccgcgcccagggccaag

SEQ ID NO: 52 - DNA, 37 bp, Artificial Sequence
  Synthetic
aagaaagctgggtccgaacctggcctactagtaaaac

SEQ ID NO: 53 - DNA, 23 bp, Artificial Sequence
  Synthetic
gagtgagtgagcactgtgttgtg

SEQ ID NO: 54 - DNA, 23 bp, Artificial Sequence
  Synthetic
accaaaaccgccccaagacactc

SEQ ID NO: 55 - DNA, 24 bp, Artificial Sequence
  Synthetic
ggtacggacatagttcacgatccc

SEQ ID NO: 56 - DNA, 25 bp, Artificial Sequence
  Synthetic
gattccttgtccatatccattatcc

SEQ ID NO: 57 - DNA, 32 bp, Artificial Sequence
  Synthetic
ccaagcttggtacggacatagttcacgatccc

SEQ ID NO: 58 - DNA, 32 bp, Artificial Sequence
  Synthetic
gtcctaggcttcgaaatgaaagtgttaaactc

SEQ ID NO: 59 - DNA, 30 bp, Artificial Sequence
  Synthetic
ccaagcttggtacggacatagttcacgatc

SEQ ID NO: 60 - DNA, 31 bp, Artificial Sequence
  Synthetic
tgcggccgccttcgaaatgaaagtgttaaac

SEQ ID NO: 61 - DNA, 20 bp, Artificial Sequence
  Synthetic
tgtctgtctgttggggagag

SEQ ID NO: 62 - DNA, 19 bp, Artificial Sequence
  Synthetic
cactcaattatttctgtcc

SEQ ID NO: 63 - DNA, 20 bp, Artificial Sequence
  Synthetic
gagaacaatatgggaagaag

SEQ ID NO: 64 - DNA, 20 bp, Artificial Sequence
  Synthetic
cttggggcggttttggtttg

SEQ ID NO: 65 - DNA, 20 bp, Artificial Sequence
  Synthetic
tgtctgtctgttggggagag

SEQ ID NO: 66 - DNA, 20 bp, Artificial Sequence
  Synthetic
cttggggcggttttggtttg

SEQ ID NO: 67 - DNA, 1662 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttttttctttttccaagtactgtacccccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccgagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggagaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgcgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccgccactcaattatttctgtccatttcttcttcccatattgttctcgtgtgttgtgtgtgtcagtgttttggtgtgtgtgtgtgtgtgtgtgtgagtgagagaaagagaaaaaaagaaaagaaaaagagtgtcttggggcggttttggtttgagtttaacactttcatttcgaag

SEQ ID NO: 68 - DNA, 1647 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttttttctttttccaagtactgtacccccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccgagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggagaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgcgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccactcaattatttctgtccatttcttcttcccatattgttctcgtgtgttgtgtgtgtcagtgttttggtgtgtgtgtgagtgagagaaagagaaaaaaagaaaagaaaaagagtgtcttggggcggttttggtttgagtttaacactttcatttcgaag

SEQ ID NO: 69 - DNA, 1656 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttttttctttttccaagtactgtacccccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccgagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggagaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgcgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacactcaattatttctgtccatttcttcttcccatattgttctcgtgtgttgtgtgtgtcagtgttttggtgtgtgtgtgtgtgtgtgtgtgagtgagagaaagagaaaaaaagaaaagaaaaagagtgtcttggggcggttttggtttgagtttaacactttcatttcgaag

SEQ ID NO: 70 - DNA, 1576 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttttttctttttccaagtactgtacccccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccgagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggagaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgcgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccgccactcaattatttctgtccatttcttcttcccatattgttctccttggggcggttttggtttgagtttaacactttcatttcgaag

SEQ ID NO: 71 - DNA, 1527 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttttttctttttccaagtactgtacccccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccgagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggagaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgcgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacttggggcggttttggtttgagtttaacactttcatttcgaag

SEQ ID NO: 72 - DNA, 23 bp, Artificial Sequence
  Synthetic
ggccttacgagtgcgtgagaagg

SEQ ID NO: 73 - DNA, 23 bp, Artificial Sequence
  Synthetic
ccttctcacgcactcgtaaggcc

SEQ ID NO: 74 - DNA, 23 bp, Artificial Sequence
  Synthetic
gctccccgtggtggttctcaagg

SEQ ID NO: 75 - DNA, 23 bp, Artificial Sequence
  Synthetic
ccttgagaaccaccacggggagc

SEQ ID NO: 76 - DNA, 13 bp, Glycine max
gacacgccgccac

SEQ ID NO: 77 - DNA, 23 bp, Glycine max
gttttggtgtgtgtgtgtgtgtg

SEQ ID NO: 78 - DNA, 4556 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttttttctttttccaagtactgtacccccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccgagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggagaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgcgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccgccactcaattatttctgtccatttcttcttcccatattgttctcgtgtgttgtgtgtgtcagtgttttggtgtgtgtgtgtgtgtgtgtgtgagtgagagaaagagaaaaaaagaaaagaaaaagagtgtcttggggcggttttggtttgagtttaacactttcatttcgaagatgccgcgcccagggccaaggccttacgagtgcgtgagaagggcttggcacagcgagcgccaccaacccgtgagaggttccatcattcagcagattttcaggttttgggttcttctcgtctcccccccacccccactttttctcgcgcttgtgcattcctctgactctattgcacgccatgtgtttgattagggtcgtcaatgacgctcacagtcccgcaaccaagaagaacaaggaatggcaggagaagctccccgtggtggttctcaaggccgaagaaatcatgtactccaaagccaattccgaggtttttctctcgtttccacaaattcttacggaatgtggtggtggattcttagatctttcactgtgttgggtgaaaccccttttgcttaatatttttgtgatttggttgttgcttaggcagagtacttgaatcctgacacgctctgggaccgcctcaatgatgccgttaacacaatcatacggagagatgagacaaccgagacaggaggcctcttgcccccatgtgttgaaggtacttccattttttcatcttctgtggatccactcttaagagtctgtctcaaaatcctatttttatgtgtcattgtgtggagattagtagctttgagctgccagtgatgtgtttttgtttctttggttttctgggtccattcatttttgtgttgatcacagctgcacttaacttgggatgcaaagccgtgagaacttccagaagtgatcggcacaataacccaaggacttacctttctccaaggattcaacagcctccttgtctgcctcctaaacctgttgcggggaatcccttgaactatgcaaaagtcacaactcctgctgtgtcaccaattcctgtgccagattctatccacccaaatagcaggctcatgggttcctctaagtaccctttctcagaaggtatcccttctggtcatcaccagccattgacaatggaagctagaccctcattgaacttgggttcagtttaccctttgtactatggttatgaagccagagaacctgaaccttggacgactgctacagataccacgtgttctgatacaatctttgttggcagaccagtcatttcagtccctgagccttcaggaattggtctatcggaaaacttctcctatggtacattccatcacgttgcaaacagaatgagaaaagagactgctgttggcactcaggaggcagcaccagatagggaatgtgacttgtctttgaggttgggtcaatgtctgcatccgtgttcaagcagcaaaagtagttcagcatatgaagtagatgatgttggtttaggagtttcaccggaaagctgcaagtttagccatttatccttgcagagaaatagagagttctgtttttatcctagagagacaggttatggcaccattgagtccacttctggtaaatgtaatgtagagggtgaagatctgaatttagaggcaactcttcggaagcgcaaagcacctttatgtggtaacaacgaggaagatgggcaattttgccggaatccaggggtcccatccaatcgttttactagtaggccaggttcgtagacagtttcttgttgtatcatagtatatgcatttgcatttctagatagatgaatgatccaaaatgttgtttaatcctcttatgtagactctttatgtgtagctctgcttgtgaaaaattttagaagctggcgttgagttgctaattgcattttctcccaatgtgaactgtatataaaatcttagtttgctttgcttattttccatttggctctgaaaattaggagggaaaaataaaatgtgaacttctgaaatggggtgtataaaaggaatcatatctatctcctagtaaaagattaaacaagtctaattcaattttgatgcatgtcaaaaatatgacgggtgtgtaatgttaagcctgctacctggaccgagcctgattcataaatcatagtggttcagactctgatgtgttaagctaaaatctcattttttaaaatcaagattagcttctggctttcagcggtgtctattcatgcaaattatcaaagttacatgtcccaaggtgtgtttcaattatgacgttgtcagtgacaaaatagaacaaacttgtgagatattaaataattactgtacggtgattctacaatagtacaatgcaccttccaagaactatatcattgatgcaccagcttctccagccattgactaagctgtttttatgctctgataaatgaaatatcatcctaatagtatgaaggtgcattttagacgccaccaactctagctttctaaaaccttatctgaatcatagttttaaaatccggggcaggaagcaaaggagatggtaatattgtgagaagacatgtcaatactgtgaactggtgaaagttatttttttcttgtcaagggcttgtcaaaattgttaagggtttattgtctgattaggaaagttatgttacattttttaggtgtaagggtgaacaaaagagagaaaattagttaatgtaacaaatgtaatgataatgaaaaatgatgagatgagaagaaaaactgatcatctatttggacaaaaagttgatatggagggattagggcttttgaccaaaatctgaaaagaagctctttcactttgtagaaagaaggaatgagggggatattgttgcctaccataaacaatctgtgcatcaacattaaatacatgctctgcaatgtggatcagaaatgcaattatttctcaagagtgcctttacacctgtcaactattgaactgctcggctaaagatttgctcaagatcctgtgtacttgtcggatccatggaccacatttagcagtctccatgcttggataatggatatggacaaggaatc

SEQ ID NO: 79 - DNA, 4538 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttctttttccaagtactgtaccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccaagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggggaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgtgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcgagaggtgtgaccagaccccacccccctactcacacaaaggagaaaagattgaacactaacatcaaacaacaaacaaaaaagaccatggtaatggtaaaccctctaaattattggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccactcaattatttctgtccatttcttcttcccatattgttctcgtgtgttgtgtgtgtcagtgttttggtgtgtgtgtgagtgagagaaagagaaaaaaagaaaagaaaaagagtgtcttggggcggttttggtttgagtttaacactttcatttcgaagatgccgcgcccagggccaaggccttacgagtgcgtgagaagggcttggcacagcgagcgccaccaacccgtgagaggttccatcattcagcagattttcaggttttgggttcttctcgtctcccccccacccccactttttctcgcgcttgtgcattcctctgactctattgcacgccatgtgtttgattagggtcgtcaatgacgctcacagtcccgcaaccaagaagaacaaggaatggcaggagaagctccccgtggtggttctcaaggccgaagaaatcatgtactccaaagccaattccgaggtttttctctcgtttccacaaattcttacggaatgtggtggtggattcttagatctttcactgtgttgggtgaaaccccttttgcttaatatttttgtgatttggttgttgcttaggcagagtacttgaatcctgacacgctctgggaccgcctcaatgatgccgttaacacaatcatacggagagatgagacaaccgagacaggaggcctcttgcccccatgtgttgaaggtacttccattttttcatcttctgtggatccactcttaagagtctgtctcaaaatcctatttttatgtgtcattgtgtggagattagtagctttgagctgccagtgatgtgtttttgtttctttggttttctgggtccattcatttttgtgttgatcacagctgcacttaacttgggatgcaaagccgtgagaacttccagaagtgatcggcacaataacccaaggacttacctttctccaaggattcaacagcctccttgtctgcctcctaaacctgttgcggggaatcccttgaactatgcaaaagtcacaactcctgctgtgtcaccaattcctgtgccagattctatccacccaaatagcaggctcatgggttcctctaagtaccctttctcagaaggtatcccttctggtcatcaccagccattgacaatggaagctagaccctcattgaacttgggttcagtttaccctttgtactatggttatgaagccagagaacctgaaccttggacgactgctagagataccacgtgttctgatacaatctttgttggcagaccagtcatttcagtccctgagccttcaggaattggtctattggaaaacttctcctatggtagattccatcacgttgcaaacagaatgagaaaagagactgctgttggcactcaggaggcagcactagatagggaatgtgacttgtctttgaggttgggtcaatgtttgcatccgtgttcaagcagcaaaagtagttcagcatatgaagtagatgatgttggtttaggagtttcaccggaaagctgcaagtttagccatttatccttgcagagaaatagagagttctgtttttatcctagagagacaggttatggcaccattgagtccacttctggtaaatgtaatgtagagggtgaagatcagaatttagaggcaactcttcggaagcgcaaagcacctttatgtggtaacaacgaggaagatgggcaattttgccggaatccaggggtcccatccaatcgctttactggtaggccaggttcgtagacagtttcttgttgtatcatagtatatgcatttgcatttctagatagatgaatgatccaaaatgttgtttaatcctcttatgtagactctctttatgtgtagctctgcttgtgaaaaattttagaagctggcgttgagttgctaattgcattttctcccaatgtgaactgtatataaaatcttagtttgctttgcttattttccatttggctctgaaaattaggagggaaaaataaaatgtgaacttctgaaatggggtgtataaaaggaatcatatctatctcctagtaaaagattaaacaagtctaattcaattttgatgcatgtcaaaaatatgacgggtgtgtaatgttaagcctgctacctggaccgagcctgattcataaatcatagtggttcagactctgatgtgttaagctaaaatctctattttttaaaatcaagattagcttctggctttcagcggtgtctattcatgcaaattatcaaagttacatgtcccaaggtgtgtttcaattatgacgttgtcagtgacaaaatagaacaaacttgtgagatattaaataattactgtacggtgattctacaatagtacaatgcaccttccaagaactatatcattgatgcaccagcttctccagccattgactaagctgtttttatgctctgataattgaaatatcatcctaatagtatgaaggtgcattttagacgccaccaactctagcttttctaaaaccatatctgaatcatagttttaaaatccggggcaggaagcaaaggagatggtaatattgtgagaagacatgttaatactgtgaaatggtgaaggttatttttttcttgtcaagggcttgtcaaaattgttaagggtttattgtctgattaggaaagttatgttacattttttaggtgtaagggtgaacaaaagagagaaaattagttaatgtaacaaatgcaatgataatgaaaaatgatgagatgagaagaaaaactgatcatctatttggacaaaaagttgatatggagggattagggcttttgaccaaaatctgaaaagaagctctttcactttgtagaaagaaggaatgagggggatattgttgcctaccataaacaatctgtgcatcaacattaaatacatgctctgcaatgtggatcagaaatgcaattatttctcaagagtgcctttacacctgtcaactattgaactgctcggctaaagatttgctcaagatcctgtgtacttgtcggatccatggaccacatttagcagtctccatgcttggataatggatatggacaaggaatc

SEQ ID NO: 80 - DNA, 4538 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttctttttccaagtactgtaccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccaagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggggaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgtgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctccagaggtgtgaccagaccccacccccctactcacacaaaggagaaaagattgaacactaacatcaaacaacaaacaaaaaagaccatggtaatggtaaaccctctaaattatcggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccactcaattatttctgtccatttcttcttcccatattgttctcgtgtgttgtgtgtgtcagtgttttggtgtgtgtgtgagtgagagaaagagaaaaaaagaaaagaaaaagagtgtcttgggacggttttggtttgagtttaacactttcatttcgaagatgccgcgcccagggccaaggccttacgagtgcgtgagaagggcttggcacagcgagcgccaccaacccgtgagaggttccatcattcagcagattttcaggttttgggttcttctcgtctcccccccacccccactttttctcgcgcttgtgcattcctctgactctattgcacgccatgtgtttgattagggtcgtcaatgacgctcacagtcccgcaaccaagaagaacaaggaatggcaggagaagctccccgtggtggttctcaaggccgaagaaatcatgtactccaaagccaattccgaggtttttctctcgtttccacaaattcttacggaatgtggtggtggattcttagatctttcactgtgttgggtgaaaccccttttgcttaatatttttgtgatttggttgttgcttaggcagagtacttgaatcctgacacgctctgggaccgcctcaatgatgccgttaacacaatcatacggagagatgagacaaccgagacaggaggcctcttgcccccatgtgttgaaggtacttccattttttcatcttctgtggatccactcttaagagtctgtctcaaaatcctatttttatgtgtcattgtgtggagattagtagctttgagctgccagtgatgtgtttttgtttctttggttttctgggtccattcatttttgtgttgatcacagctgcacttaacttgggatgcaaagccgtgagaacttccagaagtgatcggcacaataacccaaggtcttacctttctccaaggattcaacagcctccttgtctgcctcctaaacctgttgcggggaatcccttgaactatgcaaaagtcacaactcctgctgtgtcaccaattcctgtgccagattctatccccccaaatagcaggctcatgggttcctctaagtaccctttctcagaaggtatcccttctggtcatcaccagccattgacaatggaagctagaccctcattgaacttgggttcagtttaccctttgtactatgattatgaagccagagaacctgaaccttggacgactgctagagataccacgtgttctgatacaatctttgttggcagaccagtcatttcagtccctgagccttcaggaattggtctattggaaaacttctcctatggtagattccatcacgttgcaaacagaatgagaaaagagactgctgttggcactcaggaggcagcactagatagggaatgtgacttgtctttgaggttgggtcaatgtttgcatccatgttcaagcagcaaaagtagttcagcatatgaagtagatgatgttggtttaggagtttcaccggaaagctgcaagtttagccatttatccttgcagagaaatagagagttctgtttttatcctagagagacaggttatggcaccattgagtccacttctggtaaatgtaatgtagagggtgaagatcagaatttagaggcaactcttcggaagcgcaaagcacctttatgtggtaacaacgaggaagatgggcaattttgccggaatccaggggtcccatccaatcgctttactggtaggccaggttcgtagacagtttcttgttgtatcatagtatatgcatttgcatttctagatagatgaatgatccaaaatgttgtttaatcctcttatgtagactctctttatgtgtagctctgcttgtgaaaaattttagaagctggcgttgagttgctaattgcattttctcccaatgtgaactgtatataaaatcttagtttgctttgcttattttccatttggctctgaaaattaggagggaaaaataaaatgtgaacttctgaaatggggtgtataaaaggaatcatatctatctcctagtaaaagattaaacaagtctaattcaattttgatgcatgtcaaaaatatgacgggtgtgtaatgttaagcctgctacctggaccgagcctgattcataaatcatagtggttcagactctgatgtgttaagctaaaatctctattttttaaaatcaagattagcttctggctttcagcggtgtctattcatgcaaattatcaaagttacatgtcccaaggtgtgtttcaattatgacgttgtcagtgacaaaatagaacaaacttgtgagatattaaataattactgtacggtgattctacaatagtacaatgcaccttccaagaactatatcattgatgcaccagcttctccagccattgactaagctgtttttatgctctgataattgaaatatcatcctaatagtatgaaggtgcattttagacgccaccaactctagcttttctaaaaccatatctgaatcatagttttaaaatccggggcaggaagcaaaggagatggtaatattgtgagaagacatgttaatactgtgaaatggtgaaggttatttttttcttgtcaagggcttgtcaaaattgttaagggtttattgtctgattaggaaagttatgttacattttttaggtgtaagggtgaacaaaagagagaaaattagttaatgtaacaaatgcaatgataatgaaaaatgatgagatgagaagaaaaactgatcatctatttggacaaaaagttgatatggagggattagggcttttgaccaaaatctgaaaagaagctctttcactttgtagaaagaaggaatgagggggatattgttgcctaccataaacaatctgtgcatcaacattaaatacatgctctgcaatgtggatcagaaatgcaattatttctcaagagtgcctttacacctgtcaactattgaactgctcggctaaagatttgctcaagatcctgtgtacttgtcggatccatggaccacatttagcagtctccatgcttggataatggatatggacaaggaatc

SEQ ID NO: 81 - DNA, 4538 bp, Glycine max
ggtacggacatagttcacgatcccttccaccatatctgttttaattttatttattatttttgaagtttgtcttgtaatataaattgaaatgtgttgtactttggagtttaaggagcatcaagtatcaagtagaaacatggaagggtgaggaggagattattcacccactgttgaaacattaggctttttttttttttctttttccaagtactgtaccccccccccaaaggaaaaaagatagaagttggaagtgggatgctcgaggcttacataaaaactgaaaaaggagtttggctttgtgcagccgttgtcaagagtggacgtgacatgagagagtggggaaggcccatacagcgtcagtgttatgccaaaggctgttttcatcacataaagggaattgtttacagcgccaagggtgcagaatgtacccgtttacccatcaatgtgttcactgcacattcaatgcacaatataaatatatatatcttcataagtcataaataaatcaattttacttactagacactagacacgggtcaaggtgactcagataacgtagtcatagacattaatttctacaagggctgacccaccaacataacaattgagagagcaccatgtgcacgttgtaggtggcaacaagagcctagcacaaccgaaataacgatgtttgtccccggaataaaagcatgagtcacggtgaaacaaaaattaaccctaagggtaaaataaaaaaataaaaaaaggggaaagaatctgagtagctttattaaggaagaataagggtgggagggaaactgatgtaaccggattctcttgggctaactttataacgaagttgatattagttatgttttgtcattttccttggcagtgttcatgctggttgaagggttatgtgtacgaatatgctggcattggagatggatgtgcaattatgtaaaatagataataatgcattgcatggtaatgtaatgttatgtaacaacaacaaccaaccctatattaaatatgggtgagggcatggcattgggaaggggagaattttaatgggactttattatacaggtactttttgatagtaacctaactctcccactctccttttctcaagaggtgtgaccagaccccacccccctactcacacaaaggagaaaagattgaacactaacatcaaacaacaaacaaaaaagaccatggtaatggtaaaccctctaaattatcggggaagacaaaagaatgagaaagaaagctgccctttctctcgctactcctcctaccccaaacaaaactttcccattacaaattgtggaatgaagaaagaatggcaagacaagtgagtagtagtatagtagtaattgacccatcaaacaaacaagcataacccatgtaattgttattgtagatagatagagtgagtgagtgagcactgtgttgtgtatatagtagtgtgtaaactctccccaacagacagacacgccactcaattatttctgtccatttcttcttcccatattgttctcgtgtgttgtgtgtgtcagtgttttggtgtgtgtgtgagtgagagaaagagaaaaaaagaaaagaaaaagagtgtcttgggacggttttggtttgagtttaacactttcatttcgaagatgccgcgcccagggccaaggccttacgagtgcgtgagaagggcttggcacagcgagcgccaccaacccgtgagaggttccatcattcagcagattttcaggttttgggttcttctcgtctcccccccacccccactttttctcgcgcttgtgcattcctctgactctattgcacgccatgtgtttgattagggtcgtcaatgacgctcacagtcccgcaaccaagaagaacaaggaatggcaggagaagctccccgtggtggttctcaaggccgaagaaatcatgtactccaaagccaattccgaggtttttctctcgtttccacaaattcttacggaatgtggtggtggattcttagatctttcactgtgttgggtgaaaccccttttgcttaatatttttgtgatttggttgttgcttaggcagagtacttgaatcctgacacgctctgggaccgcctcaatgatgccgttaacacaatcatacggagagatgagacaaccgagacaggaggcctcttgcccccatgtgttgaaggtacttccattttttcatcttctgtggatccactcttaagagtctgtctcaaaatcctatttttatgtgtcattgtgtggagattagtagctttgagctgccagtgatgtgtttttgtttctttggttttctgggtccattcatttttgtgttgatcacagctgcacttaacttgggatgcaaagccgtgagaacttccagaagtgatcggcacaataacccaaggacttacctttctccaaggattcaacagcctccttgtctgcctcctaaacctgttgcggggaatcccttgaactatgcaaaagtcacaactcctgctgtgtcaccaattcctgtgccagattctatccacccaaatagcaggctcatgggttcctctaagtaccctttctcagaaggtatcccttctggtcatcaccagccattgacaatggaagctagaccctcattgaacttgggttcagtttaccctttgtactatgattatgaagccagagaacctgaaccttggacgactgctagagataccacgtgttctgatacaatctttgttggcagaccagtcatttcagtccctgagccttcaggaattggtctattggaaaacttctcctatggtagattccatcacgttgcaaacagaatgagaaaagagactgctgttggcactcaggaggcagcactagatagggaatgtgacttgtctttgaggttgggtcaatgtttgcatccatgttcaagcagcaaaagtagttcagcatatgaagtagatgatgttggtttaggagtttcaccggaaagctgcaagtttagccatttatccttgcagagaaatagagagttctgtttttatcctagagagacaggttatggcaccattgagtccacttctggtaaatgtaatgtagagggtgaagatcagaatttagaggcaactcttcggaagcgcaaagcacctttatgtggtaacaacgaggaagatgggcaattttgccggaatccaggggtcccatccaatcgctttactggtaggccaggttcgtagacagtttcttgttgtatcatagtatatgcatttgcatttctagatagatgaatgatccaaaatgttgtttaatcctcttatgtagactctctttatgtgtagctctgcttgtgaaaaattttagaagctggcgttgagttgctaattgcattttctcccaatgtgaactgtatataaaatcttagtttgctttgcttattttccatttggctctgaaaattaggagggaaaaataaaatgtgaacttctgaaatggggtgtataaaaggaatcatatctatctcctagtaaaagattaaacaagtctaattcaattttgatgcatgtcaaaaatatgacgggtgtgtaatgttaagcctgctacctggaccgagcctgattcataaatcatagtggttcagactctgatgtgttaagctaaaatctctattttttaaaatcaagattagcttctggctttcagcggtgtctattcatgcaaattatcaaagttacatgtcccaaggtgtgtttcaattatgacgttgtcagtgacaaaatagaacaaacttgtgagatattaaataattactgtacggtgattctacaatagtacaatgcaccttccaagaactatatcattgatgcaccagcttctccagccattgactaagctgtttttatgctctgataattgaaatatcatcctaatagtatgaaggtgcattttagacgccaccaactctagcttttctaaaaccatatctgaatcatagttttaaaatccggggcaggaagcaaaggagatggtaatattgtgagaagacatgttaatactgtgaaatggtgaaggttatttttttcttgtcaagggcttgtcaaaattgttaagggtttattgtctgattaggaaagttatgttacattttttaggtgtaagggtgaacaaaagagagaaaattagttaatgtaacaaatgcaatgataatgaaaaatgatgagatgagaagaaaaactgatcatctatttggacaaaaagttgatatggagggattagggcttttgaccaaaatctgaaaagaagctctttcactttgtagaaagaaggaatgagggggatattgttgcctaccataaacaatctgtgcatcaacattaaatacatgctctgcaatgtggatcagaaatgcaattatttctcaagagtgcctttacacctgtcaactattgaactgctcggctaaagatttgctcaagatcctgtgtacttgtcggatccatggaccacatttagcagtctccatgcttggataatggatatggacaaggaatc

SEQ ID NO: 82 - DNA, 3409 bp, Glycine max
tttaacactttcatttcgaagatgccacgcccagggccaaggccttacgagtgcgtgagaagggcttggcacagcgagcgccaccaacccgtgagaggttccatcattcagcagattttcaggttttgggttcttctcgttttccccctctcttttttctatctctcgcccctttttcgctcttgtgcattcccctgacgctattgcacgccatgtgtttgattagggtcgtcaatgacgctcacagtcccgcgaccaagaagaacaaggaatggcaagagaagctccccgtggtggttctcaaggccgaagaaatcatgtactccaaagccaattccgaggttttttctcctttcacaaaatgcttgcggaatttgaattcttagatcttttcactgtgttgggtgaaaccccttttgcttaatttttttgtgatttggttgcttaggcagagtacttgaatcctgatacgctctgggaccgcctcaatgatgccgttaacacaatcatacggagagatgagaccaccgagacaggagacctcttgcccccatgtgttgaaggtacttcattttttcatcttctgtggatccactcttaagtgtctgtctcaaaatcctatttttatgtgtcattgtgtggagattagttgctttgagctgccagtgatgtgtttctgtttttatggttttctgggtccattcattgggttgatcacagctgcacttaacctgggatgcaaacctgtgagaacttccagaagtgatcggcacaataacccaaggacttacctttcttcaaggattcaacaacctccctctgtgtctcataaacctgatgctgggaatcccttgaactatgccaaagtcacaactcctgctgtctcaccaattcctgtgccagattctacccaccaaaatagcaagctcatgggttcctctaactaccctttctcagaaggcctcccttgtcatcgccagccattgacaaaggaagctagaccctcattgaacttgggttcagtttacccattgtactatggttatgaagccagagaacctcaacctaggacgactgctagagataccacgtgttctgatacaatctttgttggcagaccagtcattccagtccctgagccttcaggaattggtctattggaaaacttctcctatggtagattccatcacgttgcaaacagaataggaaaagagattgctgttggcactcaggaggcagcaccagatagggaatgtgacttgtctttgaggttgggtcaatgtctgcatccgtgttcaagcagcaaaagtagttcagcgtatgaagtagatgatgttggtttaggagtttcaccgggaagcagcaggtttagccatttatccttgcagaaatatagggagttctgtttttatcctagagagacaggttatggcaccattgagtccacttctggtaaatgtaatgtagagggtgaagatcagaacttagaggcaactcttaggaagcgcaaagcacctttaggtaacaacgaagaagatgggcaattttgccggaatctaggggtcccatccaatcgttttactggtaggccaggttcgtagacagcttattgttgtatcatgtatgcatttgcctttctagatgaatgatccaaaatgttgtttaatcctcttgtgtagactctatgtgtagctctgcttgtgaaaaaatttagaagctggcgttgagttgctaattacatttctgccaatgtgaactgtatataaaatcttagcttgctttgcttattttcgactaggctctgaaaaaattagaagagggaaaaatgaaatggggtgtataaaaatcatatctatctcctagtaaaagattaaagaagtctaattcaattttgatgcatggaccgggcctgattcataaaatcgtagtggcgcagactctaatgtgttatagtagctaaaatctctatttttagtcaagattagcttctggctttcagcggtgtctatttatgcaaattatcaaagttgcatgtcccaaacaaaatagaacaaacttgtgacattaaataattactgcacggtggttctacaatagtacaatgcaccctgcatgaactttagcatggatgccccagctcctccagccattgactaagctgtttttatggtcgataattgaaatatcatcctaatctaatggtggagttagaaggtgcattttagactccaccaactaaacaacatagactctagctccttaaaaccacatccgaatcatagttttaatatccaaggcaggaaacaaaggagatgataatattgtgagaagacatgttaataccgtgaaatggtggaagttatgattttcttgtcaaaggcatctcaaaattgataagggtttgttgctctgataaggaaaatgtttaatgtatgtttcaatttttagatgcaaaggtgaacaaaagagagaaattagtgaatgtaacaatgttataatgaaaatgaaaaatgatgagatgagaataaaatgaggtaaacgagagagggaagaaaaactgattatctgtttggacaaaaagctgatatggagggattaggattagggatttgaccaatctcaaaaaaagctctttcacttagtagatagatggaatgaggtgggatattgttgcctacataaaccatctgtgcatcaacattaaatacatgctctgcaatgtggatcagaaatgcaattatttcttaagagtgcctttacacctgtcaactattgaactgctcggctagaagatttgcccaagatcctgtgtacttgtcggatccatggaccacatttagccgtctccatgctttgatatggacaaggaatcagtttgaagtctatccgttcaagtgcattagggagtgactgagtgagtttcaaaataataatataataaacaacaactttgagagatgtcttttgggatgattagcagaatttcctgccgtacccggggccgatttacaattggcttttgctctccttacattttggtgaatggacgtctaaaccgctaattaatatagtcttctagcacaaaactcttgactttttgtttggaaaagttatgttttgttaattctaattattatttgacttatgggatcatattaaaaatccatttacacgagtgcatggctcaaaattatattaacttatctaaatcgtgtttaaggattgagaatggaagttgtgttaaatatttagtgaaacagatttattgcatatagtagtcttttattcaatccaaacaaaattgctttttacatatgatacatatggttctgttaaatgataaacttacagtaacttttgttgtacctgtaaagtaattatttaacggatgtgacatgcgtctaaaggg

SEQ ID NO: 83 - DNA, 3275 bp, Glycine max
tccttagtcctttctttcctcctcctactacacgaccaaaactatgccattacaggcaagacctaaagtttgaagaagcacagcgcaccaaaacctaggtagagagaatcaactagtgtactacagtactagctagctcctctcaggtcctaacacaagatagagacaaaaaaaaaaacgccacaaatcacaaaacacaaaaaaaatcagttttttcttgaagatgccgaggcccggccctagaccttacgagtgcatgaggcgagcttggcacagcgagcggcaccaacccatgagaggttccatcattcggcaaattttcaggtttattttttatttttttctctccttttattcaatgctctgttcattttacatctggatagttcagttggtttaagagtgtgcgtgagttattgaaaatcctctgaccgagcgtgcgtgacgcgttcaattagggtcgtcgcggaggctcactccgcggctactaagagcaacaaggagtggcaagagaagctccccatagttgtcctcagagccgaggaaatcatctactccaaagccaattccgaggttgttgttagttctctctgattccaccaaaatgcatgtcacagattctttagatctttgtcgtcaaaaaaaaaaaaaacctcgtcaattttggtttccctttgcttaggcagagtacctcaaccccgacacgctcttggaccgactcaatgatgccatcaacaccatcatacgcagagacgaaaccaccgagaccggtcagcttttgccgccatgcgttgaaggtactgaaaattagttccaccttatttcaacatttcccttaaaaattttaaaaatttggcgtgtcatttaggaattttagaaggataaatgaccattttagtccttgaatgtatataaagaggttataatttagtttttgaaagaacgaaattctgatttagttctcaaatgtttaaaaatgtttatggatcaatttgtgattaagttgcatatgacaggatgtaattgtctcacttttgatacatttaggactgaatcaaaattttcgttcttttagggactaaattgtcacgggtttatacattcaatatatataaacaggtgacaatttagtttctgaaagatccaaattctgatttagctctcaaatgtttatggattaatttgtctttaagttgtatatgacaggacctaatagtctcacttttgaaacattttggaccgaattagaattttcatcctttcgaagactaaattgtcaccggtttgtagattcaaagaccagaatggtcatttagcaattttaaaacgtgagccatttatgtttttttctttcttcattttgttagctgcacttaatattggttgcaagccagtgagaacatcaagaagtgataggcataaccccagggcttaccttacctccagacatcaactacatccttgttctatttctcccaaaactgttgccaacaatgctttaaactttcttccagtgtcggattctgatgatcacaccaacaaaaataactattcctcattagacactttggcttctgttgttcttcatcaccaacccttggcagtggaaactaacccctcattgagcctgggttcggtttacccattatattatggttttgaagtcaaagagcctcaactaaggaccagcatccgaagaagtacttgttcggatacaatatttgttggaagaccggttattagtccagccttggagccatgtagaatggatctattagaaaacttgtcctgtggtagagtctatcatgctccaaaaagaattatgaaagaaaatgctgtaagcgttgcaggtgagtcgcaagatggggaatgtgatttgtctttgaggttgggtatatgtttaaagagcaacaaagcaagttcagcatatggatcagaagatattgggttaagagtgtctgaagagggcaggaaatttggccattcgtctcgacagaaaaatgaagggtactgtttttaccctagaggaacaggttatggttccatttagtgcatgtttgggaaccagatgtttttgggaagtgatcgttttttaagttctcaataaaaataatacaaataaatgattatttcttcaaaataagctgaacctaacatgcaattattccatgatgttgaagatgatcaaaatgctggcaacttttcagaagtacagtgtgcttctaggtagctaggtggatgggcaattttgatggccatgacggttctgcccattctgtttactggtagaattactgggtcaggtttgtagtttgtctttcatgttgtagcatatttgcatttgcaattgaagttggtccatgatgtttcatcctctcatgtggattctttgtttgtagctatgcttgtaaaaatttaggtggtgccattgagttgctctttgcttggctgcaaaattgagctgtatataaaatcttagcttgctttgttttgtttctacttggctcagaatagatcaaattggagaaagagaaatgaaacaaatgtcttgaatgaatgttcatatgccccaacttaatgatgtctggcctgattcaattcaactacaaaagttttatgtgtggcaaacatataactgatgtactacattatttctgctttctgaaccagtctaaattcatgtggccaggagttcgatctgctaactggaatctcttctcagttacgattagctcccttaacccaatctttgttttgcagaattatgtctaatgcaaatgatcaatcgagagagagaggccataaggtgaattttccattacagtggtcaaatgcaaatgcaattatgtaacttttttaaaaaagaccctgcgtattaaatcactgaacaggacttaatttagatgtttcgcaacatagaactggtgttttaagagatttagtcttgctctttcgtacacttgaccacattaaaaatcagaatgcccaatgttatggacaaggtctaagtttgaagtcaatcaatctgatcctgctattagaagcgtgagtttctaaataataaaaggataactatcttcgagatctctcattattgttgcttttcaaatttcctactgtagcgtgatgaaaattgtttgaaggcaattttggttcactatttctctctatagatttagaattgtgcaattttgatcttttaaatttttttattgcacatgtagttttttatgtttttaaaatttagcaatttttagtgtgttgttagtttgtca

SEQ ID NO: 84 - protein, 422 aa, Glycine max
MPRPGPRPYECVRRAWHSERHQPVRGSIIQQIFRVVNDAHSPATKKNKEWQEKLPVVVLKAEEIMYSKANSEAEYLNPDTLWDRLNDAVNTIIRRDETTETGGLLPPCVEAALNLGCKAVRTSRSDRHNNPRTYLSPRIQQPPCLPPKPVAGNPLNYAKVTTPAVSPIPVPDSIHPNSRLMGSSKYPFSEGIPSGHHQPLTMEARPSLNLGSVYPLYYGYEAREPEPWTTATDTTCSDTIFVGRPVISVPEPSGIGLSENFSYGTFHHVANRMRKETAVGTQEAAPDRECDLSLRLGQCLHPCSSSKSSSAYEVDDVGLGVSPESCKFSHLSLQRNREFCFYPRETGYGTIESTSGKCNVEGEDLNLEATLRKRKAPLCGNNEEDGQFCRNPGVPSNRFTSRPERRNEGDIVAYHKQSVHQH

SEQ ID NO: 85 - protein, 403 aa, Glycine max
MPRPGPRPYECVRRAWHSERHQPVRGSIIQQIFRVVNDAHSPATKKNKEWQEKLPVVVLKAEEIMYSKANSEAEYLNPDTLWDRLNDAVNTIIRRDETTETGDLLPPCVEAALNLGCKPVRTSRSDRHNNPRTYLSSRIQQPPSVSHKPDAGNPLNYAKVTTPAVSPIPVPDSTHQNSKLMGSSNYPFSEGLPCHRQPLTKEARPSLNLGSVYPLYYGYEAREPQPRTTARDTTCSDTIFVGRPVIPVPEPSGIGLLENFSYGRFHHVANRIGKEIAVGTQEAAPDRECDLSLRLGQCLHPCSSSKSSSAYEVDDVGLGVSPGSSRFSHLSLQKYREFCFYPRETGYGTIESTSGKCNVEGEDQNLEATLRKRKAPLGNNEEDGQFCRNLGVPSNRFTGRPGS

SEQ ID NO: 86 - protein, 337 aa, Glycine max
MPRPGPRPYECMRRAWHSERHQPMRGSIIRQIFRVVAEAHSAATKSNKEWQEKLPIVVLRAEEIIYSKANSEAEYLNPDTLLDRLNDAINTIIRRDETTETGQLLPPCVEAALNIGCKPVRTSRSDRHNPRAYLTSRHQLHPCSISPKTVANNALNFLPVSDSDDHTNKNNYSSLDTLASVVLHHQPLAVETNPSLSLGSVYPLYYGFEVKEPQLRTSIRRSTCSDTIFVGRPVISPALEPCRMDLLENLSCGRVYHAPKRIMKENAVSVAGESQDGECDLSLRLGICLKSNKASSAYGSEDIGLRVSEEGRKFGHSSRQKNEGYCFYPRGTGYGSI

SEQ ID NO: 87 - DNA, 20 bp, Artificial Sequence
  Synthetic
ggccttacgagtgcgtgaga

SEQ ID NO: 88 - DNA, 20 bp, Artificial Sequence
  Synthetic
gctccccgtggtggttctca

(19) United States
(12) Patent Application Publication
(10) Pub. No.: US 2022/0154202 A1
(43) Pub. Date: May 19, 2022

(54) Gene Regulating Seed Weight in Improving Seed Yield in Soybean

(71) Applicant: The Curators of the University of Missouri, Columbia, MO (US)

(72) Inventors: Minviluz Stacey, Columbia, MO (US); Cuong X. Nguyen, Columbia, MO (US); Gary Stacey, Columbia, MO (US)

(21) Appl. No.: 17/530,832

(22) Filed: Nov. 19, 2021

Related U.S. Application Data
(60) Provisional application No. 63/115,711, filed on Nov. 19, 2020.

(51) Int. Cl. C12N 15/82

(52) U.S. Cl. CPC C12N 15/8262

(57) ABSTRACT

Provided herein are methods of obtaining, producing, identifying, and the like soybean plants having a genotype associated with a large-seed phenotype as well as plants, plant cells, and plant genomes comprising a genotype associated with a large-seed phenotype.

### CROSS REFERENCE TO RELATED APPLICATIONS

This application claims the benefit of U.S. Provisional Application 63/115,711, filed Nov. 19, 2020, which is incorporated by reference herein in its entirety.

### STATEMENT REGARDING FEDERALLY-SPONSORED RESEARCH AND DEVELOPMENT

This invention was made with government support under grant number 1444581 awarded by the National Science Foundation. The government has certain rights in the invention.

### REFERENCE TO SEQUENCE LISTING SUBMITTED ELECTRONICALLY

The content of the electronically submitted sequence listing in ASCII text file (Name 21UMC0042_212669_SeqList_ST25.txt; Size: 67754 bytes; and Date of Creation: Nov. 19, 2021) filed with the application is incorporated herein by reference in its entirety.

### BACKGROUND

This field of this invention is the use of genetic screening and molecular breeding techniques to identify, obtain, and produce soybean plants, seeds, etc., comprising in their genomes a genetic locus associated with the large-seed phenotype and also towards the use of genetic editing of the soybean genome to produce plants, seeds, etc., having a large-seed phenotype.

Soybean (Glycine max) is one of the most important crops grown world-wide, providing 70% and 28% of the protein meal and vegetable oil consumed, respectively (from the world wide web at soystats.com). Like other staple crop plants, the economic value of soybean is dependent on the quantity (yield) and quality of seeds. Seed yield is determined by two components: the number of seeds produced and seed size (or weight). Besides yield, seed weight is also positively correlated with seed germination, viability and vigor (Edwards & Hartwig, 1971; Burns et al., 1973; Smith & Camper, 1975; Hopper et al., 1979). Increased seed weight thus confers an evolutionary advantage and is likely one of the first traits that was selected during crop domestication (Liu et al., 2007; Purugganan & Fuller, 2009; Zhou et al., 2015). In soybean, seed weight is also a critical trait in selecting cultivars for various soy food products such as sprouts, edamame, soy nuts, natto and miso (Kato et al., 2014). Seed weight is a complex trait controlled by many genetic and environmental factors. Recent studies showed that the heritability of seed weight in soybean is up to 98%, indicating that genetics is the major factor in controlling phenotypic variation of soybean seed weight (Zhang et al., 2016; Yan et al., 2017). Thus, understanding the genetic factors controlling this trait in soybean is critical to current efforts to improve yield potential and soy food.

The final size of plant organs is coordinately controlled by cell proliferation and cell expansion. Organ growth is thus regulated by the proliferation rate and timing of proliferation arrest, which determines the final cell number, and the rate and duration of subsequent cell expansion, which determines the final cell size (Hepworth & Lenhard, 2014). The genetic and molecular mechanisms involved in controlling seed size have been mostly characterized in Arabidopsis and rice and include the ubiquitin-proteasome, G-protein, mitogen-activated protein kinase (MAPK) and phytohormone signaling pathways (Li & Li, 2016; Li et al., 2019). Several transcriptional regulatory factors have also been identified that control cell proliferation and/or cell expansion (Li & Li, 2016; Li et al., 2018b, 2019). Recently, a repressor complex consisting of PEAPOD2 (PPD2), KINASE-INDUCIBLE DOMAIN INTERACTING 8/9 (KIX8/9), and TOPLESS (TPL) proteins (PPD/KIX/TPL complex) was demonstrated to control meristemoid proliferation in Arabidopsis, in part, by negatively regulating the expression of D3-type cyclins (Baekelandt et al., 2018). Arabidopsis plants harboring null mutations in atppd or atkix8/9 showed increased cell proliferation and produced larger leaves and seeds (White, 2006; Gon-zalez et al., 2015; Wang et al., 2016; Baekelandt et al., 2018; Li et al., 2018a). Increased organ size due to downregulation or loss-of-function of putative AtPPD or AtKIX orthologs was also observed in tomato (Swinnen et al., 2020) and in legume plants such as Medicago truncatula (Ge et al., 2016), pea (Pisum sativum) (X. Li et al., 2018), Vigna mungo (Naito et al., 2017) and soybean (Ge et al., 2016; Kanazashi et al., 2018). Recent studies in Arabidopsis identified additional AtPPD2-interacting proteins such as the NOVEL INTERACTOR OF JAZ (NINJA), a transcriptional repressor involved in the jasmonic acid (JA) signaling pathway, and LIKE HETEROCHROMATIN PROTEIN1 (LHP1), a component of Polycomb Repressive Complex 1 (PRC1) (Zhu et al., 2020). More recently, AtKIX8/9 and AtPPD1/2 were also shown to interact with transcription factors AtMYC3/4 to form the KIX-PPD-MYC complex to repress the expression of GROWTH-REGULATING FACTOR (GRF)-INTERACTING FACTOR 1 (GIF1) (Liu et al., 2020), a transcriptional co-activator involved in the regulation of cell proliferation in plants (Kim & Kende, 2004; Horiguchi et al., 2005; Lee et al., 2009; Liu et al., 2020). AtPPD and AtKIX8/9 orthologues are found in lycophytes, eudicots and the monocot species Musa acuminata (banana) and Elaeis guineensis (oil palm), but are absent in grasses (Gonzalez et al., 2015; Zhu et al., 2020).

Seed weight in soybean is a quantitative trait that is governed by multiple genes and can vary to a large degree depending on the cultivar. For example, seed weight varied from 7.3 g to 23.6 g and from 5.64 g to 34.8 g per 100 seeds in U.S. and Chinese germplasm collections, respectively (Zhang et al., 2016; Zhao et al., 2019). A large number of quantitative trait loci (QTLs) associated with seed weight in soybean have been identified, using linkage analysis and genome-wide association studies (GWAS), and documented in SoyBase (located on the world wide web at soybase.org) (Grant et al., 2009). However, the genes underlying these loci remain largely unknown due to the complex soybean genome structure and the low genetic diversity in domesticated soybean populations (Hyten et al., 2006; Schmutz et al., 2010), making high resolution mapping laborious and costly. It is believed that only the Phosphatase 2C (PP2C-1) gene, specifically the PP2C-1 allele of this gene, was shown to contribute to seed weight in soybean using linkage analysis. This gene was identified by whole-genome sequencing of a core set of 198 recombinant inbred lines (RILs) and construction of high-density map (Lu et al., 2017). PP2C was suggested to positively regulate seed size through the brassinosteroid signaling pathway (Jiang et al., 2013; Lu et al., 2017). Reverse genetics approaches were also employed to determine whether orthologous genes with known functions in controlling seed size in Arabidopsis are also operable in soybean. For example, overexpression of GmCYP78A72, an ortholog of AtKLU encoding a cytochrome P450 protein, resulted in increased soybean seed weight (Adamski et al., 2009; Zhang et al., 2015, 2016). Several CYP family members are implicated in controlling seed weight in plants (Li et al., 2019), but so far the mechanism(s) by which they function is (are) unknown.

Thus, there remains a need to identify and manipulate genes associated with seed weight in soybean to improve yield.

### SUMMARY

Seed weight is one of the most important agronomic traits in soybean for yield improvement and food production. Several quantitative trait loci (QTLs) associated with the trait have been identified in soybean. However, the genes underlying the QTLs and their functions remain largely unknown. Using forward genetic methods and CRISPR/Cas9 gene editing, the role of GmKIX8-1 in the control of organ size in soybean was identified and characterized. GmKIX8-1 belongs to a family of KIX domain-containing proteins that negatively regulate cell proliferation in plants. Consistent with this predicted function, it was discovered that loss-of-function GmKIX8-1 mutants showed a significant increase in the size of aerial plant organs, such as seeds and leaves. Likewise, the increase in organ size is due to increased cell proliferation, rather than cell expansion, and increased expression of CYCLIN D3;1-10. Molecular analysis of soybean germplasms harboring the qSw17-1 QTL for the big-seeded phenotype indicated that reduced expression of GmKIX8-1 is the genetic basis of the qSw17-1 phenotype.

Provided for herein is are methods for obtaining a soybean plant comprising in its genome at least one genetic locus that comprises a genotype associated with a large-seed phenotype, the method comprising the steps of (a) genotyping one or more soybean plants with respect to a genetic locus comprising a soybean GmKIX gene; and (b) selecting based on said genotyping of said genetic locus a soybean plant comprising a genotype associated with a large-seed phenotype.

Also provided for herein are methods for producing a soybean plant comprising in its genome an introgressed genetic locus comprising a genotype associated with a large-seed phenotype, the method comprising the steps of (a) crossing a first soybean plant with a genotype associated with a large-seed phenotype in a first polymorphic genetic locus comprising a soybean GmKIX gene with a second soybean plant comprising a genotype not associated with a large-seed phenotype in the polymorphic genetic locus comprising said GmKIX gene and at least one second polymorphic locus that is linked to the genetic locus comprising said GmKIX gene and that is not present in said first soybean plant to obtain a population segregating for the large-seed phenotype polymorphic locus and said linked second polymorphic locus; (b) genotyping for the presence of at least two polymorphic nucleic acids in at least one soybean plant from said population, wherein a first polymorphic nucleic acid is located in said genetic locus comprising said GmKIX gene and wherein a second polymorphic amino acid is the linked second polymorphic locus not present in said first soybean plant; and (c) selecting a soybean plant comprising a genotype associated with the large-seed phenotype and the at least one linked marker found in said second soybean plant that does not comprise a large-seed phenotype locus but not found in said first soybean plant, thereby obtaining a soybean plant comprising in its genome an introgressed large-seed phenotype locus.

Also provides for herein is a soybean plant comprising an introgressed genetic locus comprising a genotype associated with a large-seed phenotype in a genomic region comprising a soybean GmKIX gene, wherein at least one marker linked to the introgressed large-seed phenotype genetic locus found in said soybean plant is characteristic of germplasm comprising a non-large-seed genetic locus but is not associated with germplasm comprising the large-seed phenotype genetic locus.

Also provided for herein are methods of identifying a soybean plant that comprises a genotype associated with a large-seed phenotype, the method comprising (a) genotyping a soybean plant in at least one polymorphic genetic locus associated with a large-seed phenotype for the presence of a genotype associated with a large-seed phenotype, wherein the genetic locus comprises a GmKIX gene, and (b) denoting based on the genotyping that said soybean plant comprises a genotype associated a large-seed phenotype.

Also provided for herein is an edited soybean GmKIX gene comprising:

(i) a variant polynucleotide comprising a loss-of-function GmKIX gene variant, wherein the variant polynucleotide comprises at least one nucleotide insertion, deletion, and/or substitution in comparison to the corresponding unedited wild-type polynucleotide sequence, and wherein the variant polynucleotide exhibits reduced or loss of expression of the GmKIX gene in comparison to the wild-type polynucleotide sequence and/or encodes a GmKIX protein variant having reduced activity in comparison to wild-type GmKIX protein,

(ii) a variant polynucleotide encoding a loss-of-function GmKIX protein variant or fragment thereof, wherein the variant polynucleotide comprises at least one nucleotide insertion, deletion, and/or substitution in comparison to the corresponding unedited wild-type polynucleotide sequence and does not encode for the wild-type GmKIX8-1 protein, and wherein the variant polynucleotide encodes for a GmKIX8-1 protein variant having reduced activity in comparison to wild-type GmKIX8-1 protein;

(iii) a variant polynucleotide comprising a GmKIX gene 3′ UTR, wherein the variant polynucleotide comprises at least one nucleotide insertion, deletion, and/or substitution in the 3′ UTR in comparison to the corresponding unedited wild-type polynucleotide sequence, and wherein the variant 3′ UTR results in reduced or loss of expression of the GmKIX gene in comparison to the wild-type polynucleotide sequence;

(iv) a variant polynucleotide comprising a GmKIX gene 5′ UTR, wherein the variant polynucleotide comprises at least one nucleotide insertion, deletion, and/or substitution in the 5′ UTR in comparison to the corresponding unedited wild-type polynucleotide sequence, and wherein the variant 5′ UTR results in reduced or loss of expression of the GmKIX gene in comparison to the wild-type polynucleotide sequence;

(v) a variant polynucleotide comprising a GmKIX gene promoter, wherein the variant polynucleotide comprises at least one nucleotide insertion, deletion, and/or substitution in the promoter in comparison to the corresponding unedited wild-type polynucleotide sequence, and wherein the variant promoter results in reduced or loss of expression of the GmKIX gene in comparison to the wild-type polynucleotide sequence;

(vi) a variant polynucleotide comprising a GmKIX gene intron, wherein the variant polynucleotide comprises at least one nucleotide insertion, deletion, and/or substitution in the intron in comparison to the corresponding unedited wild-type polynucleotide sequence, and wherein the variant intron results in reduced or loss of expression of the GmKIX gene in comparison to the wild-type polynucleotide sequence; and/or

(vii) a variant polynucleotide comprising a GmKIX gene exon, wherein the variant polypeptide comprises at least one nucleotide insertion, deletion, and/or substitution in the exon in comparison to the corresponding unedited wild-type polynucleotide sequence, and wherein the variant exon results in reduced or loss of expression of the GmKIX gene in comparison to the wild-type polynucleotide sequence and/or encodes a GmKIX8-1 protein variant having reduced activity in comparison to wild-type GmKIX8-1 protein.

Also provided for are methods for producing a soybean plant comprising the edited soybean GmKIX, wherein edited soybean GmKIX gene exhibits reduced or loss of expression of the GmKIX8-1 gene in comparison to the wild-type polynucleotide sequence and/or encodes a GmKIX8-1 protein variant having reduced activity in comparison to wild-type GmKIX8-1 protein, the method comprising introducing into a plant cell one or more gene-editing molecules that target an endogenous soybean GmKIX8 gene to introduce at least one nucleotide insertion, deletion, and/or substitution into the endogenous GmKIX gene. In certain embodiments, the method comprises (i) providing to a plant cell, tissue, part, or whole plant an endonuclease or an endonuclease and at least one guide RNA, wherein the endonuclease or guide RNA and endonuclease can form a complex that can introduce a double-strand break at a target site in a genome of the plant cell, tissue, part, or whole plant; (ii) obtaining a plant cell, tissue, part, or whole plant wherein at least one nucleotide insertion, deletion, and/or substitution has been introduced into the corresponding wild-type polynucleotide sequence; and (iii) selecting a plant obtained from the plant cell, tissue, part or whole plant of step (ii) comprising the edited soybean GmKIX gene.

Also provided for herein are gene-edited soybean plants having a large-seed phenotype, wherein the soybean plant comprises a variant polynucleotide comprising a targeted loss-of-function GmKIX gene variant which comprises an insertion, substitution, and/or a deletion in a GmKIX gene that reduces expression of the GmKIX gene compared to wild-type expression and/or encodes a GmKIX protein variant having reduced activity in comparison to wild-type GmKIX protein.

Also provided for herein are methods of increasing soybean seed weight, the method comprising reducing or abolishing expression of the GmKIX gene and/or reducing or abolishing activity of the GmKIX9-1 protein.

### BRIEF DESCRIPTION OF THE DRAWINGS

FIG. 1A-I. FIG. 1 shows that soybean fast neutron (FN) mutant line K83 exhibited large leaf and seed phenotypes. (A) Representative photographs of fully expanded V4 leaves of wild-type (WT) and K83 mutant plants. (B) Leaf area measurements during early vegetative growth. C, cotyledon; U1, unifoliate leaf; V1-V4, first to fourth vegetative leaves. Mean values±SD for n=27 are shown. (C, D) Photographs (C) and 100-seed weight (D) of mature soybean seeds of Williams 82 and K83. Mean values±SD for n=27 are shown in (D). (E) Root fresh weight of Williams 82 and K83 at 30 d after germination. Mean values±SD for n=16 are shown. (F) Representative nail polish impression of abaxial cotyledon. (G-J) Quantification of cell number (G), stomatal index (H), and guard cell length (I), of abaxial epidermal cells of eight fully expanded cotyledons of Williams 82 and K83. Mean values±SD for n=8 are shown in (G-J). ***, P<0.001, according to Student's t-test. Bars: (A, C) 2 cm; (F) 50 μm.

FIG. 2A-E. FIG. 2 shows the identification of induced genetic deletions in K83 and co-segregation of large leaf and seed size phenotypes with chromosome 17 deletion. (A) Comparative genome hybridization (CGH) between Williams 82 and K83 showing deleted regions in chromosomes 6, 17, 18 and 19. Arrows indicate the predicted deletions that were used for molecular mapping. The y-axis represents normalized log 2 ratios of K83 to Williams 82 hybridization signals. (B) Actual coordinates of the deletions are shown. (C) Large leaf phenotype co-segregated with homozygous deletion in chromosome 17 in BC1F3 K83 plants. Four plants derived from BC1F2 families showing large (F3-3 and F3-10) and wild-type leaves were genotyped by polymerase chain reaction (PCR) using specific primers for each of the deletions in chromosomes 6, 17, 18 and 19. Control PCR reactions were performed using specific primers for Glyma.02g012600 encoding a soybean legume lectin domain protein. (D) Quantitative real-time PCR analysis of the expression of GmKIX8-1 and GmKIX8-2 in leaves of Williams 82 and gmkix8-1 plants. Mean values±SD for n=4 are shown. P<0.05, according to Student's t-test, nd: not detectable. (E) Copy number of GmKIX8-1 in heterozygous BC1F3 (F3-11) K83 plants using genomic quantitative PCR (qPCR) (top panel) and associated leaf and seed phenotypes (lower panel). B, Big; N, normal. Mean values±SD are shown.

FIG. 3A-G. FIG. 3 shows soybean plants harboring a CRISPR/Cas9-induced GmKIX8-1 deletion produced large leaves and seeds. (A) Diagram of dual gRNA CRISPR/Cas9 vector (upper panel), sgRNA sequences (middle panel) and locations of target 1 and target 2 in the first and second exons of GmKIX8-1, respectively (lower panel). Gene-specific primers used for polymerase chain reaction (PCR) genotyping (arrows) and expected PCR amplicon size are indicated in the lower panel. BAR, Basta selection gene; hCAS9, human Cas9; gRNA, guide RNA; LB, T-DNA left border; MAS, Mannopine Synthase; MAS-Ter, MAS terminator; NLS, nuclear localization signal; Nos-Ter, Nopaline Synthase terminator; pU6, Arabidopsis U6 promoter; RB, T-DNA right border; 3×FLAG, 3× FLAG sequences; 2x35S, double-enhancer CAMV 35S promoter. (B) PCR-based genotyping of GmKIX8-1 (upper panel) and GmKIX8-2 (lower panel) in T0, T1 and Maverick (WT) plants. Higher mobility amplicons indicate CRISPR/Cas9-induced deletion in GmKIX8-1. (C) Sanger sequencing of GmKIX8-1 wild-type (upper PCR band in (B)) and mutant (lower PCR band in (B)) amplicons. Wild-type amplicons showed wild-type sequences (CRISPR-WT) while mutant amplicons showed a 214 bp deletion due to excision of DNA sequences upstream of the PAM site (CRISPR-KIX). (D, E) Representative photographs of leaves (D) and seeds (E) of Maverick, CRISPR-WT and CRISPR-KIX plants. (F, G) Unifoliate leaf area (F) and 100-seed weight (G) of Maverick, CRISPR-WT and homozygous CRISPR-KIX plants. Mean values±SD for n=12 (F), and n=27 (G) are shown. Statistical analysis was done by one-way ANOVA followed by a post-hoc Tukey's multiple range test. Different letters denoted significant differences at P<0.001. Bars: (D, E) 2 cm. gRNA1 with PAM 5′-GGCCTTACGAGTGCGTGAGAAGG (SEQ ID NO: 72); complement gRNA1 with PAM 3′-CCGGAATGCTCACGCACTCTTCC (SEQ ID NO: 73); gRNA2 with PAM 5′-GCTCCCCGTGGTGGTTCTCAAGG (SEQ ID NO: 74); complement gRNA2 with PAM 3′-CGAGGGGCACCACCAAGAGTTCC (SEQ ID NO: 75).

FIG. 4A-F. FIG. 4 shows the subcellular localization and downstream targets of GmKIX8-1. (A) Subcellular localization of GFP (green fluorescent protein; upper panels) and GmKIX8-1-GFP (lower panels) fusion in tobacco leaf epidermal cells. Proteins were expressed from the constitutive CAMV 35S promoter. Nuclei were visualized by 40,6-diamidino-2-phenylindole (DAPI). (B) Western-blot analysis showing GFP and GmKIX8-1-GFP expression in infiltrated tobacco leaves. (C-F) Transcript expression of CYCD3;1-10 (C), CYCD3;2-17 and CYCD3;3-05 (D), GRF1-10 and GFR1-20 (E), and PPD10 and PPD20 (F). Gene expression was determined in shoot tips by quantitative real-time polymerase chain reaction (qRT-PCR). Mean values±SD for n=4 are shown. **, P<0.01 according to Student's t-test.

FIG. 5A-E. FIG. 5 shows the mapped locations of QTL qSw17-1 for the big-seeded phenotype in soybean overlap GmKIX8-1. (A) Diagram of mapped qSw17-1 locations in chromosome 17. The location of the K83 deletion and encoded genes within the deletion (arrows) are also shown. GmKIX8-1 is indicated by the circled arrow. (B, C) Representative photographs (B) and 100-seed weights (C) of mature soybean seeds from Williams 82, Maverick and the big-seeded PI597483. Bar, 2 cm. Mean values±SD for n=20 are shown in (C). (D, E) Representative nail polish impression images (D) and quantification of cell number (E) in the abaxial epidermal layer of eight fully expanded cotyledons of Williams 82, Maverick and PI597483. Bar, 50 μm. Mean values±SD for n=8 are shown in (E). Statistical analysis was performed using one-way ANOVA followed by a post-hoc Tukey's multiple range test. Different letters denote significant differences at P<0.05 in (C) and (E).

FIG. 6A-E. FIG. 6 shows that GmKIX8-1 expression is downregulated in big-seeded plant introductions (PIs) harboring QTL qSw17-1. (A) Diagram showing polymorphisms in the GmKIX8-1 promoter and coding sequences of qSw17-1 compared to Williams 82 (W82) sequences. The big-seeded PIs harbor two deletions upstream of the start codon, two nucleotide additions in the 3′UTR and eight nucleotide substitutions in the coding region of GmKIX8-1. GACACGCCGCCAC (SEQ ID NO: 76). GTTTTGGTGTGTGTGTGTGTGTG (SEQ ID NO: 77). (B, C) Quantitative reverse transcription polymerase chain reaction (qRT-PCR) analyses of GmKIX8-1 (B), and GmCYD3;1-10 (C), in shoot tips of Maverick, Williams 82 and PI597483. Mean values±SD for n=4 are shown. (D) Schematic representation of GmKIX8-1-promoter luciferase (Luc) reporter constructs. Black boxes indicate CGC repeated sequence, grey boxes indicate GT rich repeated sequence, ‘X’ indicates mutated region. Promoter sequences are: Williams 82 (SEQ ID NO: 67); PI597483 (SEQ ID NO: 68); Del 1 (SEQ ID NO: 69); Del 2 (SEQ ID NO: 70); Del 3 (SEQ ID NO: 71). (E) Relative LUC activity (firefly LUC/renilla LUC) of promoter-LUC constructs depicted in diagram form in (D) in N. benthamina transient expression assays. Mean values±SD for n=4 are shown. Statistical analysis was performed using one-way ANOVA followed by a post-hoc Tukey's multiple range test. Different letters denote significant differences at P<0.05 in (B), (C) and (E).

FIG. 7A,B. FIG. 7 shows detached banner, wing, and keel petals of the soybean wild-type (A) and fast neutron mutant K83 line (B). Scale bar 2 mm.

FIG. 8. FIG. 8 shows a phylogenetic tree of GmKIX8 orthologues in various plant species.

FIG. 9A-C. FIG. 9 shows Sequencing of GmKIX8-2 (A) and expression analysis of GmKIX8-1 (B) and GmKIX8-2 (C) in Maverick and CRISPR-mutants. Shown are means±SD for n=6, One-way ANOVA with post hoc Tukey HSD test. Different letters denoted significant differences p<0.01.

FIG. 10A-I. FIG. 10 shows the phenotypes associated with CRISPR-induced mutation in GmKIX8-1. Fully expanded of unifoliate (A), V4 (B) leaves and flowers (C) of the WT and CRISPR mutant. (D) Nail polish impression image and quantification of cell number (E), stomatal index (F) and guard cell length (G) of abaxial epidermal cell of eight fully expanded cotyledons in WT and CRISPR-KIX using ImageJ software, shown are means±SD for n=8. ***, P<0.001, student t-test. Seed weight (H) and seed number (I) measurement of mature soybean seeds from CRISPR plants (CRISPR-WT, no mutation, CRISPR-Het, heterozygous mutation and CRISRP-KIX Homozygous mutant gmkix8-1 growth in the greenhouse. Shown are mean±SD, for n=13, one-way ANOVA followed by a post-hoc Turkey's multiple range test. Different letters denoted significant differences at p<0.01. Scale bar, 2 cm (A, B); 2 mm (C); 50 μm (D).

FIG. 11A,B. FIG. 11 shows the expression profile of GmKIX8-1 and GmKIX8-2 in different soybean Williams 82 tissues. (A) mRNA expression determined by qRT-PCR. Shown are means±SD for n=4, One-way ANOVA with post hoc Tukey HSD test, different letters indicate significant differences between tissues p<0.01. (B) Transcript expression in publicly available RNA-seq database (on the world wide web at phytozome.jgi.doe.gov/).

FIG. 12A-C. FIG. 12 shows seed and leaf phenotypes of different cultivated soybean cultivars. (A) Photographs of seeds. (B) 100-Seed Weight. Shown are means±SD for n=6, One-way ANOVA with post hoc Tukey HSD test, p<0.001. Scale bar=1 cm. (C) Photographs of V3 leaves four weeks after germination under greenhouse condition. Scale bar=5 cm. PI597483 and PI561369 are big-seeded cultivars encoding QTL qSW17-1.

FIG. 13. FIG. 13 shows simple sequence repeat (SSR) marker developed for identifying the big seeded phenotype in PI594021, PI597483 and PI561396 harboring QTL qSW17-1.

FIG. 14. FIG. 14 shows protein alignment of GmKIX8-1 from different soybean cultivars. The KIX domain, B domain, and the EAR motif are indicated. Polymorphic amino acids are in colored fonts. PI594021, PI597483 and PI561369 are big-seeded cultivars encoding QTL qSW17-1.

FIG. 15. FIG. 15 shows the sub-cell localization of GmKIX8-1 derived from PI597483 fused to green fluorescent protein (GFP) in tobacco cells

FIG. 16. FIG. 16 shows the identification of Cis-element containing GT repeat sequences in the promoter region of GmKIX8-1.

FIG. 17. FIG. 17 shows an alignment of 4.6 kb GmKIX8-1 DNA sequences from Williams 82 (SEQ ID NO: 78) and plant introductions PI597483 (PI83) (SEQ ID NO: 79), PI594021 (PI21) (SEQ ID NO: 80), and PI561396 (PI96) (SEQ ID NO: 81). Start and stop codons are in red, deletions are shaded in red, additions are shaded in purple, and substitutions are shaded in green.

FIG. 18. FIG. 18 shows an alignment of ˜1.6 kb DNA Sequences for Williams 82, PI597483, and the three promoter deletion versions (del1, del2, and del3) are shown. Deletions are shaded.

### DETAILED DESCRIPTION

### Definitions

The headings provided herein are solely for ease of reference and are not limitations of the various aspects or aspects of this disclosure, which can be had by reference to the specification as a whole.

The practice of the present invention will employ, unless otherwise indicated, conventional techniques of botany, microbiology, tissue culture, molecular biology, chemistry, biochemistry, recombinant DNA technology, and bioinformatics which are within the ordinary skill of the art.

Unless defined otherwise, technical and scientific terms used herein have the same meaning as commonly understood by one of ordinary skill in the art to which this disclosure is related.

The term “and/or” where used herein is to be taken as specific disclosure of each of the two specified features or components with or without the other. Thus, the term and/or” as used in a phrase such as “A and/or B” herein is intended to include “A and B,” “A or B,” “A” (alone), and “B” (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

The term “comprising” as used herein is to be construed as at least having the features to which it refers while not excluding any additional unspecified features. However, in embodiments provided herein where the term “comprising” is used, other embodiments where the phrases “consisting of” and/or “consisting essentially of” are substituted for the term “comprising” are also provided.

As used herein, the terms “include,” “includes,” and “including” are to be construed as at least having the features to which they refer while not excluding any additional unspecified features.

Where a term is provided in the singular, other embodiments described by the plural of that term are also provided. For example, the term “a” or “an” entity refers to one or more of that entity; “an allele,” is understood to represent “one or more alleles.” As such, the terms “a” (or “an”), “one or more,” and “at least one” can be used interchangeably herein.

Numeric ranges are inclusive of the numbers defining the range. Even when not explicitly identified by “and any range in between,” or the like, where a list of values is recited, e.g., 1, 2, 3, or 4, the disclosure specifically includes any range in between the values, e.g., 1 to 3, 1 to 4, 2 to 4, etc.

As used herein, an “allele” refers to one of two or more alternative forms of a genomic sequence at a given locus on a chromosome. When all the alleles present at a given locus on a chromosome are the same, that plant is homozygous at that locus. If the alleles present at a given locus on a chromosome differ, that plant is heterozygous at that locus.

As used herein, the term “denoting” when used in reference to a plant genotype refers to any method whereby a plant is indicated to have a certain genotype. Such indications of a certain genotype include, but are not limited to, any method where a plant is physically marked or tagged. Physical markings or tags that can be used include, but not limited to, a barcode, a radio-frequency identification (RFID) tag, a label, or the like. Indications of a certain genotype also include, but are not limited to, any entry into any type of written or electronic database whereby the plant's genotype is provided.

A “genetic locus,” “genomic locus,” or just “locus” is a position on a genomic sequence that is usually found by a point of reference; e.g., a short DNA sequence that is a gene, or part of a gene or intergenic region. A locus may refer to a nucleotide position at a reference point on a chromosome, such as a position from the end of the chromosome. While the genetic locus may be identified by a particular reference sequence, e.g., the soybean GmKIX8-1 gene Glyma.17G112800I (SEQ ID NO: 78), it is understood that the locus can comprise various allelic forms or variants and/or be from various soybean cultivars and still be considered the same locus or genetic marker.

As used herein, “linkage” refers to relative frequency at which types of gametes are produced in a cross. For example, if locus A has genes “A” or “a” and locus B has genes “B” or “b” and a cross between parent I with AABB and parent B with aabb will produce four possible gametes where the genes are segregated into AB, Ab, aB and ab. The null expectation is that there will be independent equal segregation into each of the four possible genotypes, i.e. with no linkage ¼ of the gametes will of each genotype. Segregation of gametes into a genotypes differing from ¼ are attributed to linkage.

As used herein, the termed “linked”, when used in the context of markers and/or genomic regions, means that the markers and/or genomic regions are located on the same linkage group or chromosome.

As used herein, “polymorphism” means the presence of one or more variations of a nucleic acid sequence at one or more loci in a population of at least two members. The variation can comprise but is not limited to one or more nucleotide base substitutions, the insertion of one or more nucleotides, a nucleotide sequence inversion, and/or the deletion of one or more nucleotides.

As used herein, the term “single nucleotide polymorphism,” also referred to by the abbreviation “SNP,” means a polymorphism at a single site wherein the polymorphism constitutes any or all of a single base pair change, an insertion of one or more base pairs, and/or a deletion of one or more base pairs.

As used herein, “marker” means a detectable characteristic that can be used to discriminate between organisms. Examples of such characteristics include, but are not limited to, genetic markers, biochemical markers, morphological characteristics, and agronomic characteristics.

As used herein, “marker assay” means a method for detecting a polymorphism at a particular locus using a particular method. Marker assays thus include, but are not limited to, measurement of at least one phenotype (such as seed color, flower color, plant height, seed size, seed weight, disease or herbicide resistance, or other visually detectable trait as well as any biochemical trait), or genotyping such as by restriction fragment length polymorphism (RFLP), single base extension, electrophoresis, sequence alignment, allelic specific oligonucleotide hybridization (ASO), random amplified polymorphic DNA (RAPD), microarray-based polymorphism detection technologies, and the like.

As used herein, “genotype” means the genetic component of the phenotype that can be indirectly characterized using markers or directly characterized by nucleic acid sequencing.

As used herein, “phenotype” means the detectable characteristics of a cell or organism which can be influenced by gene expression.

As used herein, the term “introgressed”, when used in reference to a genetic locus, refers to a genetic locus that has been introduced into a new genetic background. Introgression of a genetic locus can thus be achieved through both plant breeding methods or by molecular genetic methods. Such molecular genetic methods include, but are not limited to, various plant transformation techniques and/or methods that provide for homologous recombination, non-homologous recombination, site-specific recombination, and/or genomic modifications that provide for locus substitution or locus conversion. In certain embodiments, introgression could thus be achieved by substitution of a genetic locus comprising a non-large seed size genotype with a corresponding genetic locus comprising a large seed size genotype, such as through crossing, or by conversion of a genetic locus comprising a non-large seed size genotype to a large-seed size genotype, such as by gene editing.

As used herein, “quantitative trait locus (QTL)” means a locus that controls to some degree numerically representable traits that are usually continuously distributed.

As used herein, the term “soybean” comprises Glycine max and all plant varieties that can be bred with Glycine max. Certain embodiments consist of Glycine max.

As used herein, the term “bulk” refers to a method of managing a segregating population during inbreeding that involves growing the population in a bulk plot, harvesting the self-pollinated seed of plants in bulk, and using a sample of the bulk to plant the next generation.

As used herein, a polynucleotide is said to be “endogenous” to a given cell when it is found in a naturally occurring form and genomic location in the cell.

As used herein, the phrase “consensus sequence” refers to an amino acid, DNA or RNA sequence created by aligning two or more homologous sequences and deriving a new sequence having either the conserved or set of alternative amino acid, deoxyribonucleic acid, or ribonucleic acid residues of the homologous sequences at each position in the created sequence.

As used herein, the terms (gene . . . , genome . . . , genetic . . . , and the like) “edit,” “editing,” “edited,” and the like refer to processes or products where insertions, deletions, and/or nucleotide substitutions are introduced into a genome. Such processes include methods of inducing homology directed repair and/or non-homologous end joining of one or more sites in the genome.

The phrases “genetically edited plant,” “edited plant,” and the like are used herein to refer to a plant or progeny thereof comprising one or more human-introduced nucleotide insertions, deletions, substitutions, or any combination thereof in the genomic DNA of the plant. Such genetically edited plants can be constructed by techniques including CRISPR/Cas endonuclease-mediated editing, meganuclease-mediated editing, engineered zinc finger endonuclease-mediated editing, and the like.

The term “heterologous,” as used herein in the context of a second polynucleotide that is operably linked to a first polynucleotide, refers to: (i) a second polynucleotide that is derived from a source distinct from the source of the first polynucleotide; (ii) a second polynucleotide derived the same source as the first polynucleotide, where the first, second, or both polynucleotide sequence(s) is/are modified from its/their original form; (iii) a second polynucleotide arranged in an order and/or orientation or in a genomic position or environment with respect to the first polynucleotide that is different than the order and/or orientation in or genomic position or environment of the first and second polynucleotides in a naturally occurring cell; or (iv) the second polynucleotide does not occur in a naturally occurring cell that contains the first polynucleotide. Heterologous polynucleotides include polynucleotides that promote transcription (e.g., promoters and enhancer elements), transcript abundance (e.g., introns, 5′ UTR, and 3′ UTR), translation, or a combination thereof as well as polynucleotides encoding peptides or proteins, spacer peptides, or localization peptides. In certain embodiments, a nuclear or plastid genome can comprise the first polynucleotide, where the second polynucleotide is heterologous to the nuclear or plastid genome. A “heterologous” polynucleotide that promotes transcription, transcript abundance, translation, or a combination thereof as well as polynucleotides encoding peptides, spacer peptides, or localization peptides can be autologous to the cell but, however, arranged in an order and/or orientation or in a genomic position or environment that is different than the order and/or orientation in or genomic position or environment in a naturally occurring cell. A polynucleotide that promotes transcription, transcript abundance, translation, or a combination thereof as well as polynucleotides encoding peptides, spacer peptides, or localization can be heterologous to another polynucleotide when the polynucleotides are not operably linked to one another in a naturally occurring cell. Heterologous peptides or proteins include peptides or proteins that are not found in a cell or organism as the cell or organism occurs in nature. As such, heterologous peptides or proteins include peptides or proteins that are localized in a subcellular location, extracellular location, or expressed in a tissue that is distinct from the subcellular location, extracellular location, or tissue where the peptide or protein is found in a cell or organism as it occurs in nature. Heterologous polynucleotides include polynucleotides that are not found in a cell or organism as the cell or organism occurs in nature.

The term “homolog” as used herein refers to a gene related to a second gene by identity of either the DNA sequences or the encoded protein sequences. Genes that are homologs can be genes separated by the event of speciation (see “ortholog”). Genes that are homologs can also be genes separated by the event of genetic duplication (see “paralog”). Homologs can be from the same or a different organism and can in certain embodiments perform the same biological function in either the same or a different organism.

The phrase “operably linked” as used herein refers to the joining of nucleic acid or amino acid sequences such that one sequence can provide a function to a linked sequence. In the context of a promoter, “operably linked” means that the promoter is connected to a sequence of interest such that the transcription of that sequence of interest is controlled and regulated by that promoter. When the sequence of interest encodes a protein that is to be expressed, “operably linked” means that the promoter is linked to the sequence in such a way that the resulting transcript will be efficiently translated. If the linkage of the promoter to the coding sequence is a transcriptional fusion that is to be expressed, the linkage is made so that the first translational initiation codon in the resulting transcript is the initiation codon of the coding sequence. Alternatively, if the linkage of the promoter to the coding sequence is a translational fusion and the encoded protein is to be expressed, the linkage is made so that the first translational initiation codon contained in the 5′untranslated sequence associated with the promoter and the coding sequence is linked such that the resulting translation product is in frame with the translational open reading frame that encodes the protein. Nucleic acid sequences that can be operably linked include sequences that provide gene expression functions (e.g., gene expression elements such as promoters, 5′ untranslated regions, introns, protein coding regions, 3′ untranslated regions, polyadenylation sites, and/or transcriptional terminators), sequences that provide DNA transfer and/or integration functions (e.g., T-DNA border sequences, site specific recombinase recognition sites, integrase recognition sites), sequences that provide for selective functions (e.g., antibiotic resistance markers, biosynthetic genes), sequences that provide scoreable marker functions (e.g., reporter genes), sequences that facilitate in vitro or in vivo manipulations of the sequences (e.g., polylinker sequences, site specific recombination sequences) and sequences that provide replication functions (e.g., bacterial origins of replication, autonomous replication sequences, centromeric sequences). In the context of an amino acid sequence encoding a localization, spacer, linker, or other peptide, “operably linked” means that the peptide is connected to the polyprotein sequence(s) of interest such that it provides a function. Functions of a localization peptide include localization of a protein or peptide of interest to, e.g., an extracellular space or subcellular compartment. Functions of a spacer peptide include linkage of two peptides of interest such that the peptides will be expressed as a single protein.

As used herein, the term “polypeptide” is intended to encompass a singular “polypeptide” as well as plural “polypeptides,” and comprises any chain or chains of two or more amino acids. Thus, as used herein, a “peptide,” an “oligopeptide,” a “dipeptide,” a “tripeptide,” a “protein,” an “amino acid chain,” an “amino acid sequence,” “a peptide subunit,” or any other term used to refer to a chain or chains of two or more amino acids, are included in the definition of a “polypeptide,” (even though each of these terms can have a more specific meaning) and the term “polypeptide” can be used instead of, or interchangeably with any of these terms. The term further includes polypeptides which have undergone post-translational modifications, for example, glycosylation, acetylation, phosphorylation, amidation, derivatization by known protecting/blocking groups, proteolytic cleavage, or modification by non-naturally occurring amino acids.

The phrases “percent identity” or “sequence identity” as used herein refer to the number of elements (i.e., amino acids or nucleotides) in a sequence that are identical within a defined length of two DNA, RNA or protein segments (e.g., across the entire length of a reference sequence) in an alignment resulting in the maximal number of identical elements, and is calculated by dividing the number of identical elements by the total number of elements in the defined length of the aligned segments and multiplying by 100.

The phrase “transgenic plant” refers to a plant or progeny thereof wherein the plant's or progeny plant's DNA of the nuclear or plastid genome contains an introduced exogenous DNA molecule of 10 or more nucleotides in length. Such introduced exogenous DNA molecules can be naturally occurring, non-naturally occurring (e.g., synthetic and/or chimeric), from a heterologous source, or from an autologous source.

As used herein, a “control” plant is a plant (or a member of a population of plants) recognized as having a representative phenotype (e.g., leaf size, seed size, seed number, height, flower number, etc.) of a soybean plant that does not comprise the genotype associated with large-seed phenotype of this disclosure, but is otherwise similar in genetic makeup. For example, one of ordinary skill in the art would understand a control plant to have one or more of the following attributes: has at least one parent in common with the treated plant; shares a common ancestor with the treated plant within twelve generations; shares sufficient common genetic heritage with the treated plant that one of ordinary skill in the art of plant breeding would recognize the control plant as a valid comparison for establishing a correlation between a genotype and the resulting phenotype; and/or achieves a morphology considered typical of the wild-type plant. One of ordinary skill in the art will recognize that a control plant that by chance (e.g., a statistical outlier), by some other type of manipulation, or other reason comprises a phenotype that varies from a representative phenotype of control plants would not be an appropriate control plant for comparison.

As used herein, “reduced expression” means values that are statistically lower (P<0.05) compared to expression of the unaltered and/or wild-type version of a gene. In certain embodiments, reduced expression includes a complete or near complete loss of expression (abolish). In certain embodiments, reduced expression can be at least 2%, 3%, 4%, 5%, 6%, 7%, 8%, 9%, 10%, 15%, 20%, 25%, 30%, 40%, 50%, 75%, or 95% lower compared to expression of the unaltered and/or wild-type version of a gene, or any range or value lying therein.

As used herein, “reduced activity” means values that are statistically lower (P<0.05) compared to activity of the unaltered and/or wild-type version of a protein. In certain embodiments, reduced expression includes a complete or near complete loss of activity (abolish). In certain embodiments, reduced activity can be at least 2%, 3%, 4%, 5%, 6%, 7%, 8%, 9%, 10%, 15%, 20%, 25%, 30%, 40%, 50%, 75%, or 95% lower compared to activity of the unaltered and/or wild-type version of a protein, or any range or value lying therein.

As used herein, “large-seed phenotype,” “exhibits a large-seed phenotype,” and the like, refers to a phenotypic trait observed in certain soybean plants in which the seeds produced by the soybean plant have significantly higher seed weight (P<0.05) compared to “Wild-type.” Unless otherwise specified, Wild-type soybean seed weight is the seed weight of commercial Glycine max cultivars. In certain embodiments, Wild-type seed weight refers specifically to the seed weight of the corresponding parental unmodified soybean cultivar. In certain embodiments, a large-seed phenotype means having a seed weight that is 2%, 3%, 4%, 5%, 6%, 7%, 8%, 9%, 10%, 15%, 20%, 25%, 30%, 40%, or 50% higher compared to a control and/or Wild-type seed weight, or any rang or value lying therein.

To the extent to which any of the preceding definitions is inconsistent with definitions provided in any patent or non-patent reference incorporated herein by reference, any patent or non-patent reference cited herein, or in any patent or non-patent reference found elsewhere, it is understood that the preceding definition will be used herein.

### Overview

Mutant populations are valuable genetic resources for identifying genetic variations and studying gene function in plants. In soybean, several mutant populations have been created using various mutagenic agents such as chemical, irradiation, or transposon (Campbell & Stupar, 2016). More recently, fast neutron (FN) mutant populations were utilized to identify and characterize several causative genes or genetic loci for important seed composition phenotypes in soybean such as increased production of vitamin E (Stacey et al., 2016), sucrose (Dobbels et al., 2017), stearic acid (Gillman et al., 2014), reduced seed phytic acid (Vin-cent et al., 2015) or altered plant morphology (Bolon et al., 2011; Hwang et al., 2015). One advantage of FN mutagenesis is the induction of genetic deletions which can be rapidly identified by comparative genome hybridization (CGH). Moreover, given the relative narrow genetic diversity in domesticated soybean populations (Hyten et al., 2006), FN mutagenesis can generate diverse mutations that can allow the identification of novel genes involved in important agronomic traits in soybean. In this study, we identified a soybean AtKIX8 ortholog, GmKIX8-1, involved in the control of cell proliferation and organ size through forward and reverse genetics using FN mutagenesis and CRISPR/Cas9 genome editing, respectively. Our data also indicate that reduction in GmKIX8-1 expression is the genetic basis for the big seed phenotype of soybean plant introductions (PIs) harboring the major seed weight QTL qSw17-1. Based on the nucleotide polymorphisms in the GmKIX8-1 promoter encoded in qSw17-1, we developed a simple sequence repeat (SSR) marker that may be useful for marker-assisted selection for seed size in soybean.

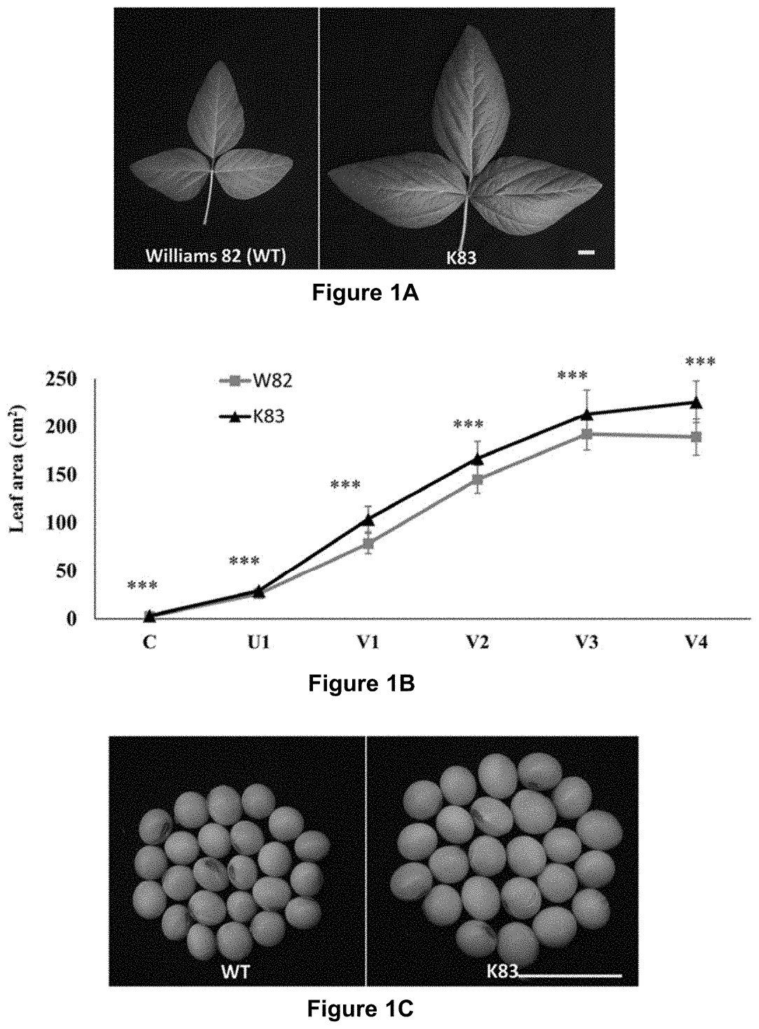

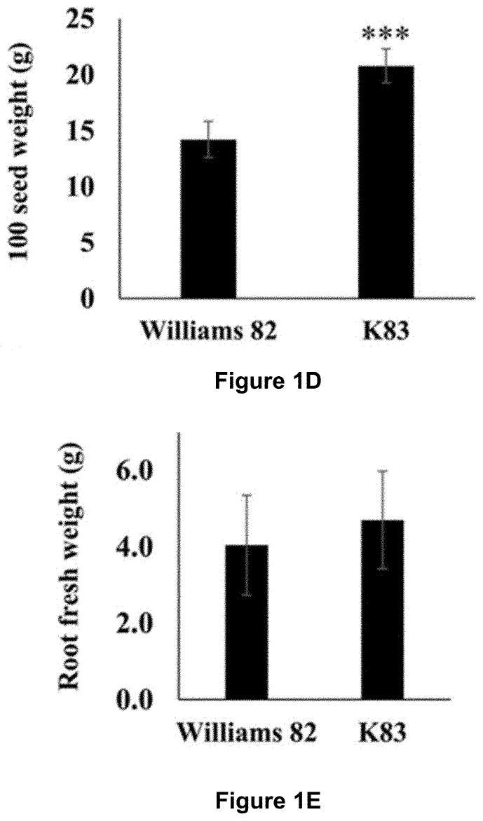

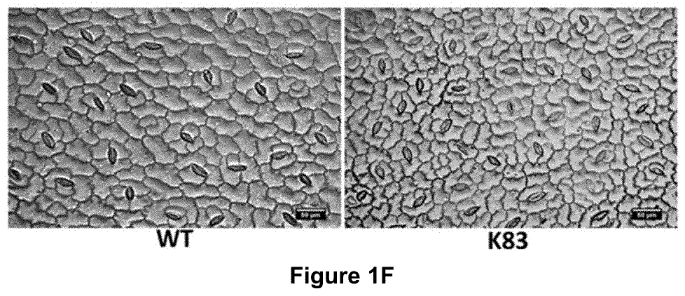

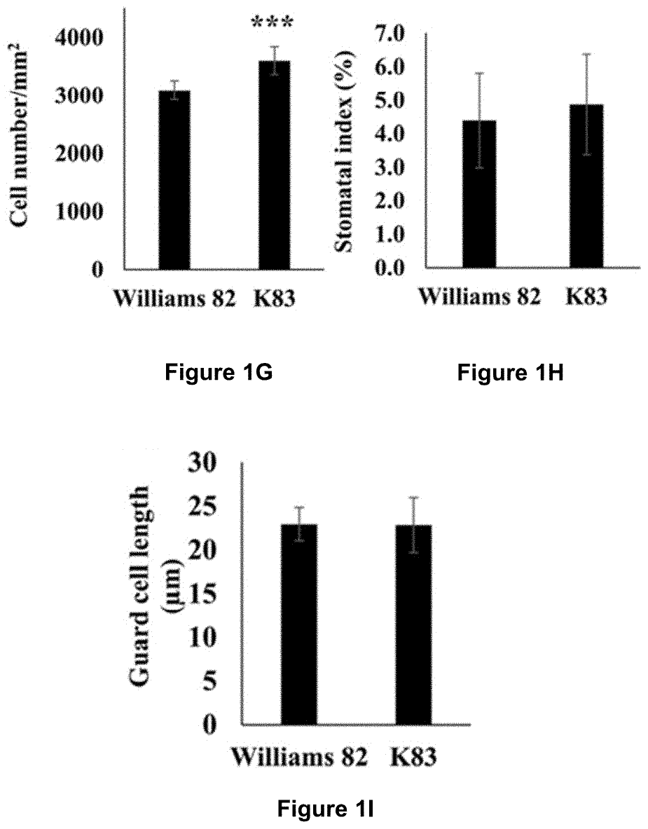

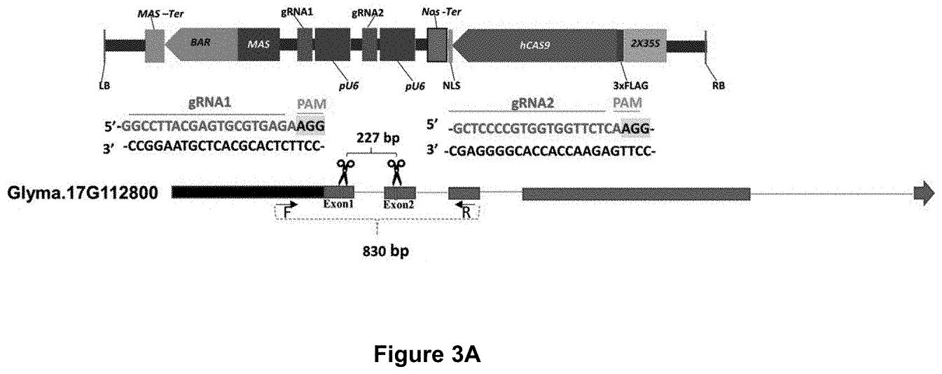

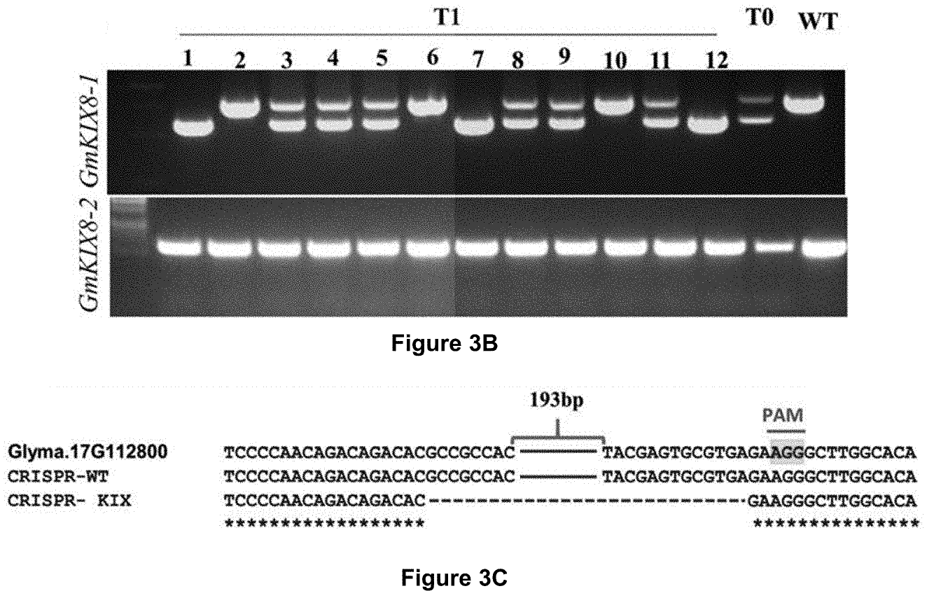

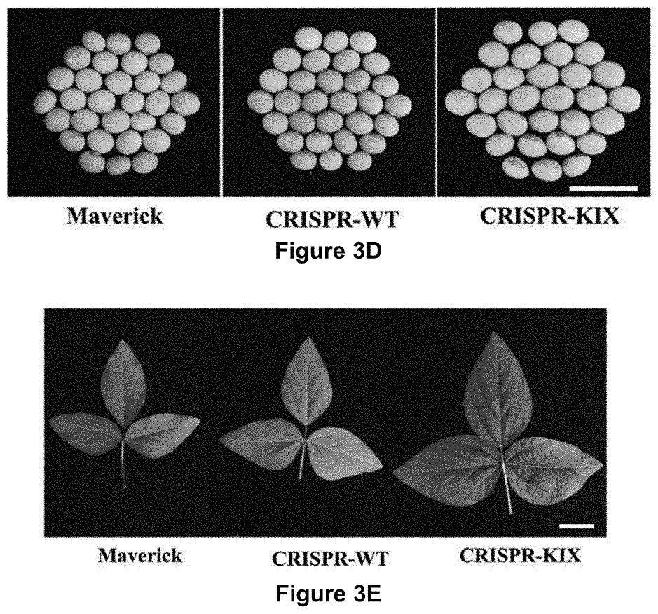

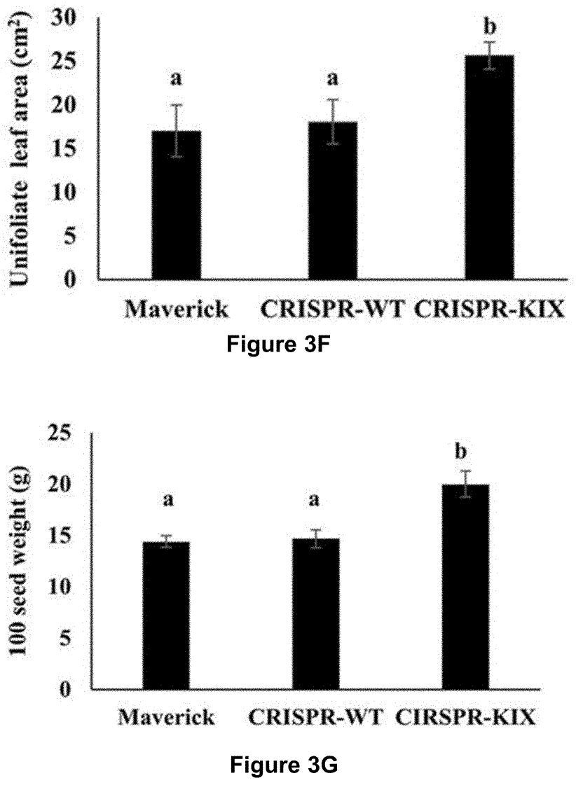

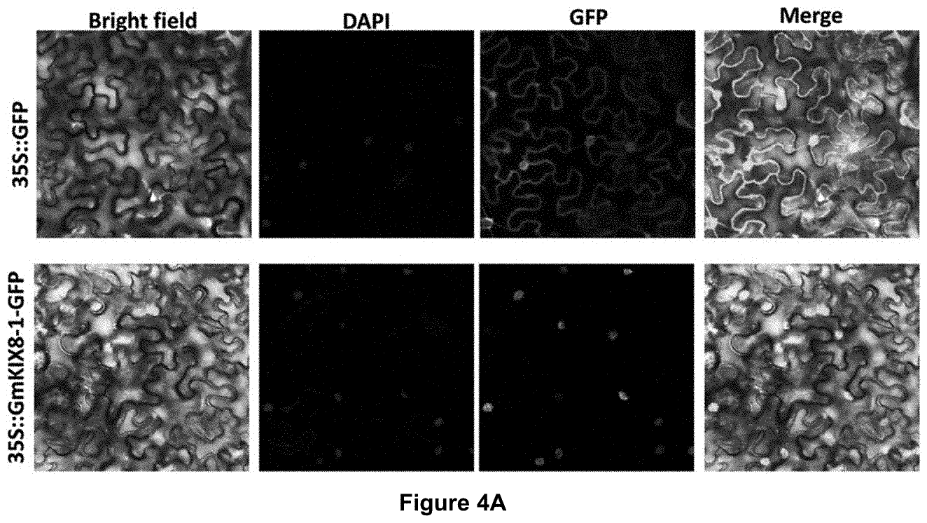

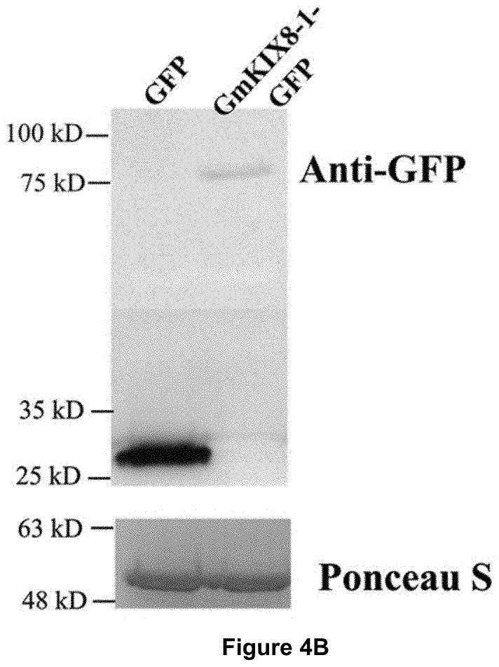

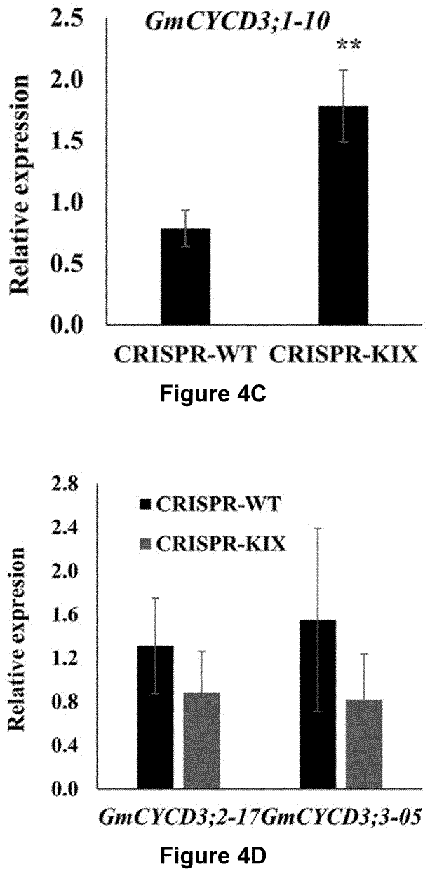

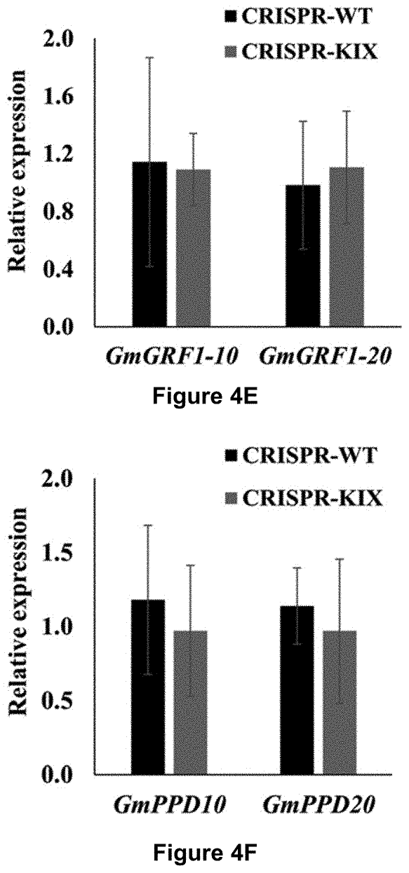

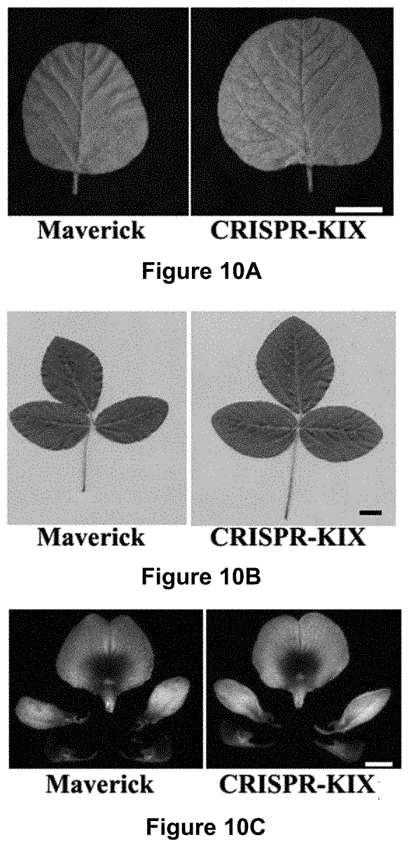

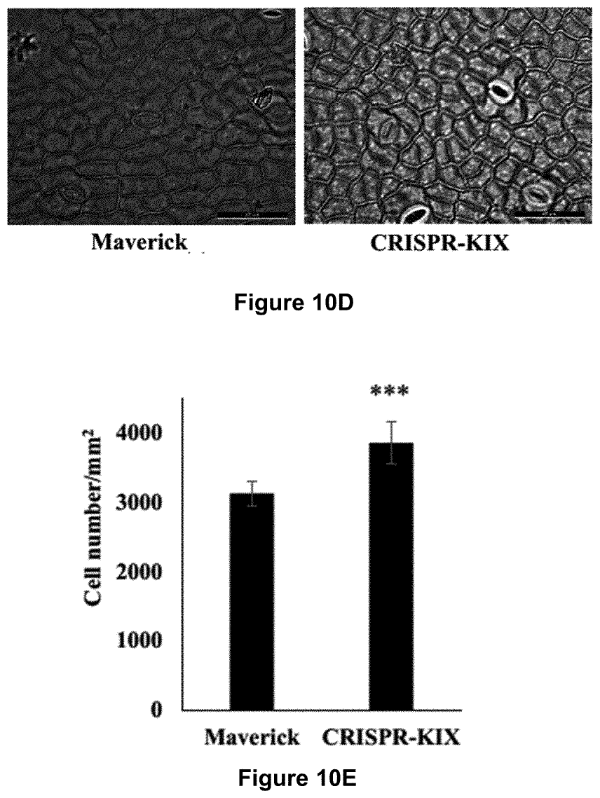

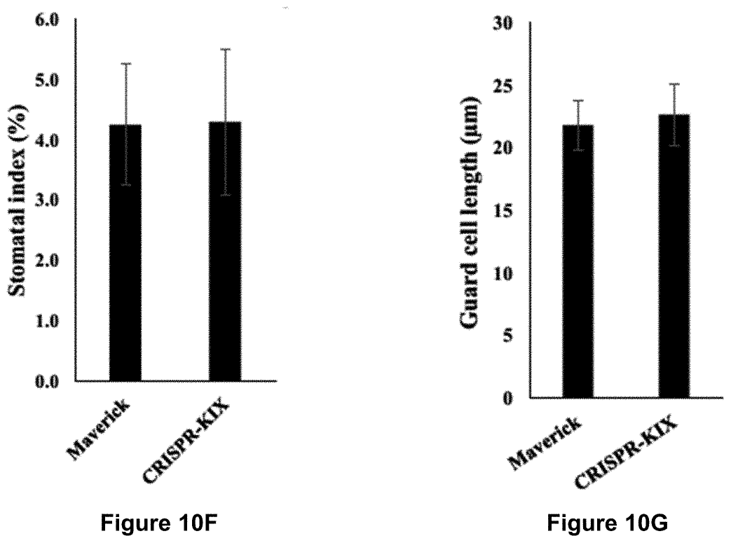

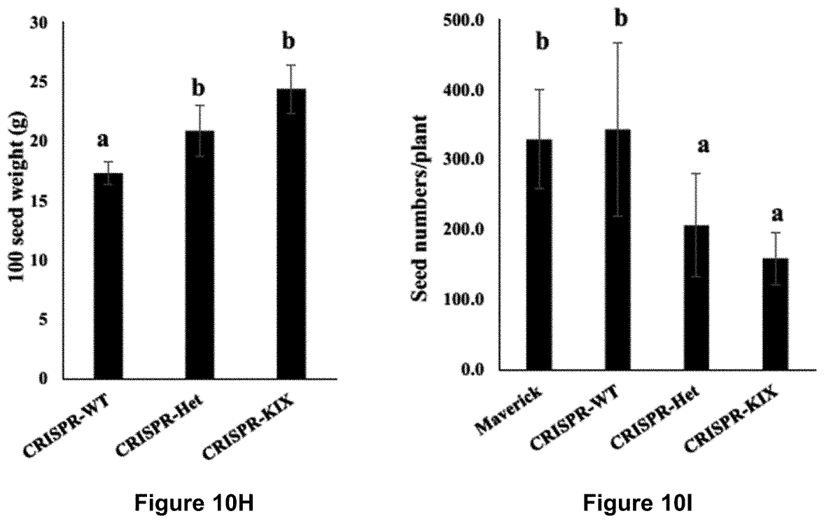

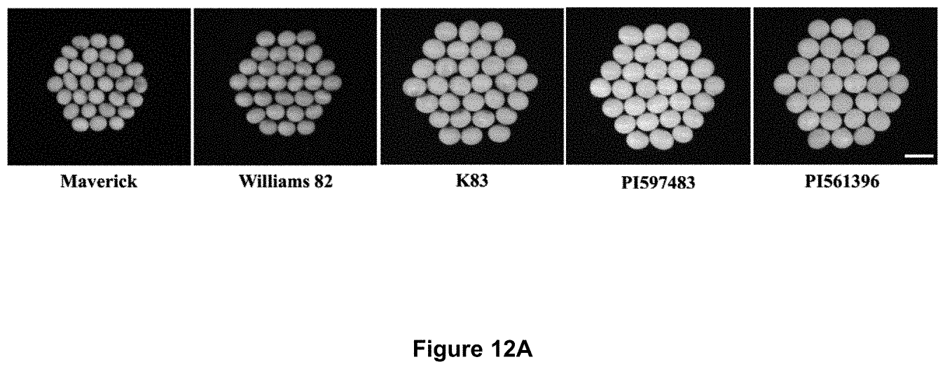

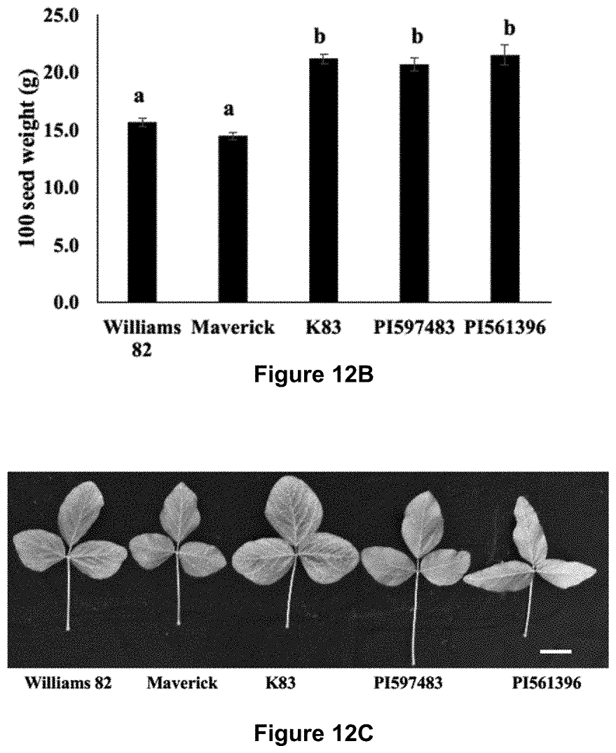

Disclosed herein and characterized is GmKIX8-1 (Glyma.17G112800), a gene encoding a nuclear protein that regulates organ size in soybean. GmKIX8-1 encodes a conserved KIX domain protein and is orthologous to AtKIX8 in Arabidopsis, which restricts organ growth by regulating meristemoid cell proliferation (White, 2006; Gonzalez et al., 2015). Loss of function of AtKIX8 and AtKIX9 in Arabidopsis lead to prolonged division of meristemoids and consequently to increased numbers of epidermal cells that surround stomata and to a reduced stomatal index (White, 2006; Gonzalez et al., 2015; Li et al., 2018a). AtKIX8 and AtKIX9 interact with AtPEAPOD (AtPPD) proteins and act as adaptors to recruit the transcription repressor AtTOPLESS (AtTPL). In agreement with the function of the PPD/KIX/TPL repressor complex in controlling organ size, the soybean gmkix8-1 null mutants produced bigger seeds and leaves (FIG. 1A-D; FIG. 3D-G; FIG. 10H; FIG. 12). Moreover, consistent with the predicted function of GmKIX8-1 in repressing meristemoid cell division, the increased cotyledon size in the gmkix8-1 null mutants was due to increased cell number rather than to increased cell size (FIG. 1F,G; FIG. 10D,E). The increased cell number per area in gmkix8-1 cotyledons (FIG. 1G; FIG. 10E) suggests a compensation mechanism between cell proliferation and expansion (Horiguchi et al., 2006). Among the targets of the PPD/KIX/TPL repressor complex is the cell proliferation-specific D3-type family of cyclins (Ge et al., 2016; Baeke-landt et al., 2018). In this study, we found that GmCYCD3;1-10 is one of the downstream targets of the PPD-KIX repressor complex in soybean shoot tips but not GmCYCD3;2-17 or GmCYCD3;3-5 (FIG. 4C-E). However, as was the case in Arabidopsis AtCYCD3;2 and AtCYCD3;2 (Baekelandt et al., 2018), these GmCYCD3 genes may still be targets in other soybean tissues and/or developmental stages that were not examined here. In addition to D3-type cyclins, the PPD/KIX module, in association with the transcription factors MYC3/4, represses the expression of the transcriptional co-activator GIF1 (Liu et al., 2020). The KIX-PPD-MYC-GIF1 module was implicated in the control of Arabidopsis seed size by restraining cell proliferation of the outer integument during both ovule and early seed developmental stages (Liu et al., 2020). GIF1 expression was upregulated in Arabidopsis and Medicago harboring mutations in PPD and/or KIX genes (Ge et al., 2016; Liu et al., 2020), and in soybean where PPD genes were targeted by microRNA (Ge et al., 2016).

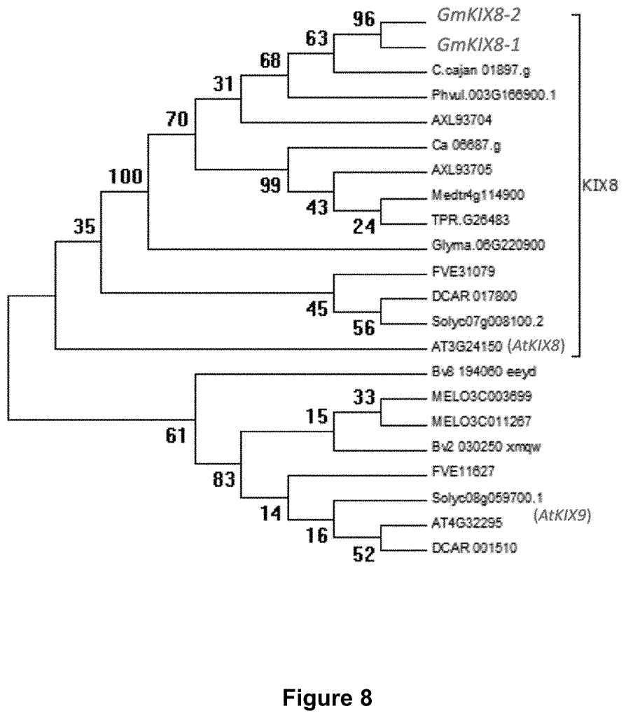

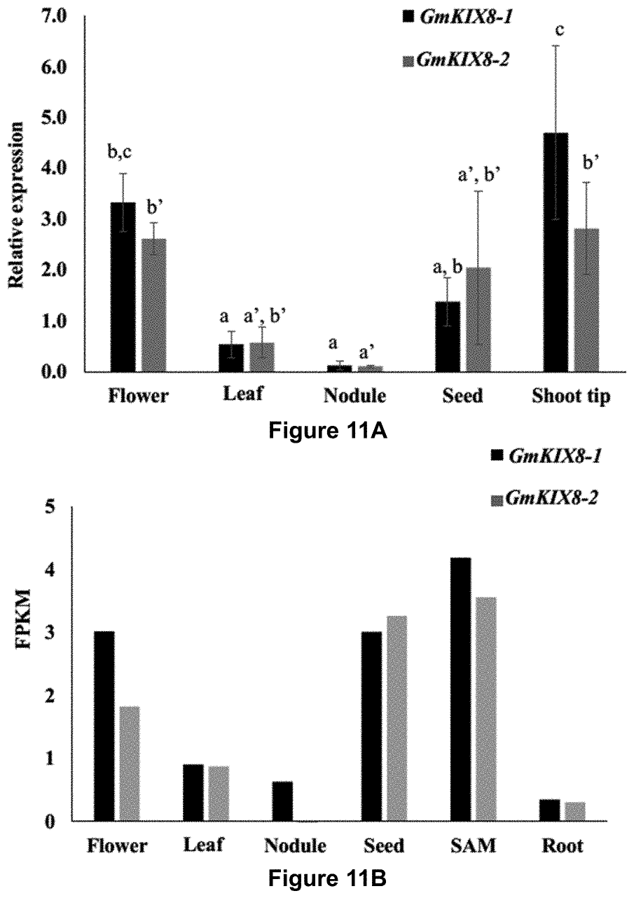

In contrast to leaf and seed size, no difference was observed in the root biomass produced by wild-type and gmkix8-1 plants (FIG. 1E). qRT-PCR results and publicly available soybean Gene Expression Atlas data showed that GmKIX8-1 is expressed across multiple organs and tissues, with the highest levels of expression in the shoot tips and seeds, and the lowest levels of expression in roots and nodules (FIG. 11). There is a strong correlation between the level of gene expression in a particular tissue and the severity of phenotype that is observed (i.e., mutant phenotype(s) is (are) stronger in tissues where the mutated gene is strongly expressed (Liao & Weng, 2015; Barbeira et al., 2018). The lack of root phenotype in the gmkix8-1 plants is consistent with this notion, that is, GmKIX8-1 is weakly expressed in roots compared to developing seeds and leaves (FIG. 11). Consistent with its highly duplicated genome, a second, paralogous GmKIX8 gene, GmKIX8-2 (Glyma.13G158300), is present in the soybean genome and shares 93.1% sequence identity with GmKIX8-1. Approximately 50% of gene paralogs in soybean were found to be differentially expressed and thus had undergone expression sub-functionalization (Roulin et al., 2013). Both GmKIX8-1 and GmKIX8-2 were expressed at comparable levels in various soybean tissues (FIG. 8; FIG. 11), indicating no apparent tissue-specific expression sub-functionalization of the GmKIX paralogs. It is believed that regulation of root size has not been attributed to the PPD-KIX-TPL repressor complex in plants. However, the functional redundancy between GmKIX8-1 and GmKIX8-2 in controlling organ size in soybean, including roots, can be addressed in the future by phenotyping single and double GmKIX8 mutants.

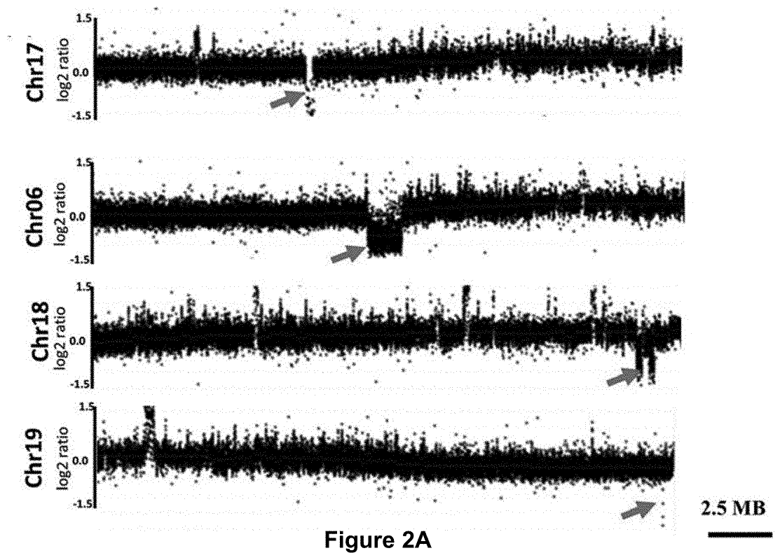

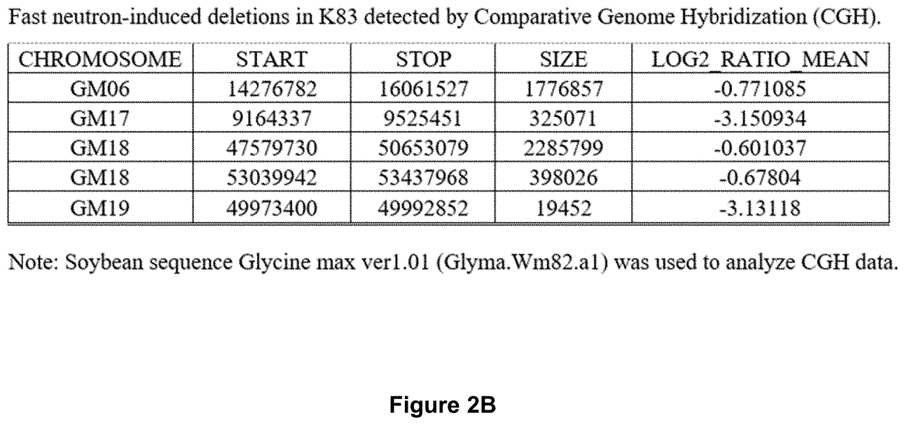

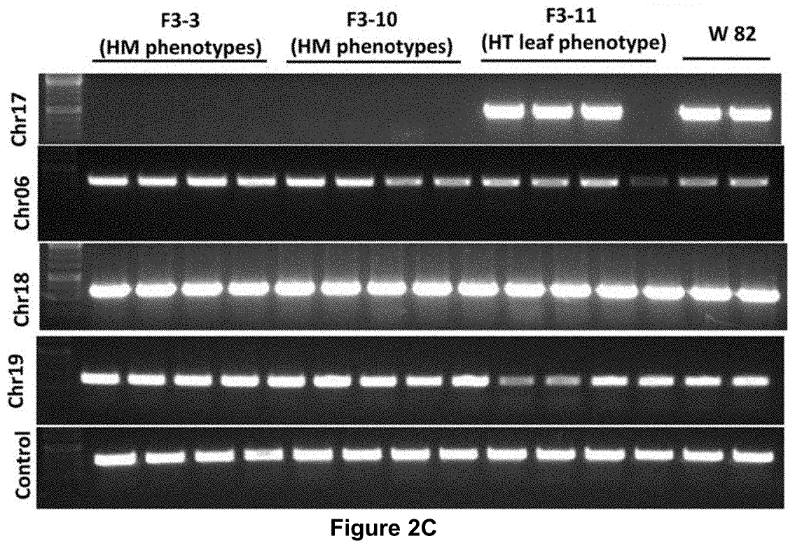

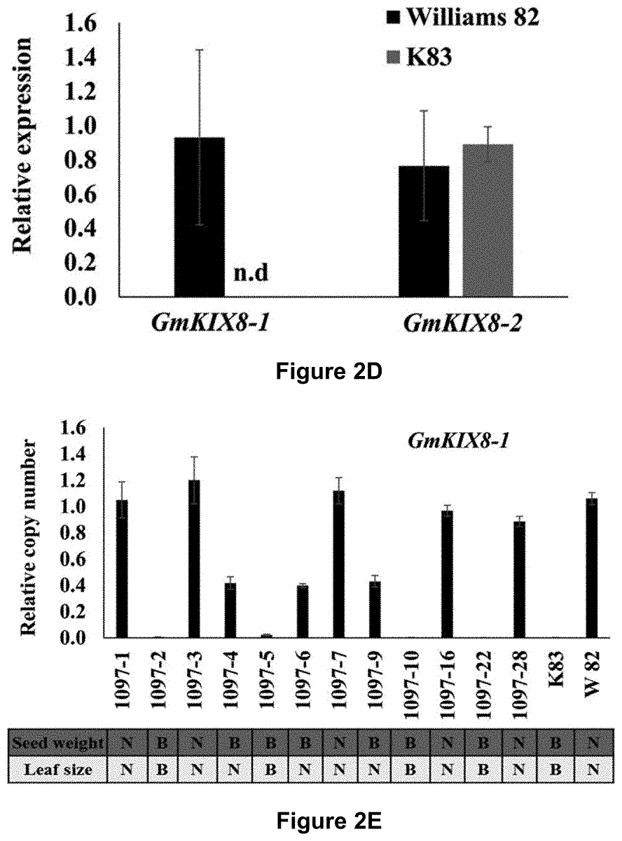

Maternal effects have been shown to be one of the major factors controlling seed size (Dilkes & Comai, 2004; Li et al., 2019). In M. truncatula, a mono-recessive mutant in BIG SEEDS1 (bsl-1), an orthologue of the AtPPD gene, produced large, heavy seeds. The increased seed size phenotype was shown to be controlled by the maternal genotype based on the sizes of F1 seeds derived from reciprocal crosses (Ge et al., 2016). However, in this study, a haplo-insufficient phenotype was observed for seed size, as indicated by our data, in which heterozygous and homozygous gmkix8-1 mutants produced big seeds (FIG. 2D; FIG. 3G; FIG. 10H; Table 1).

TABLE 1
GmKIX8-1 Genotype, seed size (100 seed weight)
and leaf phenotype of BC₁F₃ plants derived
from K83 X Williams 82 genetic crosses.
Data from two independent BC₁F₂ families
(1097 and 1100) are shown.

| | | Phenotype | | |
|---|---|---|---|---|
| | | 100 seed | Leaf | GmKIX8-1 |
| Generation | LineNumber | weight (g) | Phenotype | Genotype* |
| BC₁F₂ | 1097 | 24.0 | Normal | Heterozygous |
| (1097) | | | | |
| BC₁F₃ | 1097-1 | 14.6 | Normal | Wild-type |
| (1097) | 1097-2 | 18.0 | Big | Homozygous |
| | 1097-3 | 14.6 | Normal | Wild-type |
| | 1097-4 | 19.6 | Normal | Heterozygous |
| | 1097-5 | 19.9 | Big | Homozygous |
| | 1097-6 | 20.9 | Normal | Heterozygous |
| | 1097-7 | 14.5 | Normal | Wild-type |
| | 1097-9 | 17.2 | Normal | Heterozygous |
| | 1097-10 | 18.7 | Big | Homozygous |
| | 1097-16 | 12.4 | Normal | Wild-type |
| | 1097-28 | 14.4 | Normal | Wild-type |
| | 1097-22 | 19.5 | Big | Homozygous |
| BC₁F₂ | 1100 | 24.0 | Normal | Heterozygous |
| (1100) | | | | |
| BC1F3 | 1100-1 | 17.5 | Normal | Heterozygous |
| (1100) | 1100-7 | 19.0 | Big | Homozygous |
| | 1100-8 | 21.8 | Normal | Heterozygous |
| | 1100-10 | 18.3 | Normal | Heterozygous |
| | 1100-11 | 19.0 | Big | Homozygous |
| | 1100-14 | 19.8 | Normal | Heterozygous |
| | 1100-18 | 15.5 | Normal | Wild-type |
| | 1100-23 | 19.9 | Big | Homozygous |
| | 1100-24 | 18.4 | Big | Homozygous |
| | 1100-31 | 19.4 | Big | Homozygous |
| | 1100-33 | 15.2 | Normal | Wild-type |
| | 1100-44 | 15.1 | Normal | Wild-type |
| | 1100-50 | 19.9 | Big | Homozygous |

*Genotyping was done using qPCR Copy Number analysis.

Haplo-insufficiency phenotypes are usually associated with heterozygotes for mutations in transcription factors and components of signal transduction in human, mouse, Drosophila, Arabidopsis, and maize (Birchler et al., 2001; Seid-man & Seidman, 2002; Pillitteri et al., 2007; Boell et al., 2013; Yuan et al., 2014). In Arabidopsis, an E3 ubiquitin ligase, BIG BROTHER (BB) exerts dosage-dependent negative control on growth and is a limiting component of the organ-size checkpoint in flowers and the stem (Disch et al., 2006). Homozygous bb-1 mutants produce larger petals and sepals, thicker stems and larger leaves, whereas heterozygous plants have enlarged petals and sepals, and thicker stems, but produced wild-type leaves (Disch et al., 2006). Likewise, the homozygous soybean gmkix8-1 mutants produced large seeds, cotyledons and leaves, while heterozygous plants produced large seeds but the leaves were comparable in size to those of the wild-type (FIG. 1A-C; FIG. 2D; FIG. 3C-G; FIG. 10H). A likely explanation for the observed haplo-insufficiency of GmKIX8-1 in determining seed size is that cell proliferation in developing seeds is largely regulated by the PPD/KIX/TPL repressor complex and therefore is more responsive to gene dosage effects. By contrast, in addition to the PPD/KIX/TPL repressor complex, leaf size in soybean is likely controlled by other redundant transcription regulator(s) and/or physiological factors that modulate cell proliferation and cell size. Although data clearly demonstrated haplo-insufficiency of GmKIX8-1 in controlling seed size in soybean, it remains to be determined whether seed size is also maternally controlled by the GmKIX8-1 genotype, as was the case with AtKIX8/9 in Arabidopsis (Liu et al., 2020) and BIG SEEDS 1 in Medicago, by reciprocal genetic crosses. Future experiments on the phenotypic effects of GmKIX8-1 overexpression can also shed additional light on the dosage-dependent control of soybean organ size by GmKIX8-1.

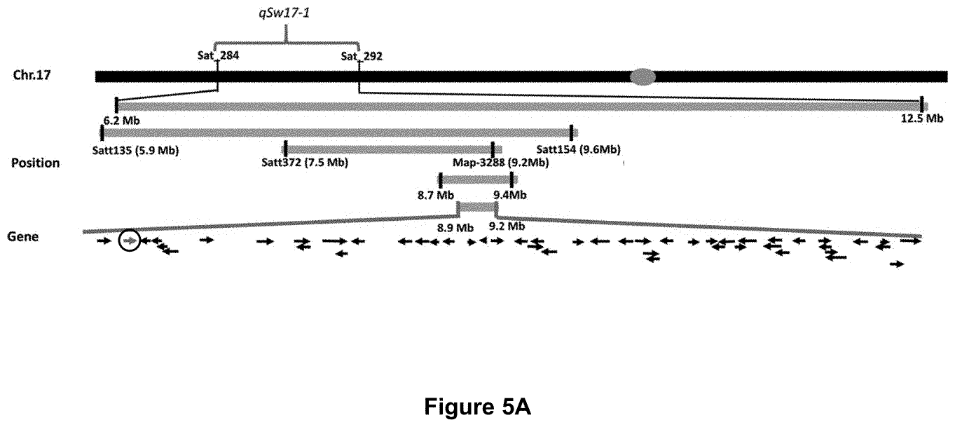

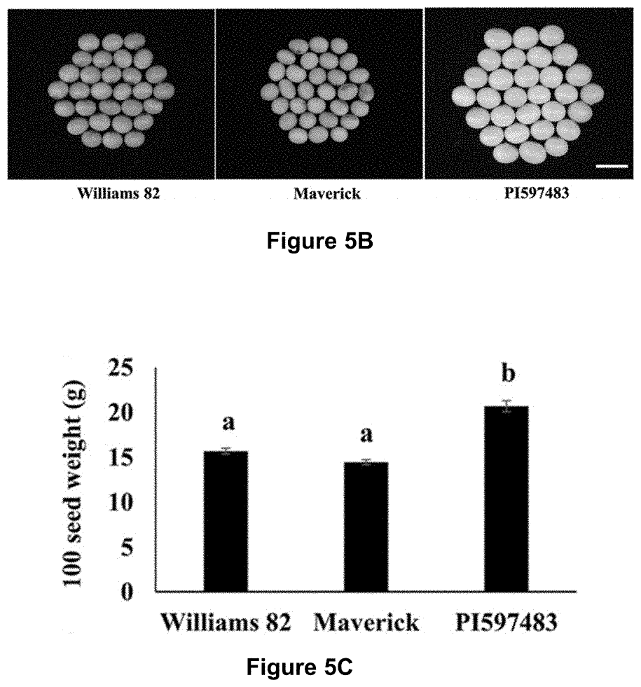

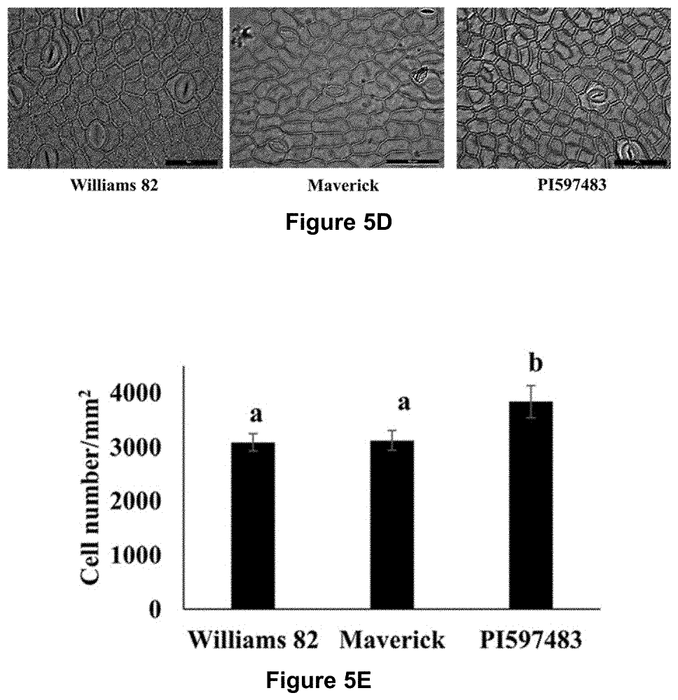

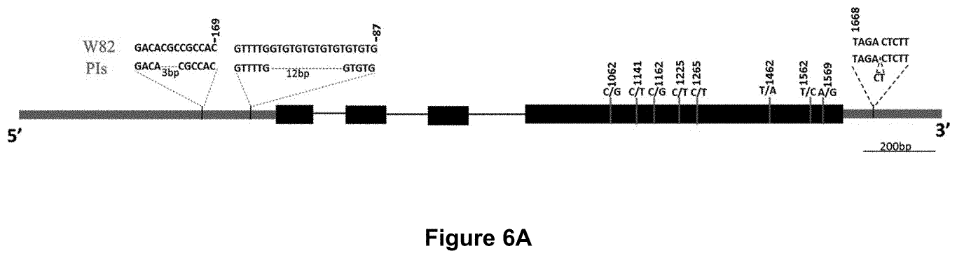

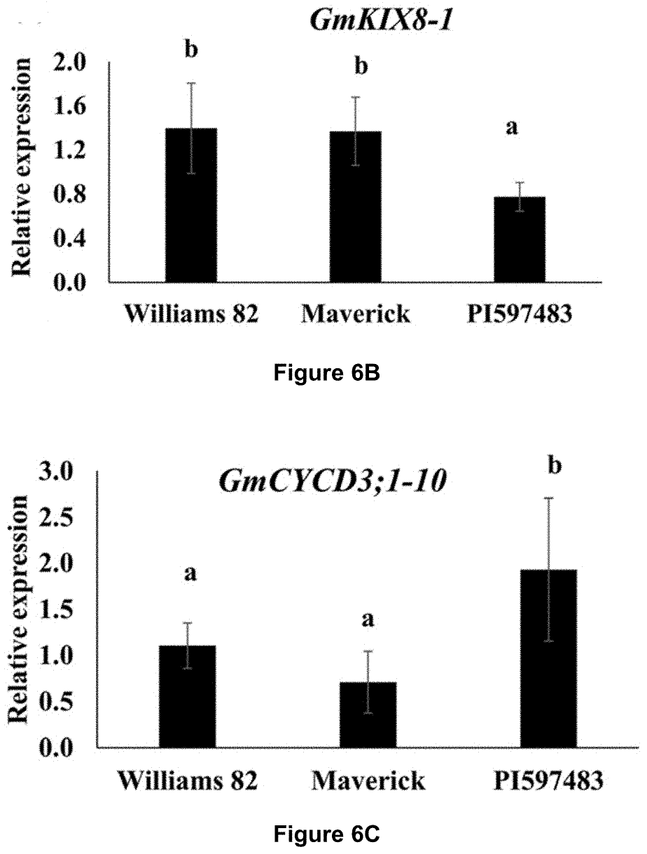

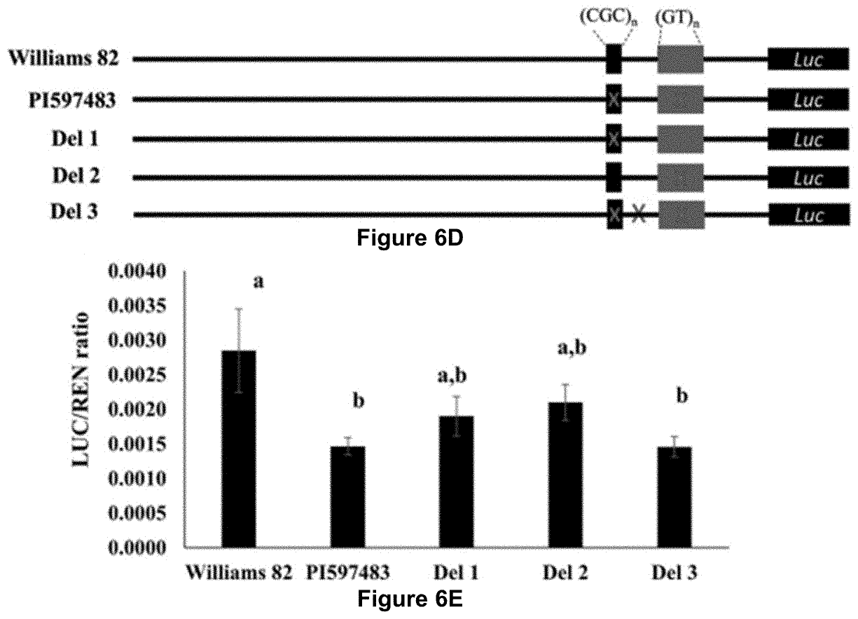

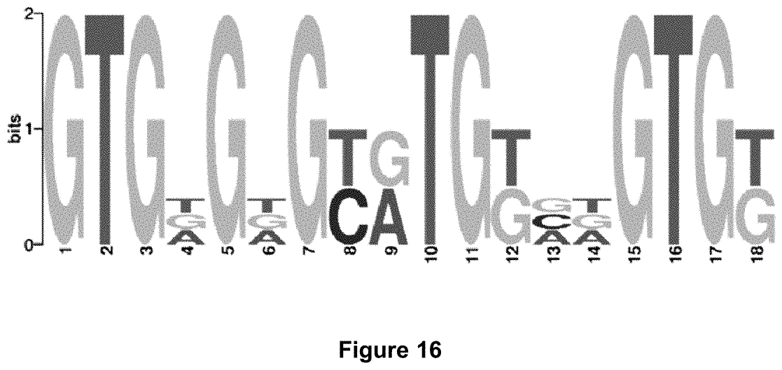

qSw17-1 is a well-known QTL associated with seed weight in soybean (Hoeck et al., 2003; Panthee et al., 2005; Liu et al., 2007, 2013, 2018; Teng et al., 2008; Kim et al., 2010; Kato et al., 2014; Zhou et al., 2015; Yan et al., 2017; Karikari et al., 2019). Considerable effort has been invested in fine-mapping the QTL, and so far the smallest region identified corresponded to a c. 700 kb region on chromosome 17 (FIG. 5A) (Jing et al., 2018; Zhang et al., 2018). The slow progress in identifying causative genes for mapped QTLs can be attributed to the bottleneck created by the low genetic diversity and complex genome structure of cultivated soybean. Since the deleted region in the FN mutant K83 encoding GmKIX8-1 overlaps with the mapped qSw17-1, it was hypothesized that polymorphism(s) in GmKIX8-1 could be responsible for the increased seed weight associated with QTL qSw17-1. Indeed, sequencing of GmKIX8-1 coding and promoter regions in three big-seeded soybean PI lines harboring qSw17-1 identified common deletions in the promoter region that negatively affected transcriptional expression of GmKIX8-1 (FIG. 6A,B). Specifically, reduced GmKIX8-1 expression is likely due to the deletion of a GT-dinucleotide repeat predicted to be part of a cis-regulatory-element (CRE) for transcription factor(s) binding (FIG. 16). GT tandem repeat CREs are widely found in plant promoter regions and are involved in various developmental and stress response pathways in plants (Zhou, 1999; Kaplan-Levy et al., 2012). As previously discussed, we observed haplo-insufficiency of GmKIX8-1 in determining seed size but not leaf size. The reduced expression of GmKIX8-1 in the big-seeded PIs (FIG. 6B,E) essentially translates to reduced gene dosage, and as such, resulted in the observed increase in seed size in these PIs. Although no increase in leaf size was observed, GmCYCD3;1-10 was upregulated in the shoot tips of the big-seeded PIs (FIG. 6C), suggesting that other leaf phenotypes which were not examined in this study (e.g. cell number) may still be affected in these genotypes. Lastly, traits in crop domestication and improvement are often caused by mutations in CREs that result in subtle alterations in expression and avoid pleotropic effects (Swinnen et al., 2016)). As exemplified by the CRE deletions in the GmKIX8-1 promoter, modulating the expression of genes in the PPD-KIX pathway by targeting CREs by gene editing can be a feasible approach to improving yield in dicotyledonous crop plants.

### Description

In the following passages, different aspects of the invention are defined in more detail. Each aspect so defined may be combined with any other aspect or aspects unless clearly indicated to the contrary.

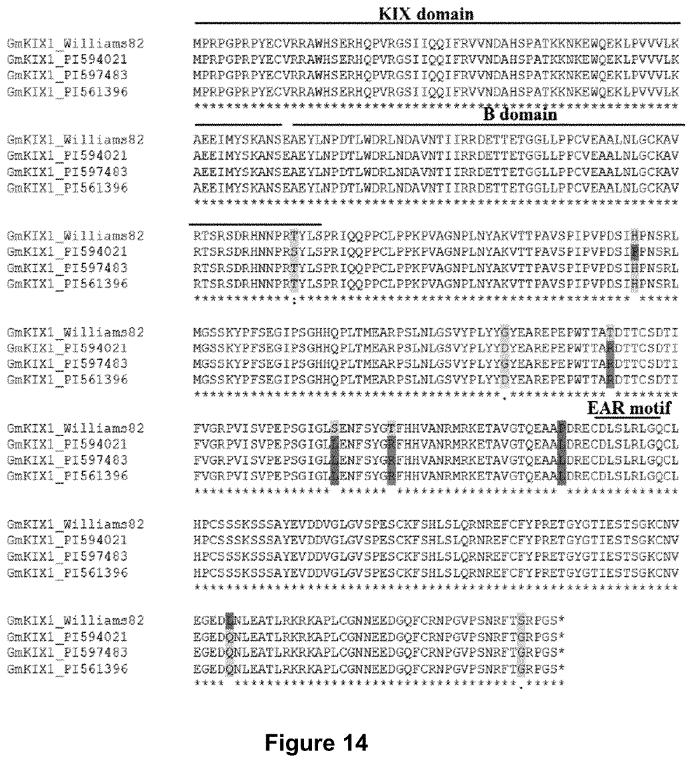

### Soybean GmKIX Genes

Whereas for simplicity, reference is made to the soybean GmKIX8-1 gene Glyma.17G112800 throughout this disclosure, it is understood that in any embodiment of this disclosure in which reference is made to the soybean GmKIX8-1 gene Glyma.17G112800, where applicable, other corresponding embodiments drawn to genes homologous to GmKIX8-1 are explicitly provided for (collectively, including GmKIX8-1, referred to herein as soybean GmKIX genes). Exemplary GmKIX genes homologous to GmKIX8-2 include GmKIX8-2 (Glyma.13G158300; SEQ ID NO: 82) which encodes the protein of amino acid sequence SEQ ID NO: 85 and Glyma.06G220900 (SEQ ID NO: 83) which encodes the protein of amino acid sequence SEQ ID NO: 86. In certain embodiments, a homologous GmKIX gene encodes for a protein having at least 60%, 65%, 70%, 80%, 85%, 90%, 95%, 96%, 97%, 98%, or 99% identity with the GmKIX8-1 protein of SEQ ID NO: 84. For example, GmKIX8-2 protein shares over 90% amino acid identity with GmKIX8-1 protein and the protein encoded by Glyma.06G220900 shares over 60% amino acid identity with GmKIX8-1 protein. In certain embodiments, a homologous GmKIX gene encodes for a protein having at least 60%, 65%, 70%, 80%, 85%, 90%, 95%, 96%, 97%, 98%, or 99% identity with the GmKIX8-2 protein of SEQ ID NO: 85. In certain embodiments, a homologous GmKIX gene encodes for a protein having at least 60%, 65%, 70%, 80%, 85%, 90%, 95%, 96%, 97%, 98%, or 99% identity with the protein of SEQ ID NO: 86. In certain embodiments, a soybean GmKIX protein amino acid sequence comprises a KIX domain, B domain, and/or EAR motif as shown in FIG. 14.

### Obtaining a Soybean Plant Comprising in its Genome at Least One Large-Seed Phenotype Genetic Locus.

Provided for herein is a method for obtaining a soybean plant comprising in its genome at least one genetic locus that comprises a genotype associated with a large-seed phenotype. In certain embodiments, the large-seed phenotype genetic locus is a genetic locus comprising the soybean GmKIX8-1 gene Glyma.17G112800 and/or a homologous GmKIX gene and any embodiments thereof as described in detail elsewhere herein. In certain embodiments, the method comprises the steps of (a) genotyping one or more soybean plants with respect to a genetic locus comprising the soybean GmKIX8-1 gene and/or a homologous GmKIX gene and (b) selecting based on said genotyping of said genetic locus a soybean plant comprising a genotype associated with a large-seed phenotype. In certain embodiments, the method comprises the steps of (a) genotyping one or more soybean plants with respect to a genetic locus comprising the soybean GmKIX8-1 gene and (b) selecting based on said genotyping of said genetic locus a soybean plant comprising a genotype associated with a large-seed phenotype. In certain embodiments, the selected soybean plant or a progeny thereof exhibits the large-seed phenotype.

In certain embodiments, the genotype, haplotype, polymorphic allele, allelic state, single nucleotide polymorphism, genetically edited variant, etc., associated with the large-seed phenotype is selected from any of the embodiments as described in detail elsewhere herein. In certain embodiments, the selected soybean plant exhibits larger seeds in comparison to a control soybean plant that does not comprises the genotype, haplotype, polymorphic allele, allelic state, single nucleotide polymorphism, genetically edited variant, etc., associated with the large-seed phenotype. In certain embodiments, the selected soybean plant exhibits larger seeds in comparison to a soybean plant that is not considered to have a large-seed phenotype. In certain embodiments, the selected soybean plant exhibits a large-seed phenotype. In certain embodiments, a progeny of the selected soybean plant exhibits larger seeds in comparison to a control soybean plant that does not comprises the genotype, haplotype, polymorphic allele, allelic state, single nucleotide polymorphism, genetically edited variant, etc., associated with the large-seed phenotype. In certain embodiments, a progeny of the selected soybean plant exhibits larger seeds in comparison to a soybean plant that is not considered to have a large-seed phenotype. In certain embodiments, a progeny of the selected soybean plant exhibits a large-seed phenotype.

Once selected, a soybean plant comprising in its genome a large-seed phenotype genetic locus can used for breeding and/or in a breeding program to produce progeny comprising in their genomes the large-seed phenotype genetic locus. In certain embodiments, it is useful to use a soybean plant comprising in its genome a large-seed phenotype genetic locus to introduce the locus into germplasm that does not comprise the large-seed phenotype genetic locus. Thus in certain embodiments, the selected soybean plant having in its genome a large-seed phenotype genetic locus is crossed with a soybean plant that does not have a large-seed phenotype genetic locus to produce a progeny soybean plant comprising in its genome at least one large-seed phenotype genetic locus. In certain embodiments, the progeny plant exhibits a large-seed phenotype. Because soybean is planted as a commercial crop, it is desirable to produce a population of soybean plants comprising a plurality of soybean plants comprising in their genomes the large-seed phenotype genetic locus. Thus in certain embodiments, a soybean plant having in its genome a large-seed phenotype genetic locus is crossed with a soybean plant that does not have a large-seed phenotype genetic locus to produce a population of soybean plants comprising in their genomes at least one large-seed phenotype genetic locus. The production of such a population of soybean plants include the use of soybean plant having in their genome an introgressed large-seed phenotype genetic locus as described elsewhere herein and thus any of these embodiments apply to introgressed plants. In certain embodiments, the population of soybean plants produced comprises a plurality of soybean plants that exhibit a large-seed phenotype. It is understood that not all of the soybean plants in a population, depending on how the population is produced and/or determined, will have inherited the large-seed phenotype genetic locus and/or will exhibit a large-seed phenotype. In certain embodiments, at least 50%, at least 60%, at least 70%, at least 75%, at least 80%, at least 85%, at least 90%, at least 95%, at least 96%, at least 97%, at least 98%, at least 99%, or 100% of the soybean plants in the population of soybean plants produced exhibit a large-seed phenotype.

In certain embodiments, the large-seed phenotype genetic locus is a genomic region between any of about 1 Mb, 500 kb, 100 kb, 50 kb, or 10 kb telomere proximal and any of about 1 Mb, 500 kb, 100 kb, 50 kb, or 10 kb centromere proximal of a soybean GmKIX gene.

In certain embodiments, the genetic locus comprising the soybean GmKIX8-1 gene is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the soybean GmKIX8-1 gene Glyma.17G112800. In certain embodiments, the genetic locus comprises Gm17:8899818-89100577. This includes 5 kb 5′ upstream, gene, and 2 kb downstream sequences of GmKIX8-1 based on Wm82.a4.v1 annotation. In certain embodiments, the genetic locus comprises Gm17:8902818-8909577. this includes 2 kb promoter, gene sequence and 1 kb downstream sequences of GmKIX8-1 based on Wm82.a4.v1 annotation. In certain embodiments, the genetic locus consists of the soybean GmKIX8-1 gene Glyma.17G112800. In certain embodiments, the genetic locus comprises or consists of the promoter region, 5′ UTR, 3′ UTR, the protein coding region, an intron, and/or an exon of the GmKIX8-1 gene. In certain embodiments, the genetic locus comprises or consists of a CIS-regulatory element (CRE) for transcription factor binding of the GmKIX8-1 gene.

In certain embodiments, the genetic locus comprising the soybean GmKIX8-2 gene Glyma.13G158300 is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the soybean GmKIX8-2 gene Glyma.13G158300. In certain embodiments, the genetic locus comprises Gm13:26760510-26770918. This includes 5 kb 5′ upstream, gene, and 2 kb downstream sequences of GmKIX8-2 based on Wm82.a4.v1 annotation. In certain embodiments, the genetic locus comprises Gm13:26761510-26767918. this includes 2 kb promoter, gene sequence and 1 kb downstream sequences of GmKIX8-2 based on Wm82.a4.v1 annotation. In certain embodiments, the genetic locus consists of the soybean GmKIX8-2 gene Glyma.13G158300. In certain embodiments, the genetic locus comprises or consists of the promoter region, 5′ UTR, 3′ UTR, the protein coding region, an intron, and/or an exon of the GmKIX8-2 gene. In certain embodiments, the genetic locus comprises or consists of a CIS-regulatory element (CRE) for transcription factor binding of the GmKIX8-2 gene.

In certain embodiments, the genetic locus comprising the soybean Glyma.06G220900 gene is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the soybean Glyma.06G220900 gene. In certain embodiments, the genetic locus comprises Gm06:24615419-24625694. This includes 5 kb 5′ upstream, gene, and 2 kb downstream sequences of Glyma.06G220900 based on Wm82.a4.v1 annotation. In certain embodiments, the genetic locus comprises Gm06:24618418-24624694. this includes 2 kb promoter, gene sequence and 1 kb downstream sequences of GmKIX8-1 based on Wm82.a4.v1 annotation. In certain embodiments, the genetic locus consists of the soybean Glyma.06G220900 gene. In certain embodiments, the genetic locus comprises or consists of the promoter region, 5′ UTR, 3′ UTR, the protein coding region, an intron, and/or an exon of the Glyma.06G220900 gene. In certain embodiments, the genetic locus comprises or consists of a CIS-regulatory element (CRE) for transcription factor binding of the Glyma.06G220900 gene.

In certain embodiments, genotyping is done with a SSR marker primer pair comprising the sequences of F-KIX-SSR-GRIN GAGTGAGTGAGCACTGTGTTGTG (SEQ ID NO: 53) and R-KIX-SSR-GRIN ACCAAAACCGCCCCAAGACACTC (SEQ ID NO: 54), a primer pair comprising the sequences of F-KIX1CRISPGenotype TTCTCTCGCTACTCCTCCTACC (SEQ ID NO: 47) and R-KIX1CRISPGenotype GTACTCTGCCTAAGCAACAACCA (SEQ ID NO: 48), a primer pair comprising the sequences of F-KIX2CRISPGenotype GAGTGAGCGAGTGAGCACTGCC (SEQ ID NO: 49) and F-KIX2CRISPGenotype CAAATTCCGCAAGCATTTTGTG (SEQ ID NO: 50), or a primer pair comprising the sequences of F-KIX1-GRIN GGTACGGACATAGTTCACGATCCC (SEQ ID NO: 55) and R-KIX1-GRIN GATTCCTTGTCCATATCCATTATCC (SEQ ID NO: 56).

In certain embodiments, the genotype associated with the large-seed phenotype is a deletion of or within the soybean GmKIX8-1 gene Glyma.17G112800 and/or a homologous GmKIX gene. In certain embodiments, the genotype associated with the large-seed phenotype is a deletion within the protein coding region and/or the promoter region of the soybean GmKIX8-1 gene Glyma.17G112800 and/or a homologous GmKIX gene. In certain embodiments, the genotype associated with the large-seed phenotype is a deletion within the promoter region of the soybean GmKIX8-1 gene Glyma.17G112800 and/or a homologous GmKIX gene. For example, in certain embodiments, a deletion in the promoter region comprises a deletion in or of a CIS-regulatory element (CRE) for transcription factor binding. In certain embodiments, a promoter region deletion comprises a deletion of the tandem repeated sequence CGC located at −177 to −174 relative to the GmKIX8-1 gene ATG start site. In certain embodiments, a promoter region deletion comprises a deletion of the tandem repeated sequence GT located at −104 to −92 relative to the GmKIX8-1 gene ATG start site. And, in certain embodiments, a promoter region deletion comprises both of the aforementioned deletions.

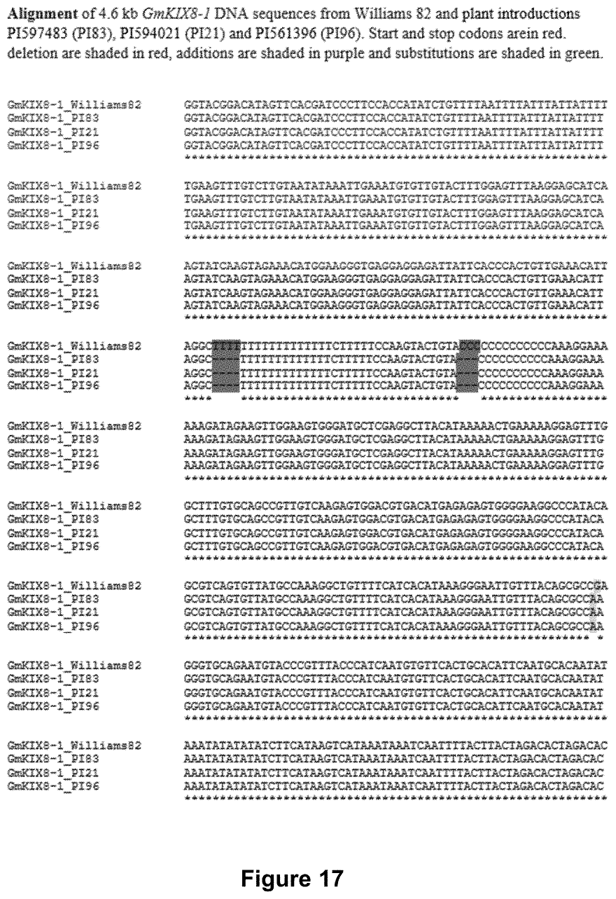

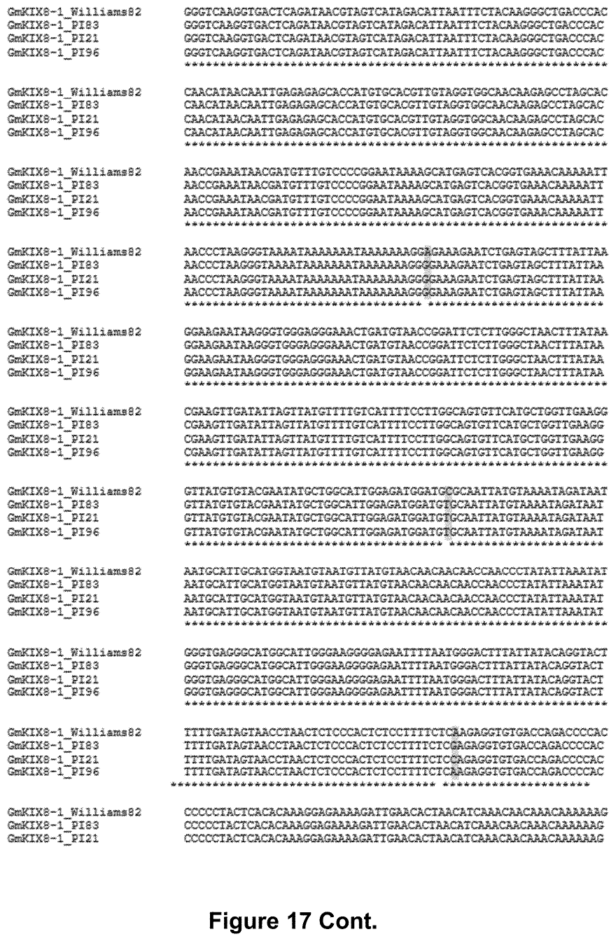

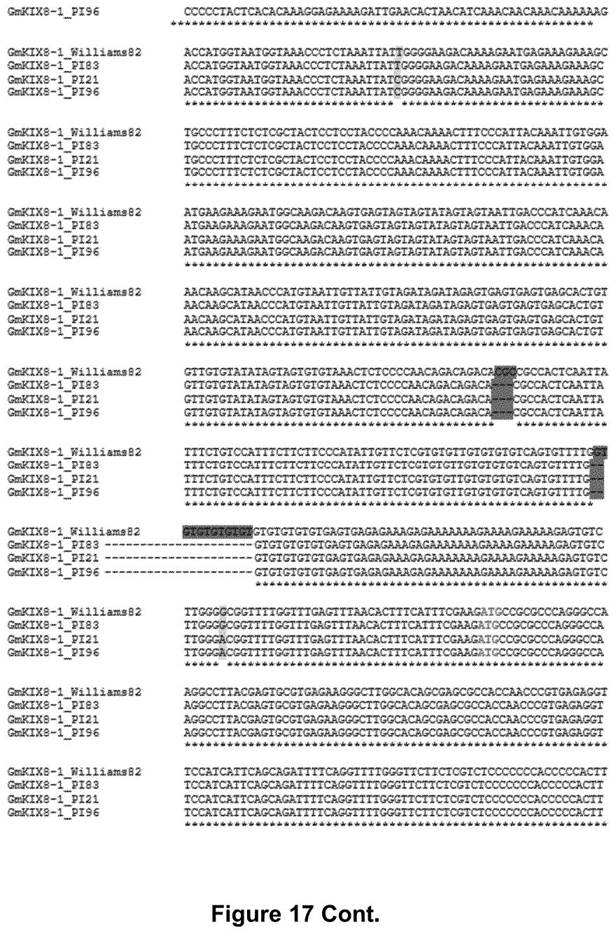

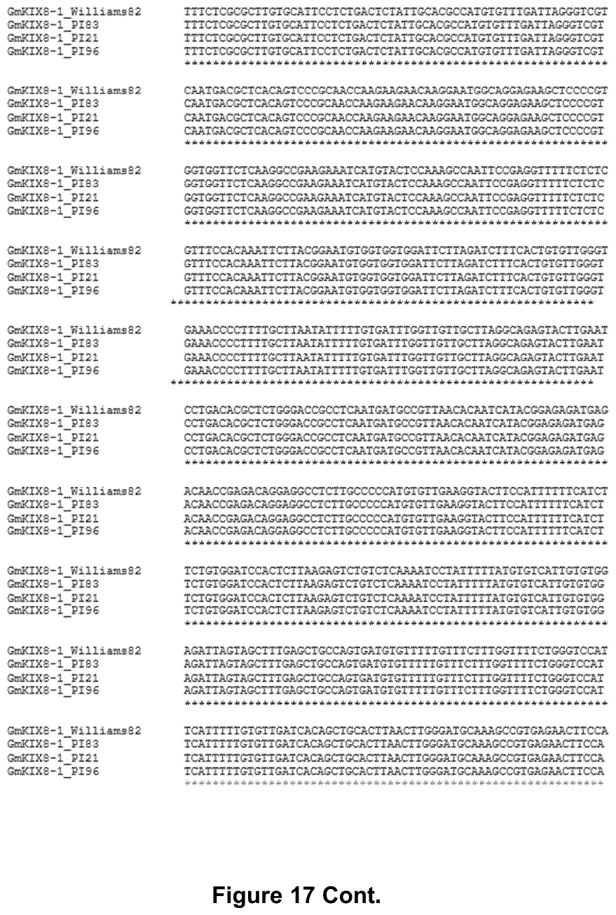

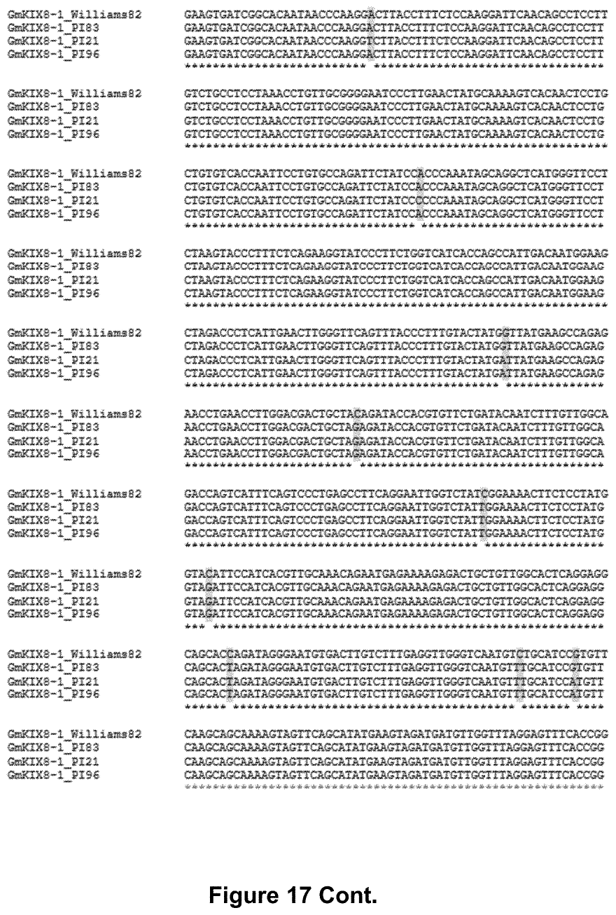

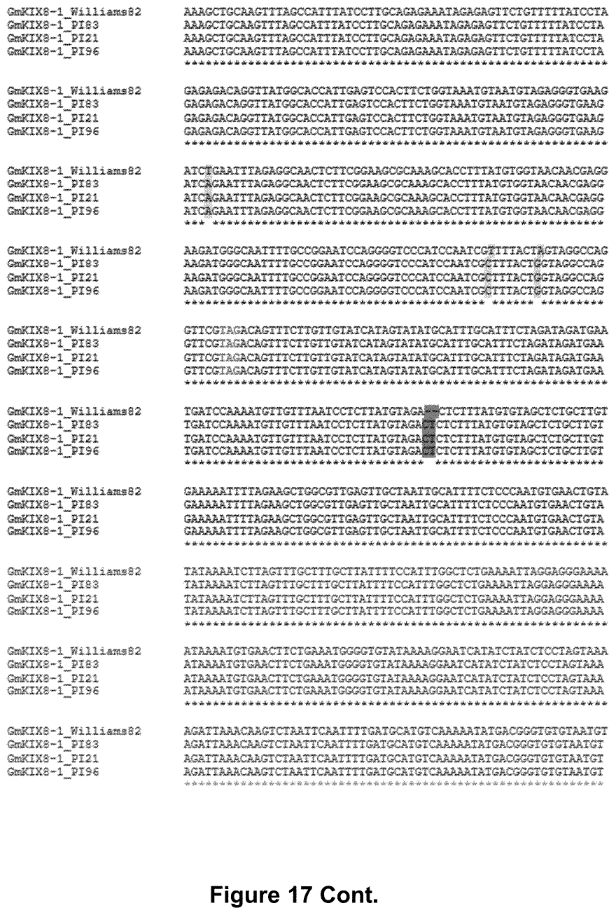

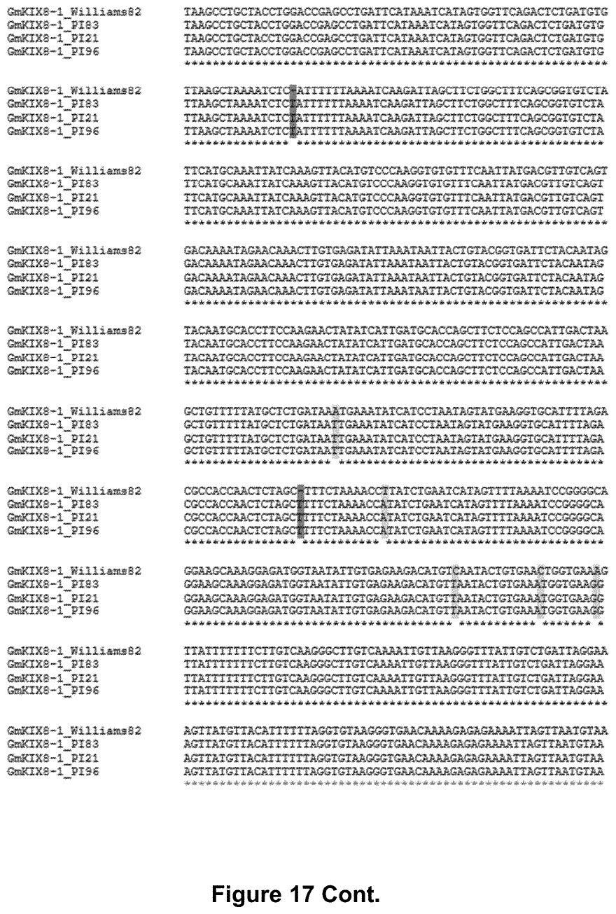

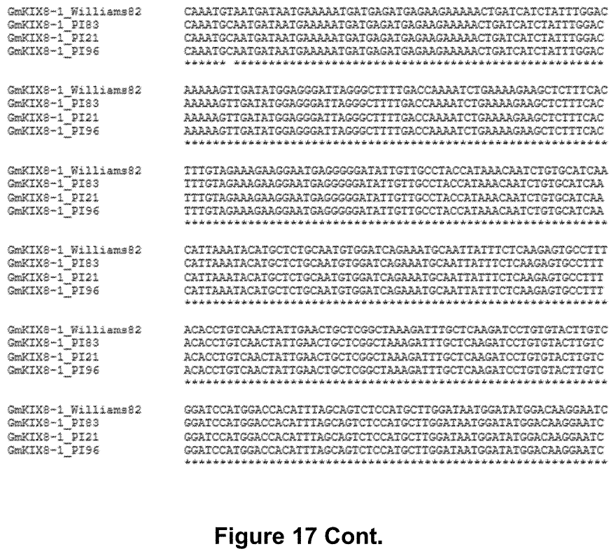

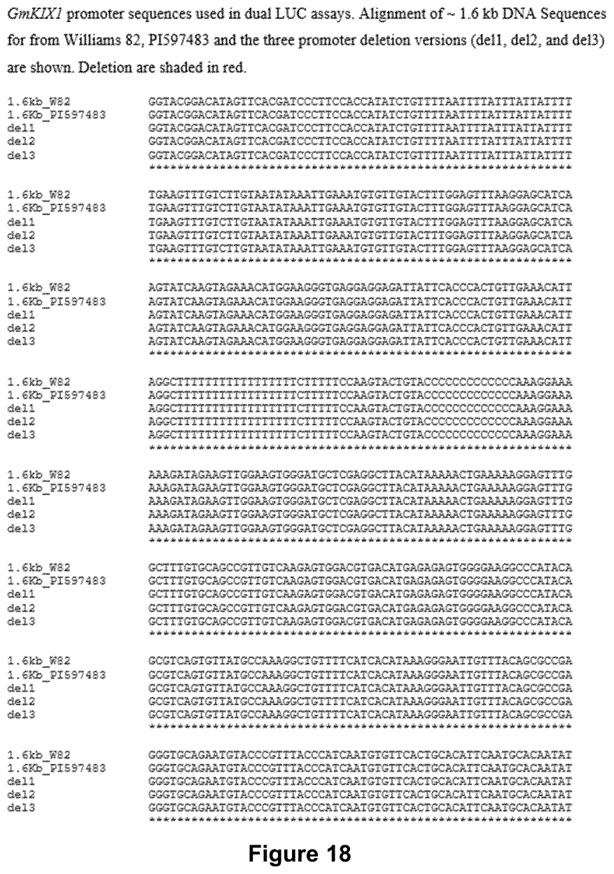

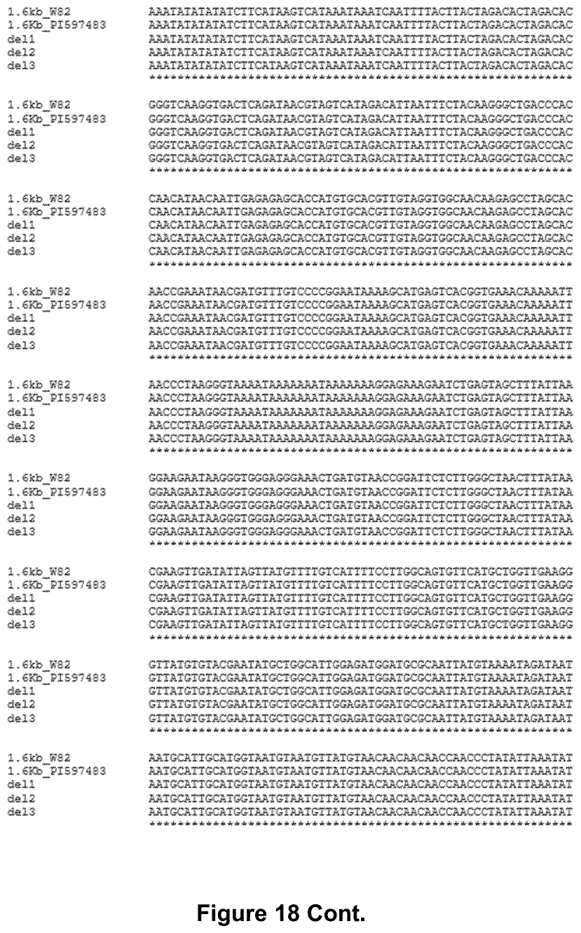

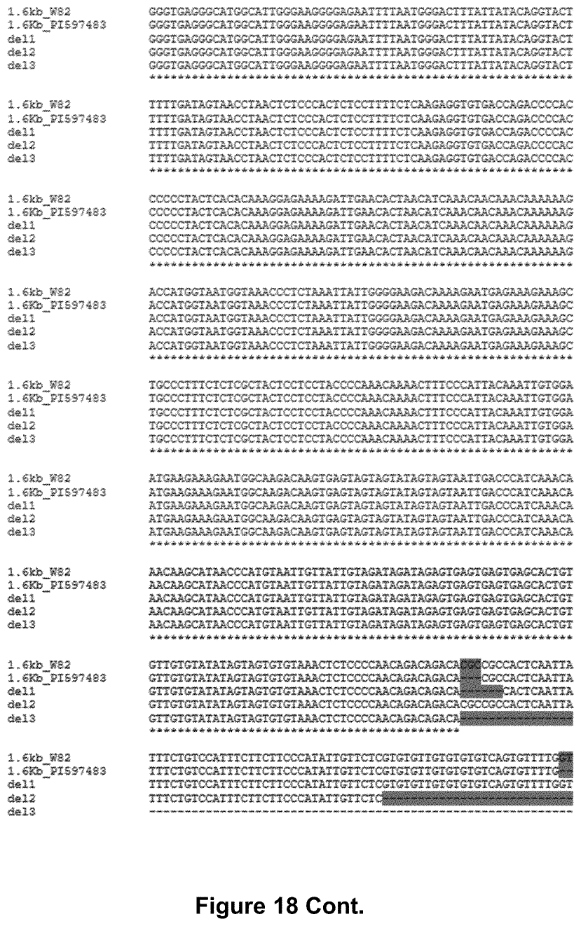

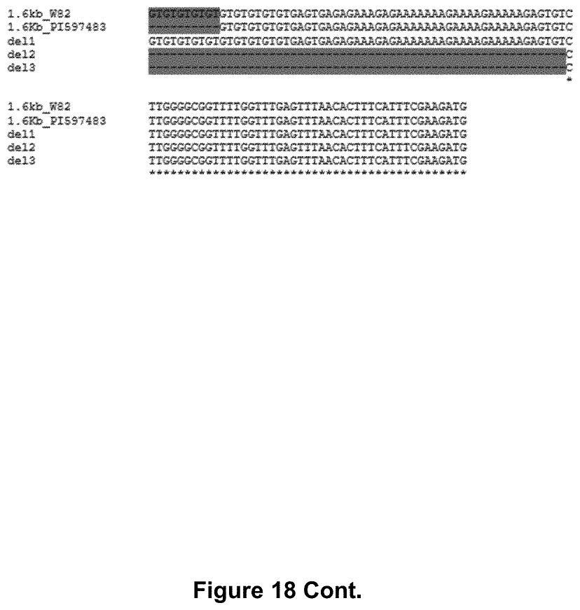

In certain embodiments, the genotype associated with the large-seed phenotype comprises at least one allele associated with the large-seed phenotype identified in the alignment of soybean GmKIX8-1 genes in FIG. 17. In certain embodiments, the genotype associated with the large seed phenotype comprises at least one of the two 3′ most deletions in the promoter region as shown in the alignment in FIG. 17. In certain embodiments, the genotype associated with the large seed phenotype comprises at least the two 3′ most deletions in the promoter region as shown in the alignment of FIG. 17. Examples of such deletions are shown in FIG. 18.

In certain embodiments, the genetic locus comprising a GmKIX gene comprising a genotype associated with a large-seed phenotype has been gene edited. In certain embodiments, the genetic locus comprising the GmKIX8-1 gene Glyma.17G112800I comprising a genotype associated with a large-seed phenotype has been gene edited, such as for example, as described elsewhere herein.

While the detection of a single allele having an allelic state associated with a large-seed phenotype genetic locus can be predictive of a large-seed phenotype, in some cases it is useful to determine the allelic state of additional genetic markers, such as to strengthen the prediction. In any embodiments herein involving the genotyping of an allele, the embodiments also provide for genotyping for/determining, and optionally selecting/crossing/breeding a plant based thereon, the presence of a haplotype associated with the large-seed phenotype. Further, in some cases, it may be useful to determine whether a large-seed phenotype associated allele and/or locus is present in a germplasm that naturally contains the allele and/or locus or whether the allele and/or locus has been artificially introduced (as to produce a new, non-naturally occurring plant), such as by gene editing and/or introgression, into the germplasm. Thus in certain embodiments, the method further comprises genotyping for the presence of at least one additional marker. In some embodiments, the additional marker is associated with the large-seed phenotype. In some embodiments, the additional marker is linked to the genomic locus associated with a large-seed phenotype disclosed herein. In certain embodiments, the additional marker is not linked to the genomic locus associated with a large-seed phenotype.

Producing a Soybean Plant Through Introgression of a Genomic Region Associated with a Large-Seed Phenotype

Also provided herewith is unique soybean germplasm comprising an introgressed genomic region that is associated with a large-seed phenotype and methods of obtaining the same. Marker-assisted introgression involves the transfer of a chromosomal region, defined by one or more markers, from one germplasm to a second germplasm. Offspring of a cross that contain the introgressed genomic region can be identified by the combination of markers characteristic of the desired introgressed genomic region from a first germplasm (i.e., such as a large-seed phenotype germplasm) and both linked and unlinked markers characteristic of the desired genetic background of a second germplasm (i.e., a non-large-seed phenotype germplasm).

Certain embodiments of this disclosure provide for a method for producing a soybean plant comprising in its genome an introgressed genetic locus comprising a genotype associated with a large-seed phenotype comprising the steps of: a) crossing a first soybean plant with a genotype associated with a large-seed phenotype in a first polymorphic genetic locus comprising a soybean GmKIX gene with a second soybean plant comprising a genotype not associated with a large-seed phenotype in the polymorphic genetic locus comprising soybean GmKIX gene and at least one second polymorphic locus that is linked to the genetic locus comprising soybean GmKIX gene and that is not present in said first soybean plant to obtain a population segregating for the large-seed phenotype polymorphic locus and said linked second polymorphic locus; b) genotyping for the presence of at least two polymorphic nucleic acids in at least one soybean plant from said population, wherein a first polymorphic nucleic acid is located in said genetic locus comprising soybean GmKIX gene and wherein a second polymorphic amino acid is the linked second polymorphic locus not present in said first soybean plant; and c) selecting a soybean plant comprising a genotype associated with the large-seed phenotype and the at least one linked marker found in said second soybean plant that does not comprise a large-seed phenotype locus but not found in said first soybean plant, thereby obtaining a soybean plant comprising in its genome an introgressed large-seed phenotype locus.

Certain embodiments of this disclosure provide for a method for producing a soybean plant comprising in its genome an introgressed genetic locus comprising a genotype associated with a large-seed phenotype comprising the steps of: a) crossing a first soybean plant with a genotype associated with a large-seed phenotype in a first polymorphic genetic locus comprising the soybean GmKIX8-1 gene Glyma.17G112800 with a second soybean plant comprising a genotype not associated with a large-seed phenotype in the polymorphic genetic locus comprising Glyma.17G112800 and at least one second polymorphic locus that is linked to the genetic locus comprising Glyma.17G112800 and that is not present in said first soybean plant to obtain a population segregating for the large-seed phenotype polymorphic locus and said linked second polymorphic locus; b) genotyping for the presence of at least two polymorphic nucleic acids in at least one soybean plant from said population, wherein a first polymorphic nucleic acid is located in said genetic locus comprising Glyma.17G112800 and wherein a second polymorphic amino acid is the linked second polymorphic locus not present in said first soybean plant; and c) selecting a soybean plant comprising a genotype associated with the large-seed phenotype and the at least one linked marker found in said second soybean plant that does not comprise a large-seed phenotype locus but not found in said first soybean plant, thereby obtaining a soybean plant comprising in its genome an introgressed large-seed phenotype locus.

In certain embodiments, the genetic locus comprising a genotype associated with the large-seed phenotype is a genomic region as described in detail above and disclosed elsewhere herein. In certain embodiments, the genotype associated with the large-seed phenotype is a genotype as described in detail above and disclosed elsewhere herein. In certain embodiments, genotyping is done with a SSR marker primer pair disclosed elsewhere herein.

In certain embodiments, the polymorphic genetic locus comprising a GmKIX gene comprising a genotype associated with a large-seed phenotype has been gene edited. In certain embodiments, the polymorphic genetic locus comprising the GmKIX8-1 gene Glyma.17G112800I comprising a genotype associated with a large-seed phenotype has been gene edited, such as for example, as described elsewhere herein.

In certain embodiments, the population of soybean plants produced comprises a plurality of soybean plants that exhibit the large-seed phenotype. In certain embodiments, second linked polymorphic locus is detected with a marker that is located within about 1 Mb, 500, 100, 40, 20, 10, or 5 kilobases (Kb) of a soybean GmKIX gene. In certain embodiments, second linked polymorphic locus is detected with a marker that is located within about 1 Mb, 500, 100, 40, 20, 10, or 5 kilobases (Kb) of Glyma.17G112800.

### Identification of Soybean Plants Comprising a Large-Seed Phenotype Associated Genotype

Provided herein is a method of identifying a soybean plant that comprises or does not comprise a genotype associated with a large-seed phenotype of this disclosure. In certain embodiments, the method comprises genotyping a soybean plant for the presence of or absence an allele in a genetic locus associated with a large-seed phenotype, wherein the genetic locus comprises a GmKIX gene, as disclosed in detail elsewhere herein. In certain embodiments, the method comprises genotyping a soybean plant for the presence of or absence an allele in a genetic locus associated with a large-seed phenotype, wherein the genetic locus comprises the GmKIX8-1 gene Glyma.17G112800, as disclosed in detail elsewhere herein. In certain embodiments, the method comprises denoting based on the genotyping that the soybean plant comprises or does not comprise a genotype associated with a large-seed phenotype. In certain embodiments, the method further comprises the step of selecting a denoted plant either comprising or not comprising a genotype associated with a large-seed phenotype from a population of plants. In certain embodiments, the identified and/or selected soybean plant comprising in its genome a large-seed phenotype genetic locus exhibits a large-seed phenotype.

In certain embodiments, the genetic locus comprising a genotype associated with the large-seed phenotype is a genomic region as described in detail above and disclosed elsewhere herein. In certain embodiments, the genotype associated with the large-seed phenotype is a genotype as described in detail above and disclosed elsewhere herein. In certain embodiments, genotyping is done with a SSR marker primer pair disclosed elsewhere herein.

In certain embodiments, the genetic locus comprising a GmKIX gene comprising a genotype associated with a large-seed phenotype has been gene edited. In certain embodiments, the genetic locus comprising the GmKIX8-1 gene Glyma.17G112800I comprising a genotype associated with a large-seed phenotype has been gene edited, such as for example, as described elsewhere herein.

Soybean Plants Comprising a Genomic Locus Associated with a Large-Seed Phenotype

Provided herein are soybean plants comprising a genomic locus of this disclosure associated with a large-seed phenotype. In certain embodiments, the soybean plant is a naturally occurring soybean variety comprising a genomic locus associated with a large-seed phenotype, such as can be identified by the methods disclosed herein. In certain embodiments, the soybean plant is made, such as by introgressing a large-seed phenotype genetic locus into a germplasm that does not comprises a large-seed phenotype genetic locus or by genetic editing as disclosed elsewhere herein.

In certain embodiments, the soybean plant is made by a method for producing a soybean plant comprising an introgressed genetic locus comprising a genotype associated with a large-seed phenotype of this disclosure. In certain embodiments, the genetic locus comprising a GmKIX gene comprising a genotype associated with a large-seed phenotype has been gene edited. In certain embodiments, the genetic locus comprising the GmKIX8-1 gene Glyma.17G112800I comprising a genotype associated with a large-seed phenotype has been gene edited, such as for example, as described elsewhere herein.

In certain embodiments, a soybean plant comprises an introgressed genetic locus comprising a genotype associated with a large-seed phenotype in a genomic region comprising a soybean GmKIX gene, wherein at least one marker linked to the introgressed large-seed phenotype genetic locus found in said soybean plant is characteristic of germplasm comprising a non-large-seed genetic locus but is not associated with germplasm comprising the large-seed phenotype genetic locus. In certain embodiments, a soybean plant comprises an introgressed genetic locus comprising a genotype associated with a large-seed phenotype in a genomic region comprising the soybean GmKIX8-1 gene Glyma.17G112800I, wherein at least one marker linked to the introgressed large-seed phenotype genetic locus found in said soybean plant is characteristic of germplasm comprising a non-large-seed genetic locus but is not associated with germplasm comprising the large-seed phenotype genetic locus. In certain embodiments, the soybean plant or a progeny thereof exhibits a large-seed phenotype.

In certain embodiments, the genetic locus comprising a genotype associated with the large-seed phenotype is a genomic region as described in detail above and disclosed elsewhere herein. In certain embodiments, the genotype associated with the large-seed phenotype is a genotype as described in detail above and disclosed elsewhere herein. In certain embodiments, genotyping is done with a SSR marker primer pair disclosed elsewhere herein.

### Molecular Assisted Breeding Techniques

Genetic markers that can be used in the practice of the instant disclosure include, but are not limited to, are Restriction Fragment Length Polymorphisms (RFLP), Amplified Fragment Length Polymorphisms (AFLP), Simple Sequence Repeats (SSR), Single Nucleotide Polymorphisms (SNP), Insertion/Deletion Polymorphisms (Indels), Variable Number Tandem Repeats (VNTR), and Random Amplified Polymorphic DNA (RAPD), and others known to those skilled in the art. Marker discovery and development in crops provides the initial framework for applications to marker-assisted breeding activities (US Patent Applications 2005/0204780, 2005/0216545, 2005/0218305, and 2006/00504538). The resulting “genetic map” is the representation of the relative position of characterized loci (DNA markers or any other locus for which alleles can be identified) along the chromosomes. The measure of distance on this map is relative to the frequency of crossover events between sister chromatids at meiosis.

As a set, polymorphic markers serve as a useful tool for fingerprinting plants to inform the degree of identity of lines or varieties. These markers form the basis for determining associations with phenotype and can be used to drive genetic gain. The implementation of marker-assisted selection is dependent on the ability to detect underlying genetic differences between individuals.

Certain genetic markers for use in the present disclosure include “dominant” or “codominant” markers. “Codominant markers” reveal the presence of two or more alleles (two per diploid individual). “Dominant markers” reveal the presence of only a single allele. The presence of the dominant marker phenotype (e.g., a band of DNA) is an indication that one allele is present in either the homozygous or heterozygous condition. The absence of the dominant marker phenotype (e.g., absence of a DNA band) is merely evidence that “some other” undefined allele is present. In the case of populations where individuals are predominantly homozygous and loci are predominantly dimorphic, dominant and codominant markers can be equally valuable. As populations become more heterozygous and multiallelic, codominant markers often become more informative of the genotype than dominant markers.

In another embodiment, markers that include. but are not limited, to single sequence repeat markers (SSR), AFLP markers, RFLP markers, RAPD markers, phenotypic markers, isozyme markers, single nucleotide polymorphisms (SNPs), insertions or deletions (Indels), single feature polymorphisms (SFPs, for example, as described in Borevitz et al. 2003 Gen. Res. 13:513-523), microarray transcription profiles, DNA-derived sequences, and RNA-derived sequences that are genetically linked to or correlated with large-seed phenotype loci, regions flanking large-seed phenotype loci, regions linked to large-seed phenotype loci, and/or regions that are unlinked to large-seed phenotype loci can be used in certain embodiments of the instant disclosure.

In one embodiment, nucleic acid-based analyses for determining the presence or absence of the genetic polymorphism (i.e. for genotyping) can be used for the selection of seeds in a breeding population. A wide variety of genetic markers for the analysis of genetic polymorphisms are available and known to those of skill in the art. The analysis may be used to select for genes, portions of genes, QTL, alleles, or genomic regions (genotypes) that comprise or are linked to a genetic marker that is linked to or correlated with large-seed phenotype loci, regions flanking large-seed phenotype loci, regions linked to large-seed phenotype loci, and/or regions that are unlinked to large-seed phenotype loci can be used in certain embodiments of the instant disclosure.

Nucleic acid analysis methods (e.g., genotyping) provided herein include, but are not limited to, PCR-based detection methods (for example, TaqMan assays), microarray methods, mass spectrometry-based methods and/or nucleic acid sequencing methods. In one embodiment, the detection of polymorphic sites in a sample of DNA, RNA, or cDNA may be facilitated through the use of nucleic acid amplification methods. Such methods specifically increase the concentration of polynucleotides that span the polymorphic site, or include that site and sequences located either distal or proximal to it. Such amplified molecules can be readily detected by gel electrophoresis, fluorescence detection methods, or other means.

A method of achieving such amplification employs the polymerase chain reaction (PCR) (Mullis et al. 1986 Cold Spring Harbor Symp. Quant. Biol. 51:263-273; European Patent 50,424; European Patent 84,796; European Patent 258,017; European Patent 237,362; European Patent 201,184; U.S. Pat. Nos. 4,683,202; 4,582,788; and 4,683,194), using primer pairs that are capable of hybridizing to the proximal sequences that define a polymorphism in its double-stranded form.

Methods for typing DNA based on mass spectrometry can also be used. Such methods are disclosed in U.S. Pat. Nos. 6,613,509 and 6,503,710, and references found therein.

Polymorphisms in DNA sequences can be detected or typed by a variety of effective methods well known in the art including, but not limited to, those disclosed in U.S. Pat. Nos. 5,468,613, 5,217,863; 5,210,015; 5,876,930; 6,030,787; 6,004,744; 6,013,431; 5,595,890; 5,762,876; 5,945,283; 5,468,613; 6,090,558; 5,800,944; 5,616,464; 7,312,039; 7,238,476; 7,297,485; 7,282,355; 7,270,981 and 7,250,252 all of which are incorporated herein by reference in their entireties. However, the compositions and methods of the present disclosure can be used in conjunction with any polymorphism typing method to type polymorphisms in genomic DNA samples. These genomic DNA samples used include but are not limited to genomic DNA isolated directly from a plant, cloned genomic DNA, or amplified genomic DNA.

For instance, polymorphisms in DNA sequences can be detected by hybridization to allele-specific oligonucleotide (ASO) probes as disclosed in U.S. Pat. Nos. 5,468,613 and 5,217,863. U.S. Pat. No. 5,468,613 discloses allele specific oligonucleotide hybridizations where single or multiple nucleotide variations in nucleic acid sequence can be detected in nucleic acids by a process in which the sequence containing the nucleotide variation is amplified, spotted on a membrane and treated with a labeled sequence-specific oligonucleotide probe.

Target nucleic acid sequence can also be detected by probe ligation methods as disclosed in U.S. Pat. No. 5,800,944 where sequence of interest is amplified and hybridized to probes followed by ligation to detect a labeled part of the probe.

Microarrays can also be used for polymorphism detection, wherein oligonucleotide probe sets are assembled in an overlapping fashion to represent a single sequence such that a difference in the target sequence at one point would result in partial probe hybridization (Borevitz et al., Genome Res. 13:513-523 (2003); Cui et al., Bioinformatics 21:3852-3858 (2005). On any one microarray, it is expected there will be a plurality of target sequences, which may represent genes and/or noncoding regions wherein each target sequence is represented by a series of overlapping oligonucleotides, rather than by a single probe. This platform provides for high throughput screening a plurality of polymorphisms. A single-feature polymorphism (SFP) is a polymorphism detected by a single probe in an oligonucleotide array, wherein a feature is a probe in the array. Typing of target sequences by microarray-based methods is disclosed in U.S. Pat. Nos. 6,799,122; 6,913,879; and 6,996,476.

Target nucleic acid sequence can also be detected by probe linking methods as disclosed in U.S. Pat. No. 5,616,464, employing at least one pair of probes having sequences homologous to adjacent portions of the target nucleic acid sequence and having side chains which non-covalently bind to form a stem upon base pairing of the probes to the target nucleic acid sequence. At least one of the side chains has a photoactivatable group which can form a covalent cross-link with the other side chain member of the stem.

Other methods for detecting SNPs and Indels include single base extension (SBE) methods. Examples of SBE methods include, but are not limited, to those disclosed in U.S. Pat. Nos. 6,004,744; 6,013,431; 5,595,890; 5,762,876; and 5,945,283. SBE methods are based on extension of a nucleotide primer that is adjacent to a polymorphism to incorporate a detectable nucleotide residue upon extension of the primer. In certain embodiments, the SBE method uses three synthetic oligonucleotides. Two of the oligonucleotides serve as PCR primers and are complementary to sequence of the locus of genomic DNA which flanks a region containing the polymorphism to be assayed. Following amplification of the region of the genome containing the polymorphism, the PCR product is mixed with the third oligonucleotide (called an extension primer) which is designed to hybridize to the amplified DNA adjacent to the polymorphism in the presence of DNA polymerase and two differentially labeled dideoxynucleosidetriphosphates. If the polymorphism is present on the template, one of the labeled dideoxynucleosidetriphosphates can be added to the primer in a single base chain extension. The allele present is then inferred by determining which of the two differential labels was added to the extension primer. Homozygous samples will result in only one of the two labeled bases being incorporated and thus only one of the two labels will be detected. Heterozygous samples have both alleles present, and will thus direct incorporation of both labels (into different molecules of the extension primer) and thus both labels will be detected.

In another method for detecting polymorphisms, SNPs and Indels can be detected by methods disclosed in U.S. Pat. Nos. 5,210,015; 5,876,930; and 6,030,787 in which an oligonucleotide probe having a 5′ fluorescent reporter dye and a 3′ quencher dye covalently linked to the 5′ and 3′ ends of the probe. When the probe is intact, the proximity of the reporter dye to the quencher dye results in the suppression of the reporter dye fluorescence, e.g. by Forster-type energy transfer. During PCR forward and reverse primers hybridize to a specific sequence of the target DNA flanking a polymorphism while the hybridization probe hybridizes to polymorphism-containing sequence within the amplified PCR product. In the subsequent PCR cycle DNA polymerase with 5′->3′ exonuclease activity cleaves the probe and separates the reporter dye from the quencher dye resulting in increased fluorescence of the reporter.

In another embodiment, the locus or loci of interest can be directly sequenced using nucleic acid sequencing technologies. Methods for nucleic acid sequencing are known in the art and include technologies provided by 454 Life Sciences (Branford, Conn.), Agencourt Bioscience (Beverly, Mass.), Applied Biosystems (Foster City, Calif.), LI-COR Biosciences (Lincoln, Nebr.), NimbleGen Systems (Madison, Wis.), Illumina (San Diego, Calif.), and VisiGen Biotechnologies (Houston, Tex.). Such nucleic acid sequencing technologies comprise formats such as parallel bead arrays, sequencing by ligation, capillary electrophoresis, electronic microchips, “biochips,” microarrays, parallel microchips, and single-molecule arrays, as reviewed by R.F. Service Science 2006 311:1544-1546.

The markers to be used in the methods of the present disclosure should preferably be diagnostic of origin in order for inferences to be made about subsequent populations. Experience to date suggests that SNP markers may be ideal for mapping because the likelihood that a particular SNP allele is derived from independent origins in the extant populations of a particular species is very low. As such, SNP markers appear to be useful for tracking and assisting introgression of QTLs, particularly in the case of genotypes.

### Gene Editing

Provided for herein are methods for producing a plant having a variant GmKIX gene that confers a large-seed phenotype to soybean plants. For example, provided for herein are methods for producing a plant having a variant GmKIX8-1 gene (Glyma.17G112800) that confers a large-seed phenotype to soybean plants. In certain embodiments, the variant is a reduced expression, downregulated, loss-of-function, and/or the like gene variant. Thus, it is understood that for purposes of this disclosure, a variant GmKIX gene, e.g., a variant GmKIX8-1 gene, of this disclosure is a genotype associated with a large-seed phenotype. Methods include, but are not limited to, gene editing tools such as CRISPR/Cas endonuclease-mediated editing, meganuclease-mediated editing, engineered zinc finger endonuclease-mediated editing, and traditional mutagenesis. For examples, certain embodiments comprise a precise gene editing in plant cells, callus, and/or germplasm explants using CRISPR/Cas system mediated by homology direct repair (HDR). In certain embodiments, the modifications can confer a large-seed phenotype to plants which are regenerated and selected using an in vitro culture approach.

In certain embodiments, a genetically-edited soybean plant comprising a GmKIX gene variant and/or protein can be obtained by using techniques that provide for genome editing in the plant. In certain embodiments, a plant comprising an endogenous GmKIX gene can be subjected to a genome editing technique wherein at least one nucleotide insertion, deletion, and/or substitution, in comparison to the corresponding unedited wild-type polynucleotide sequence is introduced, resulting in a large-seed phenotype.

The at least one nucleotide insertion, deletion, and/or substitution can be made anywhere in the gene including, for example, in the promoter region, the exons, the introns, and the untranslated regions (5′ and 3′ UTRs). The insertion, deletion, and/or substitution can be minimal, for example, a one nucleotide insertion, deletion, or substitution, or it can be more extensive, e.g., a deletion or insertion of up to 2, 3, 4, 5, 10, 15, 20, 25, 50, or more nucleotides such as between any of about 1, 2, 3, 4, 5, 10, 15, 20, 25, or 50 and any of about 2, 3, 4, 5, 10, 15, 20, 25, 50 or 100 nucleotides, or multiple nucleotide substitutions (e.g, 2, 3, 4, 5, 6, 7, 8, 9, 10, 25, 50, or 100, or any integer in between). Examples of methods for plant genome editing with clustered regularly interspaced short palindromic repeats (CRISPR)-associated (Cas)-polynucleotide modification template technology and a Cas endonuclease are at least disclosed by Bortesi and Fisher et al., 2015; Svitashev et al., 2015; Kumar and Jain, 2015; and in US Patent Appl. Pub. Nos. 20150082478, 20150059010, 20190352655, and 2020157554, which are specifically incorporated herein by reference in their entireties. Examples of methods involving cytosine base editors and adenine base editors are at least disclosed by Kim, Nature Plants, 2018 March, 4(3):148-151; Komor et al. (2016) Nature, 533:420-424, Komor et al., Sci Adv. 2017 August; 3(8):eaao4774; and Gaudelli et al., (2017) Nature 551(7681):464-471 and in US Patent Appl. Pub. Nos. 20180362590 and 20180312828, which are specifically incorporated herein by reference in their entireties.

Gene editing molecules for inducing a genetic modification in the plant cell or plant protoplast of the systems, methods, and compositions provided herein include, but are not limited to: (i) a polynucleotide selected from the group consisting of an RNA guide for an RNA-guided nuclease, a DNA encoding an RNA guide for an RNA-guided nuclease; (ii) a nuclease selected from the group consisting of an RNA-guided nuclease, an RNA-guided DNA endonuclease, a type II Cas nuclease, a Cas9, a type V Cas nuclease, a Cpfl, a CasY, a CasX, a C2cl, a C2c3, an engineered nuclease, a codon-optimized nuclease, a zinc-finger nuclease (ZFN), a transcription activator-like effector nuclease (TAL-effector nuclease), Argonaute, a meganuclease or engineered meganuclease; (iii) a polynucleotide encoding one or more nucleases capable of effecting site-specific modification of a target nucleotide sequence; and/or (iv) a donor template polynucleotide. In certain embodiments, at least one delivery agent is selected from the group consisting of solvents, fluorocarbons, glycols or polyols, surfactants; primary, secondary, or tertiary amines and quaternary ammonium salts; organosilicone surfactants; lipids, lipoproteins, lipopolysaccharides; acids, bases, caustic agents; peptides, proteins, or enzymes; cell-penetrating peptides; RNase inhibitors; cationic branched or linear polymers; dendrimers; counter-ions, amines or polyamines, osmolytes, buffers, and salts; polynucleotides; transfection agents; antibiotics; chelating agents such as ammonium oxalate, EDTA, EGTA, or cyclohexane diamine tetraacetate, non-specific DNA double-strand-break-inducing agents; and antioxidants; particles or nanoparticles, magnetic particles or nanoparticles, abrasive or scarifying agents, needles or microneedles, matrices, and grids.

Thus, provided for herein is an edited soybean GmKIX gene. For example, provided for herein is an edited soybean GmKIX8-1 gene (Glyma.17G112800). The edited gene can comprise an edit (e.g., at least one nucleotide insertion, deletion, and/or substitution) in any sequence/region of the gene, for example, in the promoter sequence, 5′ untranslated region (5′ UTR), exons, introns, 3′ UTR, etc., as long as the edit alters the expression and/or a characteristic of a gene product and/or activity of the encoded GmKIX protein. The at least one nucleotide insertion, deletion, and/or substitution can be short, e.g., one, two, or three nucleotides, or it can be longer, e.g., comprising 10, 20, 30, 40, 50, 75, or more nucleotides that have been inserted, deleted, and/or substituted. In certain embodiments, the GmKIX gene exhibits a loss-of-function, for example, is disrupted or knocked-out. For example, a polynucleotide variant of a promoter or untranslated region could cause a decrease or complete loss in expression of the GmKIX protein and thus an overall decrease in GmKIX activity in a cell, even if the GmKIX protein itself or its characteristics are unaltered. An edit in a protein coding region (e.g., exons) can result in a variant GmKIX protein amino acid sequence. The polynucleotide variant can include a frameshift, missense, and/or nonsense mutation. Such polynucleotide variant can result in a protein variant with at least one amino acid insertion, deletion, substitution, and/or truncation of the protein. The protein variant can have a conservative or a non-conservative substitution. In certain embodiments, the edit alters the activity of the variant GmKIX protein. In certain embodiments, the activity of GmKIX protein is reduced or abolished in comparison to the wild-type protein. In certain embodiments, when present in a plant and/or plant chromosome, the variant polynucleotide results a large-seed phenotype.

Certain embodiments provide for an edited soybean GmKIX gene comprising (i) a variant polynucleotide comprising a loss-of-function GmKIX gene variant, wherein the variant polynucleotide comprises at least one nucleotide insertion, deletion, and/or substitution in comparison to the corresponding unedited wild-type polynucleotide sequence, and wherein the variant polynucleotide exhibits reduced or loss of expression of the GmKIX gene in comparison to the wild-type polynucleotide sequence. Certain embodiments provide for an edited soybean GmKIX gene comprising (i) a variant polynucleotide comprising a loss-of-function GmKIX gene variant, wherein the variant polynucleotide comprises at least one nucleotide insertion, deletion, and/or substitution in comparison to the corresponding unedited wild-type polynucleotide sequence, and wherein the variant polynucleotide encodes a GmKIX protein variant having reduced activity in comparison to wild-type GmKIX protein.

Certain embodiments provide for an edited soybean GmKIX gene comprising (ii) a variant polynucleotide encoding a loss-of-function GmKIX protein variant or fragment thereof, wherein the variant polynucleotide comprises at least one nucleotide insertion, deletion, and/or substitution in comparison to the corresponding unedited wild-type polynucleotide sequence and does not encode for the wild-type GmKIX protein, and wherein the variant polynucleotide encodes for a GmKIX8-1 protein variant having reduced activity in comparison to wild-type GmKIX protein. In certain embodiments, the variant polynucleotide is operably linked to a polynucleotide comprising a promoter.

Certain embodiments provide for an edited soybean GmKIX gene comprising (iii) a variant polynucleotide comprising a GmKIX8-1 gene 3′ UTR, wherein the variant polynucleotide comprises at least one nucleotide insertion, deletion, and/or substitution in the 3′ UTR in comparison to the corresponding unedited wild-type polynucleotide sequence, and wherein the variant 3′ UTR results in reduced or loss of expression of the GmKIX gene in comparison to the wild-type polynucleotide sequence. In certain embodiments, the variant polynucleotide is operably linked to a polynucleotide comprising a promoter and/or a GmKIX protein coding region.

Certain embodiments provide for an edited soybean GmKIX gene comprising (iv) a variant polynucleotide comprising a GmKIX gene 5′ UTR, wherein the variant polynucleotide comprises at least one nucleotide insertion, deletion, and/or substitution in the 5′ UTR in comparison to the corresponding unedited wild-type polynucleotide sequence, and wherein the variant 5′ UTR results in reduced or loss of expression of the GmKIX gene in comparison to the wild-type polynucleotide sequence. In certain embodiments, the variant polynucleotide is operably linked to a polynucleotide comprising a promoter and/or a GmKIX protein coding region.

Certain embodiments provide for an edited soybean GmKIX gene comprising (v) a variant polynucleotide comprising a GmKIX gene promoter, wherein the variant polynucleotide comprises at least one nucleotide insertion, deletion, and/or substitution in the promoter in comparison to the corresponding unedited wild-type polynucleotide sequence, and wherein the variant promoter results in reduced or loss of expression of the GmKIX gene in comparison to the wild-type polynucleotide sequence. In certain embodiments, the variant polynucleotide is operably linked to a polynucleotide comprising a GmKIX protein coding region.

Certain embodiments provide for an edited soybean GmKIX gene comprising (vi) a variant polynucleotide comprising a GmKIX gene intron, wherein the variant polynucleotide comprises at least one nucleotide insertion, deletion, and/or substitution in the intron in comparison to the corresponding unedited wild-type polynucleotide sequence, and wherein the variant intron results in reduced or loss of expression of the GmKIX gene in comparison to the wild-type polynucleotide sequence. In certain embodiments, the variant polynucleotide is operably linked to a polynucleotide comprising at least one GmKIX gene exon.

Certain embodiments provide for an edited soybean GmKIX gene comprising (vii) a variant polynucleotide comprising a GmKIX gene exon, wherein the variant polypeptide comprises at least one nucleotide insertion, deletion, and/or substitution in the exon in comparison to the corresponding unedited wild-type polynucleotide sequence, and wherein the variant exon results in reduced or loss of expression of the GmKIX gene in comparison to the wild-type polynucleotide sequence. In certain embodiments, the variant polynucleotide is operably linked to a polynucleotide comprising a promoter and/or a GmKIX gene intron. Certain embodiments provide for an edited soybean GmKIX gene comprising (vii) a variant polynucleotide comprising a GmKIX gene exon, wherein the variant polypeptide comprises at least one nucleotide insertion, deletion, and/or substitution in the exon in comparison to the corresponding unedited wild-type polynucleotide sequence, and wherein the variant exon encodes a GmKIX8 protein variant having reduced activity in comparison to wild-type GmKIX protein. In certain embodiments, the variant polynucleotide is operably linked to a polynucleotide comprising a promoter and/or a GmKIX gene intron.

In certain embodiments, the edited soybean GmKIX gene comprises a variant polypeptide comprising a loss-of-function GmKIX gene variant. For example, comprising at least one nucleotide deletion in the promoter in comparison to the corresponding unedited wild-type polynucleotide sequence, wherein the nucleotide deletion results in reduced or loss of expression of the GmKIX gene in comparison to the corresponding wild-type polynucleotide sequence. In certain embodiments, the variant polynucleotide comprises at least one nucleotide insertion, deletion, and/or substitution in comparison to the corresponding unedited wild-type polynucleotide sequence and does not encode for the wild-type GmKIX protein, wherein the variant polynucleotide encodes for a GmKIX protein variant having reduced activity in comparison to wild-type GmKIX protein. In certain embodiments, the nucleotide insertion, deletion, and/or substitution results in a frameshift mutation and/or a nonsense mutation.

Certain embodiments provide for an edited soybean GmKIX8-1 gene comprising (i) a variant polynucleotide comprising a loss-of-function GmKIX8-1 gene variant, wherein the variant polynucleotide comprises at least one nucleotide insertion, deletion, and/or substitution in comparison to the corresponding unedited wild-type polynucleotide sequence, and wherein the variant polynucleotide exhibits reduced or loss of expression of the GmKIX8-1 gene in comparison to the wild-type polynucleotide sequence. Certain embodiments provide for an edited soybean GmKIX8-1 gene comprising (i) a variant polynucleotide comprising a loss-of-function GmKIX8-1 gene variant, wherein the variant polynucleotide comprises at least one nucleotide insertion, deletion, and/or substitution in comparison to the corresponding unedited wild-type polynucleotide sequence, and wherein the variant polynucleotide encodes a GmKIX8-1 protein variant having reduced activity in comparison to wild-type GmKIX8-1 protein.

Certain embodiments provide for an edited soybean GmKIX8-1 gene comprising (ii) a variant polynucleotide encoding a loss-of-function GmKIX8-1 protein variant or fragment thereof, wherein the variant polynucleotide comprises at least one nucleotide insertion, deletion, and/or substitution in comparison to the corresponding unedited wild-type polynucleotide sequence and does not encode for the wild-type GmKIX8-1 protein, and wherein the variant polynucleotide encodes for a GmKIX8-1 protein variant having reduced activity in comparison to wild-type GmKIX8-1 protein. In certain embodiments, the variant polynucleotide is operably linked to a polynucleotide comprising a promoter.

Certain embodiments provide for an edited soybean GmKIX8-1 gene comprising (iii) a variant polynucleotide comprising a GmKIX8-1 gene 3′ UTR, wherein the variant polynucleotide comprises at least one nucleotide insertion, deletion, and/or substitution in the 3′ UTR in comparison to the corresponding unedited wild-type polynucleotide sequence, and wherein the variant 3′ UTR results in reduced or loss of expression of the GmKIX8-1 gene in comparison to the wild-type polynucleotide sequence. In certain embodiments, the variant polynucleotide is operably linked to a polynucleotide comprising a promoter and/or a GmKIX8-1 protein coding region.

Certain embodiments provide for an edited soybean GmKIX8-1 gene comprising (iv) a variant polypeptide comprising a GmKIX8-1 gene 5′ UTR, wherein the variant polynucleotide comprises at least one nucleotide insertion, deletion, and/or substitution in the 5′ UTR in comparison to the corresponding unedited wild-type polynucleotide sequence, and wherein the variant 5′ UTR results in reduced or loss of expression of the GmKIX8-1 gene in comparison to the wild-type polynucleotide sequence. In certain embodiments, the variant polynucleotide is operably linked to a polynucleotide comprising a promoter and/or a GmKIX8-1 protein coding region.

Certain embodiments provide for an edited soybean GmKIX8-1 gene comprising (v) a variant polynucleotide comprising a GmKIX8-1 gene promoter, wherein the variant polynucleotide comprises at least one nucleotide insertion, deletion, and/or substitution in the promoter in comparison to the corresponding unedited wild-type polynucleotide sequence, and wherein the variant promoter results in reduced or loss of expression of the GmKIX8-1 gene in comparison to the wild-type polynucleotide sequence. In certain embodiments, the variant polynucleotide is operably linked to a polynucleotide comprising a GmKIX8-1 protein coding region.

Certain embodiments provide for an edited soybean GmKIX8-1 gene comprising (vi) a variant polynucleotide comprising a GmKIX8-1 gene intron, wherein the variant polynucleotide comprises at least one nucleotide insertion, deletion, and/or substitution in the intron in comparison to the corresponding unedited wild-type polynucleotide sequence, and wherein the variant intron results in reduced or loss of expression of the GmKIX8-1 gene in comparison to the wild-type polynucleotide sequence. In certain embodiments, the variant polynucleotide is operably linked to a polynucleotide comprising at least one GmKIX8-1 gene exon.

Certain embodiments provide for an edited soybean GmKIX8-1 gene comprising (vii) a variant polynucleotide comprising a GmKIX8-1 gene exon, wherein the variant polypeptide comprises at least one nucleotide insertion, deletion, and/or substitution in the exon in comparison to the corresponding unedited wild-type polynucleotide sequence, and wherein the variant exon results in reduced or loss of expression of the GmKIX8-1 gene in comparison to the wild-type polynucleotide sequence. Certain embodiments provide for an edited soybean GmKIX8-1 gene comprising (vii) a variant polynucleotide comprising a GmKIX8-1 gene exon, wherein the variant polypeptide comprises at least one nucleotide insertion, deletion, and/or substitution in the exon in comparison to the corresponding unedited wild-type polynucleotide sequence, and wherein the variant exon encodes a GmKIX8-1 protein variant having reduced activity in comparison to wild-type GmKIX8-1 protein. In certain embodiments, the variant polynucleotide is operably linked to a polynucleotide comprising a promoter and/or a GmKIX8-1 gene intron. In certain embodiments, the variant polypeptide comprises a deletion of at least a portion of exon 1, exon 2, or both exon 1 and exon 2 of the GmKIX8-1 gene.

In certain embodiments the edited soybean GmKIX8-1 gene comprises a variant polypeptide comprising a loss-of-function GmKIX8-1 gene variant. In certain embodiments, the variant polynucleotide comprises a deletion of a portion of the 3′ end of exon 1 of the GmKIX8-1 gene, a deletion of the intron between exon 1 and exon 2 of the GmKIX8-1 gene, and a deletion of a portion the 5′ end of exon 2 of the GmKIX8-1 gene in comparison to the corresponding unedited wild-type polynucleotide sequence. In certain embodiments, the variant polynucleotide comprises at least one nucleotide deletion in the promoter in comparison to the corresponding unedited wild-type polynucleotide sequence, wherein the nucleotide deletion results in reduced or loss of expression of the GmKIX8-1 gene in comparison to the corresponding wild-type polynucleotide sequence. In certain embodiments the variant polynucleotide encoding a loss-of-function GmKIX8-1 protein variant or fragment thereof, wherein the variant polynucleotide comprises at least one nucleotide insertion, deletion, and/or substitution in comparison to the corresponding unedited wild-type polynucleotide sequence and does not encode for the wild-type GmKIX8-1 protein, and wherein the variant polynucleotide encodes for a GmKIX8-1 protein variant having reduced activity in comparison to wild-type GmKIX8-1 protein. In certain embodiments, the nucleotide insertion, deletion, and/or substitution results in a frameshift mutation and/or a nonsense mutation.

In certain of any embodiments of an edited soybean GmKIX gene of this disclosure, the variant polynucleotide is integrated into the genome, for example the nuclear genome, of a cell. In certain embodiments, the genome is of a plant cell. Thus, the disclosure provides for example for a plant nuclear genome comprising an edited GmKIX gene of this disclosure. In certain embodiments, the variant polynucleotide is heterologous to the genome. In certain embodiments, the variant polynucleotide is operably linked to an endogenous promoter of the genome, for example, a wild-type GmKIX gene promoter. In certain embodiments, the edited gene or the genome further comprises a wild-type or variant polynucleotide encoding (a) a transit peptide, a vacuolar targeting peptide, and/or an endoplasmic reticulum targeting peptide; (b) a plastid targeting peptide; and/or (c) a polyadenylation or transcriptional termination signal, wherein the polynucleotides of (a), (b), and/or (c) are operably linked to the polypeptide encoding the GmKIX protein.

Also provided for herein is a cell comprising the edited gene or genome of this disclosure. In certain embodiments, the genome is a nuclear genome. In certain embodiments, the cell is a plant, yeast, mammalian, or bacterial cell. In certain embodiments, cell is a plant cell. In certain embodiments, the cell is a plant cell that is non-regenerable.

Also provided for herein is a soybean plant comprising the edited soybean GmKIX gene (e.g., GmKIX8-1 gene) or genome of this disclosure. In certain embodiments, the soybean plant is a gene-edited soybean plant comprising a variant polynucleotide that comprises a targeted loss-of-function GmKIX gene variant which comprises an insertion, substitution, and/or a deletion in a GmKIX gene that reduces expression of the GmKIX gene compared to wild-type expression and/or encodes a GmKIX protein variant having reduced activity in comparison to wild-type GmKIX protein. For example, a gene-edited soybean plant comprising a variant polynucleotide that comprises a targeted loss-of-function GmKIX gene variant which comprises an insertion, substitution, and/or a deletion in the GmKIX8-1 gene Glyma.17G112800 that reduces expression of the GmKIX8-1 gene Glyma.17G112800 compared to wild-type expression and/or encodes a GmKIX8-1 protein variant having reduced activity in comparison to wild-type GmKIX8-1 protein such as described in detail elsewhere herein.

In certain embodiments, the edited soybean GmKIX gene (e.g., GmKIX8-1) or genome of this disclosure confers to the plant larger seed size in comparison to a control plant that lacks the edited gene genome. In certain embodiments, the larger seed size in comparison to a control plant that lacks the edited gene or genome is at least about 3%, 5%, 10%, 15%, 20%, 25%, 30%, 35%, 40%, or 50% higher 100-seed weight. In certain embodiments, the edited soybean GmKIX gene or genome confers to the plant a large-seed phenotype.

Certain embodiments provide for a plant part of the soybean plant of this disclosure, wherein the plant part comprises the edited soybean GmKIX gene (e.g., GmKIX8-1) or genome of this disclosure. In certain the plant part is a seed, stem, leaf, root, or flower. In certain embodiments, the plant part is a soybean seed.

Also provided for herein is a method for producing a soybean plant comprising the edited soybean GmKIX gene (e.g., GmKIX8-1) or plant genome of this disclosure, wherein edited soybean GmKIX gene exhibits reduced or loss of expression of the GmKIX gene in comparison to the wild-type polynucleotide sequence. Also provided for herein is a method for producing a soybean plant comprising the edited soybean GmKIX gene (e.g., GmKIX8-1) or plant genome of this disclosure, wherein edited soybean GmKIX gene encodes a GmKIX protein variant having reduced activity in comparison to wild-type GmKIX protein. In certain embodiments, the soybean plant obtained exhibits a large-seed phenotype. In certain embodiments, the method comprises introducing into a plant cell one or more gene-editing molecules such as those disclosed above that target the endogenous soybean GmKIX gene to introduce at least one nucleotide insertion, deletion, and/or substitution into the endogenous GmKIX gene.

In certain embodiments, the method for producing a soybean plant comprising the edited soybean GmKIX gene (e.g., GmKIX8-1) or plant genome of this disclosure comprises the steps of: (i) providing to a plant cell, tissue, part, or whole plant an endonuclease or an endonuclease and at least one guide RNA, wherein the endonuclease or guide RNA and endonuclease can form a complex that can introduce a double-strand break at a target site in a genome of the plant cell, tissue, part, or whole plant; (ii) obtaining a plant cell, tissue, part, or whole plant wherein at least one nucleotide insertion, deletion, and/or substitution has been introduced into the corresponding wild-type polynucleotide sequence; and (iii) selecting a plant obtained from the plant cell, tissue, part or whole plant of step (ii) comprising the edited soybean GmKIX8-1 gene. In certain embodiments, the selected soybean plant exhibits a large-seed phenotype. In certain embodiments, the endonuclease is a Cas endonuclease. In certain embodiments, the guide RNA is a clustered regularly interspaced short palindromic repeats (CRISPR)-associated (Cas)-guide RNA. And, in certain embodiments, the guide RNA comprises GGCCTTACGAGTGCGTGAGA (gRNA1; SEQ ID NO: 87) and/or GCTCCCCGTGGTGGTTCTCA (gRNA2; SEQ ID NO: 88).

Also provided for herein is a seed produced by a soybean plant of this disclosure wherein said seed comprises a detectable amount of the genetic locus that comprises a genotype associated with a large-seed phenotype. In certain embodiments, the seed comprises an endogenous edited gene comprising the variant polynucleotide. In certain embodiments, the seed is coated with a composition comprising an insecticide and/or a fungicide. Also provided for herein is a plant propagation material comprising the coated seed.

Certain embodiments provide for a plant or seed of this disclosure for use in a method of plant breeding, crop production, or for making a processed soybean plant product.

This disclosure also provides for a method of producing/breeding a soybean plant with a large-seed phenotype. Such method comprises crossing a soybean plant comprising a genotype associated with a large-seed phenotype of this disclosure with one or more other plants to produce a population of progeny plants. In certain embodiments, the one or more of the other plants comprises an endogenous edited gene. In certain embodiments, the method further comprises screening the population of progeny plants to identify large-seed phenotype plants. In certain embodiments, the population of plants is screened by genotyping to detect a GmKIX8-1 gene variant polynucleotide. In certain embodiments, the method further comprises selecting a progeny plant based on its genotype and/or phenotype.

### Knock Down

One of ordinary skill in the art will understand that in addition to gene editing, other methods of accomplishing reduced expression of the GmKIX8-1 gene and/or its protein activity/function, such as knock down technologies, would also increase seed weight. Examples of such technology include antisense oligonucleotides, RNAi, and miRNA.

### Method of Producing a Commercial Seed Lot

To generate enough seed for commercial distribution, the seed of commercial crops can be gathered from a plurality of plants and pooled together to create a seed lot. A commercial seed lot of a crop preferably contains a plurality of seeds that share similar or identical characteristics such as species, variety, genetic makeup, and/or similar germination rates. Provided for herein is a method of producing a commercial crop seed lot of soybean seeds comprising in their genomes a large-seed phenotype genetic locus and/or genotype associated with a large-seed phenotype of this disclosure. In certain embodiments, the genetic locus is an introgressed locus as described herein. In certain embodiments, the genetic locus has been gene edited. In certain embodiments, the method comprises the steps of: (a) producing a population of soybean plants from a soybean plant comprising a genotype associated with a large-seed phenotype and at least one linked marker found in said second soybean plant comprising a non-large seed phenotype genetic locus but not found in said first soybean plant; and (b) harvesting a commercial seed lot, wherein the harvested crop seed lot comprises a plurality of seeds that comprise in their genomes at least one introgressed large-seed phenotype genetic locus.

In certain embodiments, the seed lot comprise at least 100 seeds, at least 500 seeds, at least 1,000 seeds, at least 5,000 seeds, at least 10,000 seeds, at least 25,000 seeds, at least 50,000 seeds, or at least 100,000 seeds.

In certain embodiments, the plurality of seeds that comprise in their genomes at least one introgressed genetic locus of this disclosure having a genotype associated with a large-seed phenotype constitute at least 85%, at least 90%, at least 91%, at least 92%, at least 93%, at least 94%, at least 95%, at least 96%, at least 97%, at least 98%, or at least 99% of the seed of the harvested seed lot. In certain embodiments, the plurality of seeds that comprise in their genomes at least one introgressed genetic locus of this disclosure having a genotype associated with a large-seed phenotype constitute 100% of the seed of the harvested seed lot.

### Packaging and Distribution

In certain embodiments, such as for a method for producing a commercial crop seed lot, the seed of the harvested commercial crop seed lot is packaged into one or more bags. Such packaging results in one or more packaged seed bags. In one embodiment, the packaged seeds, such as in packaged seed bags, are further distributed to growers for use in crop production.

One of skill in the art will recognize that packaged seed bags of a commercial crop seed lot destined for distribution to growers for use in crop production will preferably comprise a large number of seeds. For example, at least one hundred seeds, at least one thousand seeds, at least ten thousand seeds, at least one hundred thousand seeds, or at least one million seeds.

### Commercial Crop Seed Lot

Provided for herein is a commercial crop seed lot wherein a plurality of seeds comprise a large-seed phenotype genetic locus and/or genotype associated with a large-seed phenotype of this disclosure. In certain embodiments a commercial crop seed lot comprises a plurality of seeds comprising a large-seed phenotype genetic locus that has been introgressed. Commercial crop seed lots of soybean seeds can be made by the methods described in detail elsewhere herein.

For certain crop species, cross-pollination of certain distinct plant lines can result in hybrid offspring exhibiting a highly desirable heterosis or hybrid vigor which advantageously provides increased yields of the desired crop. In one embodiment, the crossing of parental plants produces a commercial crop seed lot that provides plants that yield at least 5% more than plants produced by selfing either the male parent plants or the female parent plants used to obtain the commercial crop seed lot when the crossed plants and the selfed plants are grown under the same field conditions. In a further embodiment, the crossing of parental plants produces a commercial crop seed lot that provides plants that yield at least 10% more than plants produced by selfing either the male parent plants or the female parent plants used to obtain the commercial crop seed lot when the crossed plants and the selfed plants are grown under the same field conditions. In yet a further embodiment, the crossing of parental plants produces a commercial crop seed lot that provides plants that yield at least 15% more than plants produced by selfing either the male parent plants or the female parent plants used to obtain the commercial crop seed lot when the crossed plants and the selfed plants are grown under the same field conditions.

One of skill in the art will recognize that certain standards may be set—as may be set for certification—for a commercial crop seed lot. These standards may vary according to the crop selected and different classes of standards may exist such as “breeder,” “foundation,” “registered,” and “certified.”

One of skill in the art will recognize that the preceding standards are illustrative and that standards may vary depending on geographical location and as set by different regulatory entities. The standards disclosed herein are consistent with standards that are practiced in the field of commercial seed certification. Such standards are useful guidelines, however, the present disclosure is not to be interpreted as limited only to such standards or crops, but also to encompass other standards and crops as are known to those skilled in the art.

### Method of Increasing Soybean Seed Weight

Certain embodiments of this disclosure are drawn to downregulating or reducing GmKIX8-1 expression and/or the expression of a homologous GmKIX gene such as GmKIX8-2, by use of any of the compositions or methods disclosed herein, such as gene editing, or otherwise know in the art. Certain embodiments are also drawn to reducing protein activity and/or molecular pathway interactions, as described in detail elsewhere herein, of proteins encoded by the GmKIX8-1 gene and/or a homologous GmKIX gene such as GmKIX8-2, by use of any of the compositions or methods disclosed herein, such as gene editing, or otherwise known in the art. For example, certain embodiments provide for a method of increasing soybean seed weight comprising reducing or abolishing expression of the GmKIX8-1 gene. For examples, certain embodiments provide for a method of increasing soybean seed weight comprising reducing or abolishing activity of the GmKIX8-1 protein. In certain embodiments the reduction in expression and/or activity is accomplished by gene editing technology as described in greater detail above. Thus, it is understood that in certain embodiments, a method for producing a soybean plant comprising an edited GmKIX gene (e.g., GmKIX8-1) or plant genome of this disclosure can also be a method of increasing soybean seed weight. In certain embodiments the reduction in expression and/or activity is accomplished by a gene knockdown technology, for example antisense oligonucleotides, RNAi, and miRNA.

### Examples

### Materials and Methods

### Plant Material, Phenotyping, Genetic Crosses and Growth Conditions

A fast neutron K83 mutant was identified in a soybean (Glycine max (L.) Merr) fast neutron mutant population developed at the University of Missouri using cultivar Williams 82 as genetic background (Stacey et al., 2016). Phenotypic screens for altered leaf and seed appearance (e.g. color, size, shape) were done on M3 seeds. Back-crosses were performed by pollinating emasculated flowers of the parental cultivar Williams 82 with pollen from mutant plants grown at the Bradford Research and Experiment Center (BREC), University of Missouri, Columbia. Growth and phenotypic observations of BClF2 and BClF3 plants were also performed for plants grown at the BREC fields in 2016 and 2017. Big seed soybean plant introductions (PIs) Tachinagaha PI561396, Keunolkong1 PI597483, and Keunolkong2 PI594021 were obtained from the USDA Soybean Germplasm (world wide web at ars-grin.gov) and planted at BREC in 2018.

### Measurements of Leaf Growth Parameters

For leaf area measurements, 15-20 seedlings were grown in soil (Pro-Mix soil Premier Horticulture) at 27° C. with a 16 h:8 h, light:dark photoperiod under glasshouse conditions. To determine cotyledon and leaf area, 2-week-old cotyledons and 4-week-old fully expanded leaves were scanned with a Perfection v500 Scanner (Epson, Long Beach, Calif., USA), and the area was measured using IMAGEJ (world wide web at rsb.info.nih.gov/ij). For epidermal cell size and number measurements, nail polish was painted on the abaxial surface of cotyledons and allowed to dry for 5 min. Nail polish copies prepared from the transparent nail polish imprints were then imaged using a DM 5500B Compound Microscope (Leica Microsystems Inc., Wetzlar, Germany). Abaxial epidermal cell and stomatal numbers were obtained from cotyledons. Epidermal cell size was measured using IMAGEJ. Stomatal index was calculated by dividing the stomata number by the total cell number.

### Comparative Genome Hybridization (CGH) and Data Analysis to Identify Copy Number Variations (CNVs)

Comparative genome hybridization was performed using a 696 139-feature soybean CGH microarray as described previously (Haun et al., 2011; Bolon et al., 2011; Stacey et al., 2016). The oligonucleotide probes are 50- to 70-mers in length, spaced at c. 1.1 kb intervals and were designed by Roche NimbleGen (Madison, Wis., USA) based on the sequenced Williams 82 genome. K83 and Williams 82 chromosomal DNA were isolated from young leaf tissues using the Plant DNeasy Mini Kit (Qia-gen) and labeled with cy3 and cy5, respectively. Reagents and materials for DNA labeling hybridizations and data acquisition were used according to the manufacturer's established guidelines. For each CGH dataset, the average and standard deviation (SD) values for corrected log₂ ratios of the 696 139 unique probes were obtained. Significant CNV events were termed additions or deletions following previously set criteria (Bolon et al., 2011).

Copy Number Variation Determination by Quantitative Polymerase Chain Reaction (qPCR)

Chromosomal DNA was isolated from young leaf tissue using the GeneJET Plant Genomic DNA Purification Kit (Thermo Scientific, Waltham, Mass., USA). Variations in copy number were determined using an ABI17500 real-time PCR following the SYBR Green method (Applied Biosystems, Waltham, Mass., USA), normalized to CNVref1 (SEQ ID NO: 27) and CNVref2 (SEQ ID NO: 28) and analyzed with QBASE PLUS software (Hellemans et al., 2007). RNA isolation, cDNA synthesis and transcript level analysis

RNA was extracted using TRIzol reagent (Invitrogen), purified with the RNeasy Plant Mini Kit (Qiagen) and treated with TURBO DNase following the manufacturer's instructions. cDNA was synthesized using oligo(dT) primers (15-mer) and Moloney Murine Leukemia Virus (M-MLV) reverse transcriptase enzyme (Promega) following the manufacturer's instructions. Transcript levels of genes were determined by quantitative real-time polymerase chain reaction (qRT-PCR) on leaves or shoot tips using an ABI17500 real-time PCR following the SYBR Green method (Applied Biosystems). Gene expression levels were normalized to the expression of the soybean housekeeping genes cons6 and cons4 (Libault et al., 2008) and analyzed with QBASE PLUS.

### Vector/Plasmid Construction and Agrobacterium Transformation

A dual CRISPR/Cas9 system was developed as previously described by Do et al. (2019). Briefly, soybean specific single-guide RNA sequences were designed using web tools (CCTop; Stemmer et al., 2015). Two gRNAs (gRNA1 and gRNA2) were used to create defined deletions c. 200 bp within the first and second exons of GmKIX8-1. For each gRNA, a pair of DNA oligonucleotides (Table 2) were synthesized by Integrated DNA Technologies, Inc. (IDT; Coralville, Iowa, USA) and annealed to generate dimers. Subsequently, the annealed DNA was cloned into BbsI sites of pAtU6-26-SK to create pSK-AtU6-26-gRNA, and sequence integrity was confirmed by Sanger sequencing. In order to construct a functional Cas9 expression vector for targeted mutagenesis, pSK-AtU6-26-gRNA1 was cut with BamHI-SpeI, pSK-AtU6-26-gRNA2 was cut with BamHI-EcoRI, and 35S-Cas9-SK was digested with HindIII-SpeI. These three fragments were assembled into pFGC5941 (GenBank accession no. AY310901) by HindIII-EcoRI restriction digestion followed by ligation to give the pFGC-KIX1-CRISPR construct. The positive plasmids were introduced into Agrobacterium tumefaciens strain AGL1 by electroporation and used for subsequent soybean stable transformations.

To construct the GmKIX8-1 GFP fusion vector, GmKIX8-1 CDS from Williams 82 and PI597483 were amplified by PCR using the cDNA library described above in “RNA Isolation, cDNA synthesis and transcript level analysis” as template. The amplified PCR product without a stop codon was cloned into pDONR221 and then sub-cloned into the pMDC83 vector with a green fluorescent protein (GFP) tag at the C-terminus (Curtis & Grossniklaus, 2003). The resulting plasmid construct, pMDC83-GmKIX8-1-GFP, was transformed into A. tumefaciens GV3101 by electroporation.

For dual luciferase (LUC) reporter assays, a 1.6 kb genomic fragment located upstream of the predicted translational site encoding the promoter region of GmKIX8-1 was amplified by PCR using genomic DNA isolated from Williams 82 or PI597483. The PCR product was cloned using the pGEMT Easy Vector Systems for sequencing. The inserts were released by NotI and HindIII digestion, and then subcloned into the transient expression vector pGreenII0800-LUC (Hellens et al., 2005) to generate pGmKIX-W82::LUC and pGmKIX-PI597483::LUC, encoding promoter sequences amplified from Williams 82 and PI597483, respectively. Serial deletion vectors, pDel1::LUC, pDel2::LUC and pDel3::LUC were constructed from pGEMT-pW82 using specific primers (Table 2) and subcloned into the transient vector as described above for pGmKIX-W82::LUC and pGmKIX-PI597483::LUC. The plasmids were transformed into A. tumefaciens strain GV3101 containing the pSoup helper plasmid.

### Plant Transformation and Microscopy

The soybean genotype ‘Maverick’ was transformed using the Agrobacterium-mediated cotyledon node system using established protocols (Zeng et al., 2004). Agrobacterium-mediated transient expression in Nicotiana benthamiana (tobacco) leaves was done as described in a study by Bashandy et al. (2015). Briefly, Agrobacterium (strain GV3101) harboring the GFP fusion plasmids was grown overnight with Luria Broth (LB) supplemented with rifampicin and kanamycin (100 μg ml⁻¹) in a shaking incubator. Agrobacterium containing pMDC83 was used as control. Bacterial cells were collected by centrifugation at 3200 g for 10 min at room temperature and resuspended in Mg-MES buffer (200 μM acetosyringone, 10 mM MgCl₂, 10 mM 2-(N-morpholino) ethane sulfonic acid (MES), pH 6.0), and adjusted to a final density of OD600=0.5. The cell suspensions were kept at room temperature for at least 3 h before infiltration into tobacco leaves. Three top leaves from 4-wk-old tobacco plants, grown at 24° C. under a 16 h:8 h, light:dark photoperiod, were used for infiltration. Agrobacterium suspension was infiltrated by applying pressure to the abaxial surface of the leaf with a 1 ml plastic syringe without a needle. After agroinfiltration, the plants were kept in the growth room for 2 d before observation. Tissue sections from infiltrated leaf areas were viewed under a LSM 510 META NLO two-photon-scanning confocal microscope (Zeiss) with a 940 water objective.

### Western-Blot Analysis

Total protein was extracted from infiltrated leaf areas of N. benthamiana using an extraction buffer containing 50 mM Tris (pH 7.5), 150 mM NaCl, 0.5% Triton-X 100, and 1 9 protease inhibitor. The solution was then centrifuged at 20 000 g for 15 min to remove the debris. Proteins were separated by electrophoresis with 12% sodium dodecyl sulfate-polyacrylamide gel electrophoresis (SDS-PAGE) gel and detected by immunoblotting with a polyclonal anti-GFP antibody (Invitrogen, A-11122, 1:1000). Ponceau S (Sigma) staining was performed as a loading control.

### Dual Luciferase Reporter Assays

Leaves from 4-week-old tobacco plants, grown at 24° C. under a 16 h:8 h, light:dark photoperiod, were infiltrated with Agrobacterium (strain GV3101) harboring the LUC fusion plasmids as described above in “Vector/plasmid construction and Agrobacterium transformation” section. Three days after infiltration, c. 5 mg of infiltrated leaf tissue was sampled and subjected to LUC quantification using the dual luciferase assay reagents (Promega) as described previously (Moyle et al., 2017). Four independent biological samples were assayed.

### Genotyping of Plants to Identify CRISPR/Cas9-Induced Mutations in GmKIX8-1

The DNA region spanning the two targets in GmKIX8-1 was PCR-amplified using flanking primer pairs and Phusion High-Fidelity DNA Polymerase (New England Biolabs, Ipswich, Mass., USA). The PCR products were separated by 1.5% agarose gel electrophoresis, and the DNA band of interest was purified from the gel and ligated to pGEMT Easy Vector Systems (Promega, Madison, Wis., USA) for sequencing. Sequencing was performed at the University of Missouri DNA Core Facility. The DNA sequences derived from the putative gmkix8-1 mutant and wild-type plants were aligned using the online program MUSCLE (world wide web at ebi.ac.uklTools/msa/muscle/) to identify the mutations induced by CRISPR/Cas9. Similar PCR-based genotyping and sequencing approaches were utilized to identify the inheritance of the GmKIX8-1 mutation at the T1 generation.

TABLE 2
Primers used in the study for PCR, qRT-PCR, and cloning.
q-RT PCR primers

| | |
|---|---|
| F-qPCR-PPD2-CHr20 | CATGCAGCTTGCGGTGAGTCCTG (SEQ ID NO: 1) |
| R-qPCR-PPD2-CHr20 | GCAATGACACTTTTCTGCTTGCC (SEQ ID NO: 2) |
| F-qPCR-PPD2-CHr10 | ATGTAGAGGGTCAGGAACACCG (SEQ ID NO: 3) |
| R-qPCR-PPD2-CHr10 | AGCTGATCTGAACCACTGGAAAC (SEQ ID NO: 4) |
| F-qPCR-KIX1 | TGAGACAACCGAGACAGGAGGC (SEQ ID NO: 5) |
| R-qPCR-KIX1 | CAACAGGTTTAGGAGGCAGACAAG (SEQ ID NO: 6) |
| F-qPCR-KIX2 | CTCCCTCTGTGTCTCATAAACC (SEQ ID NO: 7) |
| R-qPCR-KIX2 | GCTGGCGATGACAAGGGAGGC (SEQ ID NO: 8) |
| F-qPCR-GRF1-chr10 | CCACAACCATCTCCATTGGCTGG (SEQ ID NO: 9) |
| R-qPCR-GRF1-chr10 | GGCTGTTGGGAGTACAGGAGCG (SEQ ID NO: 10) |
| F-qPCR-GRF1-CHR20 | CAGATGCAGCCTATCATGGCAG (SEQ ID NO: 11) |
| R-qPCR-GRF1-CHR20 | ACCAGGCATTGGAGATGGTTGG (SEQ ID NO: 12) |
| F-qPCR-CYCD3-1chr20 | GCCATTCTCCAACTCTCAAATGTC (SEQ ID NO: 13) |
| R-qPCR-CYCD3-1chr20 | AGAGGCTCTGGTGGTGAATATAATG (SEQ ID NO: 14) |
| F-qPCR-CYCD3-1chr10 | GAAGAAGAAGAAGATGAAGGTGA (SEQ ID NO: 15) |
| R-qPCR-CYCD3-1chr10 | GTTGTTGTTATTATCATCATCAC (SEQ ID NO: 16) |
| F-qPCR-CYCD3; 1-CHR04 | GCAGCACCTTAGTAAACAACTCC (SEQ ID NO: 17) |
| R-qPCR-CYCD3; 1-CHR04 | TCCACGGCTTCTATGCGAGCAC (SEQ ID NO: 18) |
| F-qPCR-CYCD3; 2-CHR17 | GAGATTGAATCATTTAATGCGACC (SEQ ID NO: 19) |
| R-qPCR-CYCD3; 2-CHR17 | GACAGCACCTGGGCTACCCGGT (SEQ ID NO: 20) |
| F-qPCR-CYCD3; 3-CHR05 | GAGGGAGAGACCCATTTGCGTTC (SEQ ID NO: 21) |
| R-qPCR-CYCD3; 3-CHR05 | GTGTCATCCACGGCTTGTCCCTC (SEQ ID NO: 22) |
| Cons6_qRT_F | AGATAGGGAAATGGTGCAGGT (SEQ ID NO: 23) |
| Cons6_qRT_R | CTAATGGCAATTGCAGCTCTC (SEQ ID NO: 24) |
| Cons4_qRT_F | GATCAGCAATTATGCACAACG (SEQ ID NO: 25) |
| Cons4_qRT_R | CCGCCACCATTCAGATTATGT (SEQ ID NO: 26) |

Primers for Copy number
analysis

| | |
|---|---|
| F-CNVref1 | GTGGCGTTGGCAGTGGTACCG (SEQ ID NO: 27) |
| R-CNVref1 | CAGTTTGATGGTCCCCATTAGC (SEQ ID NO: 28) |
| F-CNVref2 | GCATCACAGTGCAATTTAGCTG (SEQ ID NO: 29) |
| R-CNVref2 | CCGCTGAGTTTGCCTTGCTGG (SEQ ID NO: 30) |
| F-KIX8-chr17_CNV | CAACAGCCTCCTTGTCTGCCTCC (SEQ ID NO: 31) |
| R-KIX8-chr17_CNV | TGGTGATGACCAGAAGGGATACC (SEQ ID NO: 32) |

Primers for mapping

| | |
|---|---|
| F-KIX8-chr17 | CCACAAATTCTTACGGAATGTGG (SEQ ID NO: 33) |
| R-KIX8-chr17 | CGCAACAGGTTTAGGAGGCAGAC (SEQ ID NO: 34) |
| F-Glyma06g19660 | CTGGCTTGGCTACACTTGCATC (SEQ ID NO: 35) |
| R-Glyma06g19660 | CACTCACTCGTCACTAGTAGCC (SEQ ID NO: 36) |
| F-Glyma18g40060 | GTGGTTACTGGTCACCCTTGGCAC (SEQ ID NO: 37) |
| R-Glyma18g40060 | GCACAATCTTAGCATTGTTGAATGG (SEQ ID NO: 38) |
| F-Glyma19g44590 | CATGGGTTCTCATGTGTTCATGTG (SEQ ID NO: 39) |
| R-Glyma19g44590 | CGTTCAATACTCCCTCAAGATCAG (SEQ ID NO: 40) |
| F-control-Glyma.02g012600 | ACTTCACCTTCTATGCCCCTG (SEQ ID NO: 41) |
| R-control-Glyma.02g012600 | GTTGGCCAAATCCCAAGACG (SEQ ID NO: 42) |

Primers for construction CRISR/Cas9

| | |
|---|---|
| F-KIX8-Cripsr1 | GATTGGCCTTACGAGTGCGTGAGA (SEQ ID NO: 43) |
| R-KIX8-Cripsr1 | AAACTCTCACGCACTCGTAAGGCC (SEQ ID NO: 44) |
| F-KIX8-Cripsr2 | GATTGCTCCCCGTGGTGGTTCTCA (SEQ ID NO: 45) |
| R-KIX8-Cripsr2 | AAACTGAGAACCACCACGGGGAGC (SEQ ID NO: 46) |
| | The underlined sequences were used to generate |
| | restriction site (BbsI) to ligate them in the |
| | backbone vector (pAtU6-26-SK). |

Primers for CRISPR genotyping

| | |
|---|---|
| F-KIX1CRISPGenotype | TTCTCTCGCTACTCCTCCTACC (SEQ ID NO: 47) |
| R-KIX1CRISPGenotype | GTACTCTGCCTAAGCAACAACCA (SEQ ID NO: 48) |
| F-KIX2CRISPGenotype | GAGTGAGCGAGTGAGCACTGCC (SEQ ID NO: 49) |
| R-KIX2CRISPGenotype | CAAATTCCGCAAGCATTTTGTG (SEQ ID NO: 50) |

Primers for fusing GmKIX1 with GFP

| | |
|---|---|
| F-KIX8ch17MDC83 | AAAAAGCAGGCTCCATGCCGCGCCCAGGGCCAAG |
| | (SEQ ID NO: 51) |
| R-KIX8ch17MDC83 | AAGAAAGCTGGGTCCGAACCTGGCCTACTAGTAAAAC |
| | (SEQ ID NO: 52) |

SSR marker

| | |
|---|---|
| F-KIX-SSR-GRIN | GAGTGAGTGAGCACTGTGTTGTG (SEQ ID NO: 53) |
| R-KIX-SSR-GRIN | ACCAAAACCGCCCCAAGACACTC (SEQ ID NO: 54) |

Primers for sequencing 4.6 kb DNA fragment contained gene GmKIX1

| | |
|---|---|
| F-KIX1-GRIN | GGTACGGACATAGTTCACGATCCC (SEQ ID NO: 55) |
| R-KIX1-GRIN | GATTCCTTGTCCATATCCATTATCC (SEQ ID NO: 56) |

Primers for GUS staining

| | |
|---|---|
| F-proKIX1-GUS | ccaagcttGGTACGGACATAGTTCACGATCCC (SEQ ID NO: |
| | 57) |
| R-proKIX1-GUS | gtcctaggCTTCGAAATGAAAGTGTTAAACTC (SEQ ID NO: |
| | 58) |

Primers for measuring Promoter-LUC activity

| | |
|---|---|
| F-KIX-pro-LucHindIII | ccaagcttGGTACGGACATAGTTCACGATC (SEQ ID NO: |
| | 59) |
| R-KIX-pri-lucNotI | tgcggccgcCTTCGAAATGAAAGTGTTAAAC (SEQ ID NO: |
| | 60) |
| R-del1 | TGTCTGTCTGTTGGGGAGAG (SEQ ID NO: 61) |
| F-del1 | CACTCAATTATTTCTGTCC (SEQ ID NO: 62) |
| R-de12 | GAGAACAATATGGGAAGAAG (SEQ ID NO: 63) |
| R-de12 | CTTGGGGCGGTTTTGGTTTG (SEQ ID NO: 64) |
| R-de13 | TGTCTGTCTGTTGGGGAGAG (SEQ ID NO: 65) |
| R-del3 | CTTGGGGCGGTTTTGGTTTG (SEQ ID NO: 66) |

### Sequence Comparison

Amino acid sequences encoded by the soybean GmKIX genes were obtained from Phytozome (world wide web at phytozome.jgi.doe.gov). These data were further combined with protein sequences from the National Center for Biotechnology Information (NCBI) and PLAZA databases (Van Bel et al., 2017). Evolutionary analyses were conducted in MEGA v.6.06 (Tamura et al., 2013). Protein sequences were aligned using the algorithm MUSCLE, and then compared using the Maximum Likelihood method based on the Jones-Taylor-Thornton (JTT) matrix-based model. Initial trees for the heuristic search were obtained automatically by applying the Neighbor-Joining and BioNJ algorithms to a matrix of pair-wise distances estimated using a JTT model, with 1000 bootstrap replicates.

### Statistical Analysis

Sample means between genotypes or treatments were compared using Student's t-test or one-way ANOVA followed by a post-hoc Tukey's multiple range test. All statistical analyses were performed using SPSS v.24.1.

### Results

Identification of a Fast Neutron (FN) Soybean Mutant with Increased Leaf and Seed Size

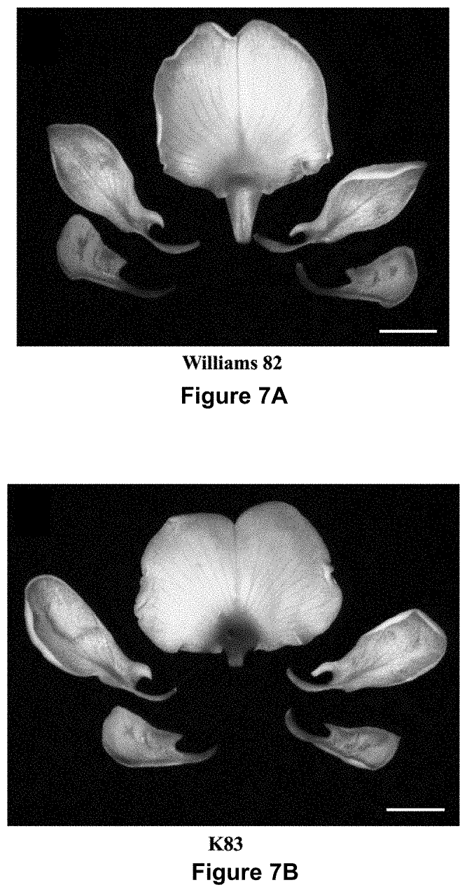

As previously described (Stacey et al., 2016), we conducted a visual screen of a fast neutron irradiated population of G. max cv Williams 82 specifically for lines showing morphological and developmental defects. This screen identified mutant line K83 that produced larger leaves and seeds relative to the parent line. Cotyledon and leaf area measurements showed that the cotyledon and leaf size of the K83 mutant were significantly bigger than wild-type across different early vegetative stages (cotyledon to V4) (FIG. 1A,B). Moreover, the K83 mutant exhibited significantly larger seed size, with 30% higher 100-seed weight compared to wild-type (FIG. 1C,D). In contrast to the above ground parts, the mutant showed similar root fresh weight as the wild-type (FIG. 1E). Flower morphology was also examined, and no obvious differences in floral organ size between the wild-type and mutant flowers were observed (FIG. 7). Since organ size is determined by cell size and number, these parameters were measured in K83 and wild-type cotyledon epidermal cells. The epidermal cell number was significantly increased in fully expanded 2-wk-old cotyledons of K83 compared to the wild-type (FIG. 1F,G). In contrast to total cell number, stomatal index and guard cell length were not significantly different in expanded cotyledons between the mutant and wild-type (FIG. 1H,I). These results suggest that cell proliferation rather than cell expansion was responsible for the increased cotyledon and leaf size in the mutant.

### Deletion Encoding GmKIX8-1 is the Causative Genetic Lesion for Increased Organ Size

K83 was back-crossed to Williams 82 (parental cultivar) and segregating BC1F2 progenies were phenotyped for leaf and seed weight. The big leaf phenotype appeared in c. 25% (63 wild-type:19 big leaves ratio) of the BC1F2 population, which fits the 3:1 ratio (v2 test, P=0.702) for a monogenic recessive allele. Interestingly, in contrast to the big leaf phenotype, c. 75% of the BC1F2 progenies produced big seeds, with all big leaf plants showing the big seed phenotype. The segregation data (24 wild-type:59 big seeds ratio) fit the 1 wild-type:3 mutant ratio (v2 test, P=0.41) consistent with a single, dominant allele. Similar segregation ratios were found in the BC1F3 progenies (Table 1). This discrepancy in the inheritance patterns of the increased leaf and seed phenotypes is interesting and would indicate that the phenotypes are caused by independent genetic lesions or, alternatively, by a single lesion that acts differently in somatic and embryonic tissues.

To further elucidate the genetics underlying the mutant phenotypes, CGH analysis was performed on K83 genomic DNA to identify FN-induced lesions in the mutant genome. Five deleted DNA segments were identified, one each in chromosomes 6, 17 and 19 and two in chromosome 18 (FIG. 2A; FIG. 2a). To determine which of the deletions is responsible for the observed phenotypes, specific primer pairs were used to examine the segregation of each deletion in BC1F3 progenies. From these genetic mapping experiments, only the deletion on chromosome 17 was found to co-segregate with both the leaf and seed phenotypes (FIG. 2) indicating that the causative gene(s) is (are) encoded in the chromosome 17 deleted region.

Based on the annotated soybean genome, there are 48 predicted genes located in the deleted region of chromosome 17 (Table 3).5

TABLE 3

| | Gene Start | | | |
|---|---|---|---|---|
| Gene Name | (bp) | PFAM Description | Panther ID | GO ID |
| Glyma.17G112700 | 8895386 | ABC transporter | PTHR19211 | GO: 0016887 |
| Glyma.17G112800 | 8907367 | GmKIX8-1 | PTHR35300 | — |
| Glyma.17G112900 | 8917773 | Plastocyanin-like domain | PTHR33021 | GO: 0009055 |
| Glyma.17G113000 | 8922288 | Plastocyanin-like domain | PTHR33021 | GO: 0009055 |
| Glyma.17G113000 | 8926007 | Plastocyanin-like domain | PTHR33021 | GO: 0009055 |
| Glyma.17G113200 | 8928093 | Glycosyl hydrolases | PTHR32227 | GO: 0005975 |
| | | family 17 | | |
| Glyma.17G113300 | 8941948 | | P1HR35742 | |
| Glyma.17G113400 | 8967967 | HMG-box domain | PTHR31675 | GO: 0007275 |
| Glyma.17G113500 | 8985556 | Nucleoside transporter | PTHR10332 | GO: 0016021 |
| Glyma.17G113600 | 8989211 | | P1HR26312 | |
| Glyma.17G113700 | 8997933 | Kinase-like | PTHR24343 | GO: 0007165 |
| Glyma.17G113800 | 9007837 | | PTHR33109 | |
| Glyma.17G113900 | 9011720 | F-box-like | PTHR24006 | GO: 0005515 |
| Glyma.17G114000 | 9034387 | Protein of unknown | PTHR33373 | |
| | | function (DUF4050) | | |
| Glyma.17G114100 | 9042968 | Ribosomal protein L36 | PTHR18804 | GO: 0006412 |
| Glyma.17G114200 | 9048733 | | | |
| Glyma.17G114300 | 9049860 | | P1HR33564 | |
| Glyma.17G114400 | 9054340 | Rab-GTPase-TBC | PTHR22957 | |
| | | domain | | |
| Glyma.17G114500 | 9062347 | AP2 domain | PTHR31194 | GO: 0006355 |
| Glyma.17G114600 | 9071697 | | PTHR22773 | |
| Glyma.17G114700 | 9074437 | SPX domain | PTHR10783 | |
| Glyma.17G114800 | 9088001 | | PTHR15852 | |
| Glyma.17G114900 | 9090352 | Protein of unknown | PTHR35550 | |
| | | function (DUF3119) | | |
| Glyma.17G115000 | 9095427 | Methyltransferase FkbM | PTHR34203 | |
| | | domain | | |
| Glyma.17G115100 | 9099718 | Core-2/I-Branching | P1HR19297 | GO: 0016020 |
| | | enzyme | | |
| Glyma.17G115200 | 9110887 | Triose-phosphate | PTHR11132 | |
| | | Transporter family | | |
| Glyma.17G115300 | 9122203 | POT family | PTHR11654 | GO: 0016020 |
| Glyma.17G115400 | 9137066 | Ribosomal protein | PTHR23105 | |
| | | L7Ae/L30e/S12e/Gadd45 | | |
| | | family | | |
| Glyma.17G115500 | 9140214 | Ribosome biogenesis | PTHR17602 | GO: 0042254 |
| | | regulatory protein | | |
| | | (RRS1) | | |
| Glyma.17G115600 | 9144108 | Mitochondrial carrier | PTHR24089 | |
| | | protein | | |
| Glyma.17G115700 | 9147302 | Helicase conserved C- | PTHR24031 | GO: 0005524 |
| | | terminal domain | | |
| Glyma.17G115800 | 9153096 | | PTHR31134 | |
| Glyma.17G115900 | 9163290 | Glyoxalase/Bleomycin | PTHR10374 | |
| | | resistance | | |
| | | protein/Dioxygenase | | |
| | | superfamily | | |
| Glyma.17G116000 | 9171575 | Dynein light chain type 1 | PTHR11886 | GO: 0007017 |
| Glyma.17G116100 | 9177090 | | PTHR33730 | |
| Glyma.17G116200 | 9181066 | Zinc finger C- | PTHR15725 | GO: 0046872 |
| | | x8-C-x5-C-x3- | | |
| | | H type (and similar) | | |
| Glyma.17G116300 | 9184627 | F-box domain | PTHR32133 | GO: 0005515 |
| Glyma.17G116400 | 9188071 | Oxidoreductase FAD- | PTHR19370 | GO: 0055114 |
| | | binding domain | | |
| Glyma.17G116500 | 9199427 | Asp/Glu/Hydantoin | PTHR21198 | GO: 0036361 |
| | | racemase | | |
| Glyma.17G116600 | 9200382 | NADH pyrophosphatase | PTHR22769 | GO: 0046872 |
| | | zinc ribbon domain | | |
| Glyma.17G116700 | 9206095 | Zinc finger C-x8-C-x5- | PTHR12547 | GO: 0046872 |
| | | C-x3-H type (and | | |
| | | similar) | | |
| Glyma.17G116800 | 9214170 | Zn-finger in Ran binding | PTHR23111 | GO: 0008270 |
| | | protein and others | | |
| Glyma.17G116900 | 9223358 | Zn-finger in Ran binding | PTHR23111 | GO: 0008270 |
| | | protein and others | | |
| Glyma.17G117000 | 9226104 | PPR repeat | PTHR24015 | |
| Glyma.17G117100 | 9228211 | Early nodulin 93 | PTHR33605 | |
| | | ENOD93 protein | | |
| Glyma.17G117200 | 9234326 | Early nodulin 93 | PTHR33605 | |
| | | ENOD93 protein | | |
| Glyma.17G117300 | 9242281 | Protein tyrosine kinase | PTHR24351 | GO: 0006468 |
| Glyma.17G117400 | 9251664 | | PTHR15852 | |

One of the genes within this predicted deletion is Glyma.17G112800 (designated as GmKIX8-1), encoding a putative protein with high sequence similarity to AtKIX8 (At3g24150) (FIG. 8). Since genetic defects in AtKIX8 are known to result in increased seed and leaf size in Arabidopsis (Gonzalez et al., 2015; Liu et al., 2020), we hypothesized that the GmKIX8-1 deletion is the causative genetic lesion responsible for the mutant phenotypes in K83. Gene expression analysis of leaves showed that the expression of GmKIX8-1 was detected in leaves of wild-type plants but not in the K83 mutant, further indicating that the GmKIX8-1 gene was indeed deleted in the mutant (FIG. 2C). Soybean, being an allotetraploid, encodes a second, paralogous GmKIX8 gene, Glyma.13G158300 (GmKIX8-2), which showed similar expression to GmKIX8-1 in wild-type leaves. GmKIX8-2 expression was not affected in K83 plants, consistent with our CGH results indicating that only GmKIX8-1 was deleted in the mutant genome (FIG. 2C).

Consistent with the co-segregation of the big leaf phenotype with the Chr. 17 deletion encoding GmKIX8-1 (FIG. 2B), copy number analysis of GmKIX8-1 in BC1F3 plants showed that homozygous mutants (i.e., 1097-2, -5, -10 and -22 in FIG. 2D) produced larger leaves and seeds relative to wild-type (i.e., 1097-1, -3, -7, -16 and -28 in FIG. 2D; Table 1). Moreover, plants that were heterozygous for the GmKIX8-1 lesion (i.e., 1097-4, -6 and -9 in FIG. 2D; Table 1) produced big seeds, but the leaves showed a wild-type phenotype. These results indicate that the leaf and seed phenotypes associated with the K83 mutant line are caused by a single genetic lesion, most likely the deletion of GmKIX8-1. Moreover, the observed dominant seed trait is likely due to haploid insufficiency, that is, the decreased dosage of GmKIX8-1 in heterozygous plants.

### CRISPR Cas9-Induced Mutation in GmKIX8-1 Resulted in Increased Organ Size

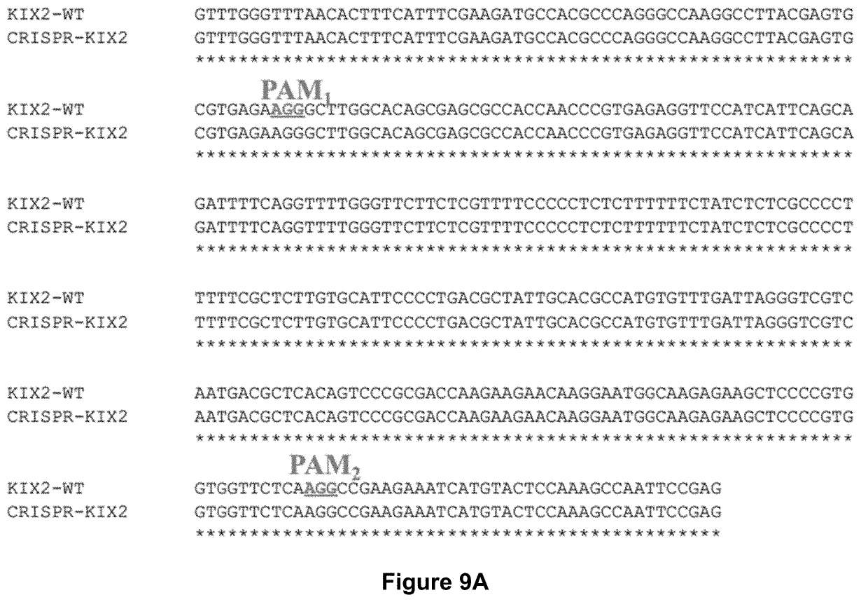

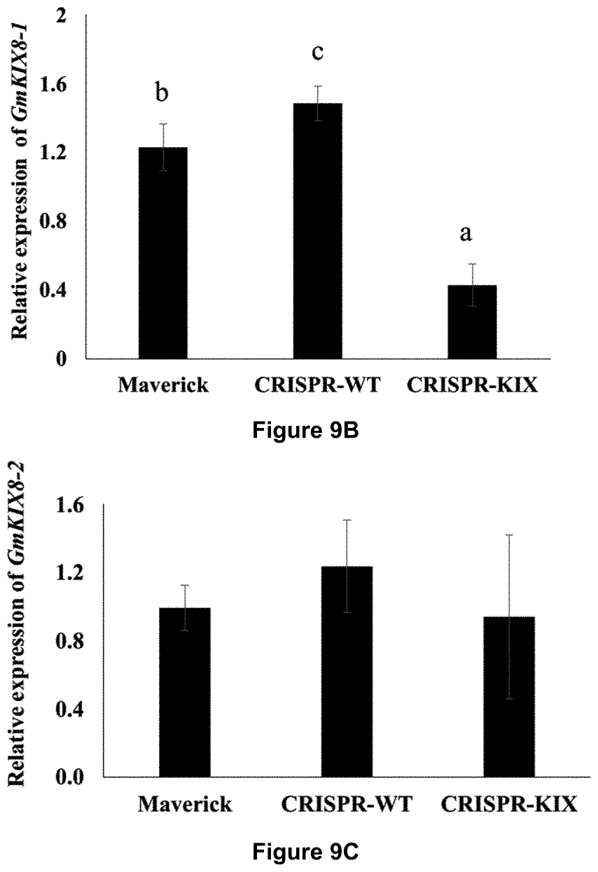

As previously mentioned, the deletion in Chr. 17 encodes 48 genes, including GmKIX8-1. In order to confirm that the K83 mutant phenotypes were solely due to the deletion of GmKIX8-1 rather than to co-deleted gene(s) on chromosome 17, we constructed GmKIX8-1 knockout lines via CRISPR/Cas9 genome editing. A CRISPR/Cas9 binary vector containing two guide RNAs (dual gRNAs) targeting the first (target 1) and second (target 2) exons of GmKIX8-1 was constructed (FIG. 3A) and transformed into soybean (Maverick cultivar). We identified one T0 plant, C7-19, harboring a GmKIX8-1 deletion as indicated by the presence of higher mobility PCR amplicons (mutant band) using primers flanking the two target sites (F and R in FIG. 3A). The gel electrophoretic mobility of the mutant band is consistent with the predicted deletion of DNA sequences (˜220 bps) between targets 1 and 2. Genotyping of T1 progenies showed inheritance of the mutant PCR band (FIG. 3B). Sanger sequencing of PCR amplicons (upper and lower bands in FIG. 3B) from C7-19 confirmed that the mutant was heterozygous for the GmKIX8-1 lesion and that sequences between the protospacer adjacent motif (PAM) sites were deleted (FIG. 3C). Subsequent sequencing of PCR amplicons from a homozygous T1 gmkix8-1 plant (CRISPR-KIX, T1-1 in FIG. 3B) and a homozygous wild-type plant (CRISPR-WT, T1-2 in FIG. 3B) were consistent with the sequencing results from the T0 plant. Although the gRNAs were designed to target GmKIX8-1, it is still possible that GmKIX8-2 was off-targeted in C7-19 due to the high DNA sequence identity between the GmKIX8 genes. Therefore, genomic DNA was amplified using GmKIX8-2-specific primers, and subsequent sequencing showed no mutations in GmKIX8-2 (lower panel FIG. 3B; FIG. 9A). In addition, the expression levels of GmKIX8-2 were similar in Maverick, CRISPR-WT and CRISPR-KIX T1 plants (FIG. 9C).

Consistent with the FN K83 Mutant, Phenotypic Analysis of

T1 segregating progenies derived from the CRISPR C7-19 line showed that increased leaf size co-segregated with the homozygous gmkix8-1 mutation (FIG. 3D,F; FIG. 10A,B). The number of abaxial epidermal cells was also significantly increased in the CRISPR-KIX line (FIG. 10D-G). Importantly, roughly 75% big seeds were produced from the heterozygous populations from the CRISPR/Cas9 T2 generation (25 wild type. 65 big seed ratio, v2 test, P=0.543) and the homozygous gmkix8-1 CRISPR-Cas9 mutant plants produced seeds c. 30% heavier than controls (FIG. 3E,G; FIG. 10H). However, total seed numbers per plant in the heterozygous and homozygous gmkix8-1 CRISPR mutants were significantly reduced compared to control (FIG. 10I). Taken together, these results support our hypothesis that the loss of function of GmKIX8-1 is the genetic basis for the increased leaf and seed weight phenotypes of the K83 FN mutant line. These results also showed a haplo-insufficient role of GmKIX8-1 for regulating seed size, but not leaf size, in soybean.

### Expression, Sub-Cellular Localization and Downstream Targets of GmKIX8-1

Given its close phylogenetic relationship to the Arabidopsis AtKIX8 (FIG. 8), we predicted that GmKIX8-1 also functions as a component protein of a transcriptional repressor complex involved in controlling cell division in soybean. To determine whether GmKIX8-1, like AtKIX8, is localized in the nucleus, we fused the C-terminus of GmKIX8-1 to GFP and transiently expressed the fusion protein from a CaMV 35S promoter in tobacco leaves. Confocal microscopy to visualize GFP expression in infiltrated tissues showed that the GmKIX8-1-GFP fusion protein was indeed localized to the nucleus (FIG. 4A,B). We also determined the expression pattern of GmKIX8-1 in different tissues, such as seeds, leaves, roots, shoot tips, nodules and flowers. We found that GmKIX8-1 was expressed in all the tissues analyzed, with the highest levels of expression in shoot apical meristems and opened flowers, whereas it was less expressed in root and nodule tissues (FIG. 11A,B).

AtKIX8 and AtKIX9 act in a repressor complex with AtPPD2 to repress the expression of D3-type cyclins, which are important in cell number determination in developing Arabidopsis leaves (Gonzalez et al., 2015; Baekelandt et al., 2018). To determine whether GmKIX8-1 functions in a similar manner in soybean, we determined the expression levels of a D3-type cyclin gene, GmCYCD3;1-10 (Glyma.10G263500), in the shoot tips of wild-type and CRISPR-KIX mutant plants. Indeed, we found that GmCYCD3;1-10 was significantly upregulated in the gmkix8-1 mutant compared to wild-type plants (FIG. 4C). By contrast, the expression levels of other CYCD3 genes, namely GmCYCD3;2-17 (Glyma.17G167700) and GmCYCD3;3-10 (Glyma.05G097300), did not change in the mutant. Although GmCYD3;3-10 was not differentially expressed in gmkix8-1, increased expression of another GmCYD3;3 gene, Glyma.04g042000, was reported in soybean plants where PPD genes were targeted by microRNA (Ge et al, 2016). The soybean orthologs of the Arabidopsis GROWTH REGULATING FACTOR1, another predicted downstream target genes of the AtKIX-AtPPD repressor complex, GRF1-10 (Glyma.10G164100) and GRF1-20 (Glyma.20G226500), also showed comparable expression patterns in the shoot tips of CRISPR-WT and CRISPR-KIX plants (FIG. 4D,E). Moreover, there were no changes in the expression levels of GmBS1 and GmBS2, the soybean orthologs of AtPPD1 (Ge et al., 2016), in the gmkix8-1 homozygous mutant. These results suggest that GmKIX8-1, similar to AtKIX8/9 proteins, controls cell proliferation by repressing the expression of D3-type cyclins.

Soybean Genotypes Encoding QTL qSw17-1 for Big-Seeded Phenotype have Reduced GmKIX8-1 Expression

Given the importance of seed weight in soybean yield and food improvement, a number of QTLs related to seed weight have been identified (world wide web at soybase.org/). A major seed weight QTL, qSw17-1, was previously mapped in several soybean populations (Hoeck et al., 2003; Panthee et al., 2005; Liu et al., 2007; Teng et al., 2008; Kim et al., 2010; Liu et al., 2013; Kato et al., 2014; Yan et al., 2017; Liu et al., 2018) (FIG. 5A). Interestingly, the Chr. 17 deletion encoding GmKIX8-1 in the K83 FN mutant overlaps the mapped location of QTL qSw17-1, and heterozygous and homozygous gmkix8-1 mutants produced big seeds (FIG. 5A). Therefore, we hypothesized that the GmKIX8-1 gene lesion could be responsible for the increased seed weight associated with QTL qSw17-1. To test this hypothesis, we obtained three big seeded plant introductions (PIs), Tachinagaha-PI561396 (Maturity Group (MG) 4), Keunolkong1-PI597483 (MG5) and Keunolkong2 PI594021 (MG1), which were used in mapping QTL qSw17-1 (Kim et al., 2010; Kato et al., 2014). The three PI lines and gmkix8-1 mutants were planted in the field, and 100 seed weights of mature seeds were measured. As wild-type controls, Williams 82 and Maverick were also grown and phenotyped. The data we obtained for PI561396 and PI597483 showed that the seeds produced by these PI lines gave higher 100-seed weight compared to controls and were comparable to those produced by K83 FN mutants (FIG. 5B,C; FIG. 12). However, PI594021 did not grow well under the photoperiod conditions in Columbia, Mo., and we were not able to obtain reliable 100-seed weight data on this cultivar. The epidermal cell number (mm-2) of fully expanded 2-week-old cotyledons of PI597483 was significantly increased compared to the wild-type (FIG. 5D,E). However, unlike K83, there was no obvious difference in leaf size between the PI597483 with controls under glasshouse conditions (FIG. 12C).

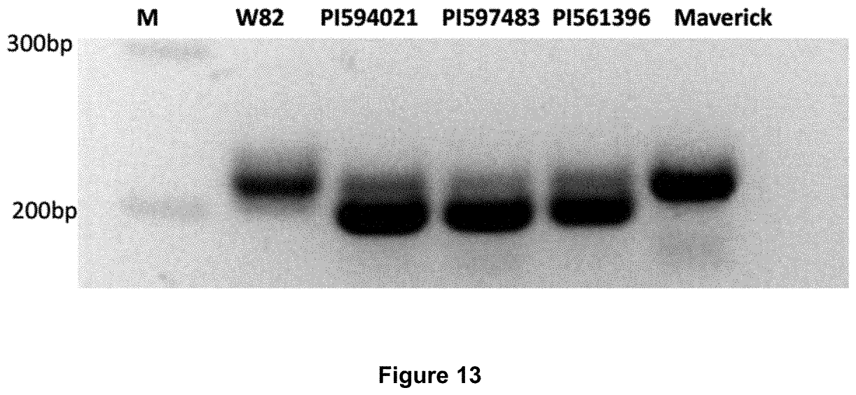

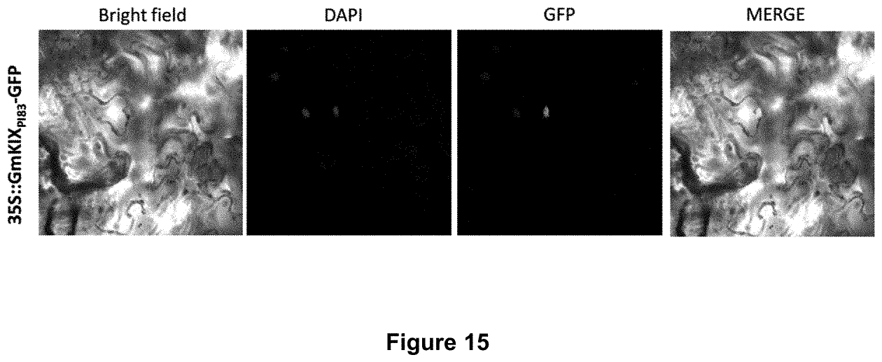

A 4.5 kb genomic fragment from the PIs and the reference cultivar Williams 82 containing 1.6 kb 5′UTR and GmKIX8-1 genomic sequences was cloned and sequenced. The sequencing results identified a total of 30 polymorphisms in the GmKIX8-1 sequences between the PIs and Williams 82, with 22 polymorphisms common in all PIs (FIG. 17). Four small deletions were found in the promoter regions of all PIs, of which two were the tandem repeated sequences CGC and GT located at −177 to −174 and −104 to −92 relative to the ATG start site (FIG. 6A; FIG. 17). More importantly, an SSR marker was developed outside of these two deleted fragments, which can be used to distinguish the normal (e.g. W82, Maverick) and big-seeded cultivars harboring QTL qSw17-1 (e.g. PI594021, PI597483, and PI561396) (FIG. 13). Eight SNPs were detected in exon four, six of which were nonsynonymous poly-morphisms (FIG. 6A, FIG. 14), none of which changed the subcellular localization of GmKIX8-1 (FIG. 15). Lastly, we found a two bp insertion at 86 bps after the stop codon of GmKIX8-1 in all three PIs (FIG. 6A).

To determine whether the GmKIX8-1 promoter deletions negatively affect the expression of GmKIX8-1, we analyzed the expression of GmKIX8-1 and GmCYCD3;1-10 in the shoot tips of PI597483, Williams 82 and Maverick. The results showed that the expression of GmKIX8-1 was significantly downregulated in PI597483 compared to the wild-type controls. Consistent with the downregulated expression of GmKIX8-1, the expression of GmCYCD3;1-10 was significantly upregulated in PI597483 (FIG. 6B,C). We next used a dual-luciferase assay to compare the GmKIX8-1 promoter activity encoded in the Williams 82 (pGmKIX8-1-WT) and PI597483 (pGmKIX8-1-PI597483).

The results showed that, although both pGmKIX8-1-WT and pGmKIX8-1-PI597483 promoter sequences activated LUC expression, the LUC activity driven by pGmKIX8-1-PI597483 was significantly lower than pGmKIX8-1-WT (FIG. 6D), indicating that the two tandem repeats (CGC and GT), which were absent in pGmKIX8-1-PI597483, are necessary for optimum expression of GmKIX8-1. Interestingly, a MEME motif search (Bailey et al., 2009) revealed that the dinucleotide GT tandem repeats are a part of the cis-regulatory sequences in the GmKIX8-1 promoter region (FIG. 16). We next constructed three promoters lacking either the CGC (pGmKIX8-1(del1)) (SEQ ID NO: 69) or GT (pGmKIX8-1(del2)) (SEQ ID NO: 70) or both (pGmKIX8-1(del3)) (SEQ ID NO: 71) tandem repeats to determine their importance in GmKIX8-1 expression (FIG. 6D). These mutant promoters were used to drive the expression of LUC in dual-luciferase assays. The LUC activity driven by pGmKIX8-1(del1) or pGmKIX8-1(del2) was slightly reduced but not statistically different compared to pGmKIX8-1 (WT). Significantly reduced LUC activity, as compared to pGmKIX8-1(WT), was observed only when both tandem repeats were deleted (i.e., in pGmKIX8-1(del3); FIG. 6E).

In summary, examination of the promoter and coding sequences of GmKIX8-1 in three big-seeded PI lines harboring QTL qSw17-1 identified several polymorphisms, including two common deletions in the promoter region, when compared to the wild-type-seeded Williams 82 sequences. The expression of GmKIX8-1 is downregulated in PI597483, due to the promoter deletions as indicated by our dual LUC promoter activity assay results. Consistent with GmKIX8-1 downregulation, the expression of GmCYCD3;1-10 was increased in the PIs. Together with the previously discussed gene dosage effect of GmKIX8-1 on seed weight, these results indicate that the big seeded phenotype associated with QTL qSw17-1 is due to reduced GmKIX8-1 expression.

The breadth and scope of the present disclosure should not be limited by any of the above-described exemplary embodiments, but should be defined only in accordance with the following claims and their equivalents.

The present invention can be defined in any of the following numbered embodiment paragraphs:

1. A method for obtaining a soybean plant comprising in its genome at least one genetic locus that comprises a genotype associated with a large-seed phenotype, the method comprising the steps of:

a. genotyping one or more soybean plants with respect to a genetic locus comprising the soybean GmKIX8-1 gene Glyma.17G112800; and

b. selecting based on said genotyping of said genetic locus a soybean plant comprising a genotype associated with a large-seed phenotype.

2. The method for obtaining a soybean plant of embodiment 1, wherein the genetic locus is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the soybean GmKIX8-1 gene Glyma.17G112800.
3. The method for obtaining a soybean plant of embodiment 1 or 2, wherein the genetic locus consists of the soybean GmKIX8-1 gene Glyma.17G112800.
4. The method for obtaining a soybean plant of any one of embodiments 1 to 3, wherein the genotype associated with the large-seed phenotype is a deletion of or within the soybean GmKIX8-1 gene Glyma.17G112800.
5. The method for obtaining a soybean plant of embodiment 4, wherein the genotype associated with the large-seed phenotype is a deletion within the protein coding region and/or the promoter region of the soybean GmKIX8-1 gene Glyma.17G112800,

optionally, (i) wherein said promoter region deletion comprises a deletion of the tandem repeated sequence CGC located at −177 to −174 relative to the GmKIX8-1 gene ATG start site;

optionally, (ii) wherein said promoter region deletion comprises a deletion of the tandem repeated sequence GT located at −104 to −92 relative to the GmKIX8-1 gene ATG start site; or

optionally, wherein said promoter region deletion comprises both the deletions of (i) and (ii).

6. The method for obtaining a soybean plant of any one of embodiments 1 to 5, wherein the genotype associated with the large-seed phenotype comprises at least one allele associated with the large-seed phenotype identified in the alignment of soybean GmKIX8-1 genes in FIG. 17.
7. The method for obtaining a soybean plant of embodiment 6, wherein the genotype associated with the large seed phenotype comprises at least one of the two 3′ most deletions in the promoter region.
8. The method for obtaining a soybean plant of embodiment 7, wherein the genotype associated with the large seed phenotype comprises at least the two 3′ most deletions in the promoter region.
9. The method for obtaining a soybean plant of any one of embodiments 1 to 8, wherein said selected soybean plant or a progeny thereof exhibits the large-seed phenotype.
10. The method for obtaining a soybean plant of any one of embodiments 1 to 9, further comprising crossing the selected soybean plant having in its genome the genetic locus comprising a genotype associated with a large-seed phenotype with a soybean plant that does not have in its genome a genetic locus comprising a genotype associated with a large-seed phenotype to produce a progeny soybean plant comprising in its genome a genetic locus comprising a genotype associated with a large-seed phenotype.
11. The method for obtaining a soybean plant of embodiment 10, wherein the progeny soybean plant exhibits the large-seed phenotype.
12. The method for obtaining a soybean plant of embodiment 10 or 11, wherein the selected soybean plant having in its genome the genetic locus comprising a genotype associated with a large-seed phenotype is crossed with a soybean plant that does not have in its genome a genetic locus comprising a genotype associated with a large-seed phenotype to produce a population of soybean plants comprising in their genomes a genetic locus comprising a genotype associated with a large-seed phenotype.
13. The method for obtaining a soybean plant of embodiment 12, wherein the population of soybean plants produced comprises a plurality of soybean plants that exhibit the large-seed phenotype.
14. The method for obtaining a soybean plant of embodiment 13, wherein at least 50%, at least 60%, at least 70%, at least 75%, at least 80%, at least 85%, at least 90%, at least 95%, at least 96%, at least 97%, at least 98%, at least 99%, or 100% of the soybean plants in the population of soybean plants produced exhibit the large-seed phenotype.
15. The method for obtaining a soybean plant of any one of embodiments 1 to 14, wherein the genotyping is done with a SSR marker primer pair comprising the sequences of F-KIX-SSR-GRIN GAGTGAGTGAGCACTGTGTTGTG (SEQ ID NO: 53) and R-KIX-SSR-GRIN ACCAAAACCGCCCCAAGACACTC (SEQ ID NO: 54), a primer pair comprising the sequences of F-KIX1CRISPGenotype TTCTCTCGCTACTCCTCCTACC (SEQ ID NO: 47) and R-KIX1CRISPGenotype GTACTCTGCCTAAGCAACAACCA (SEQ ID NO: 48), a primer pair comprising the sequences of F-KIX2CRISPGenotype GAGTGAGCGAGTGAGCACTGCC (SEQ ID NO: 49) and F-KIX2CRISPGenotype CAAATTCCGCAAGCATTTTGTG (SEQ ID NO: 50), or a primer pair comprising the sequences of F-KIX1-GRIN GGTACGGACATAGTTCACGATCCC (SEQ ID NO: 55) and R-KIX1-GRIN GATTCCTTGTCCATATCCATTATCC (SEQ ID NO: 56).
16. The method for obtaining a soybean plant of any one embodiments 1 to 15, wherein the genetic locus comprising the GmKIX8-1 gene Glyma.17G112800I that comprises a genotype associated with a large-seed phenotype has been gene edited.
17. A method for producing a soybean plant comprising in its genome an introgressed genetic locus comprising a genotype associated with a large-seed phenotype, the method comprising the steps of:

a. crossing a first soybean plant with a genotype associated with a large-seed phenotype in a first polymorphic genetic locus comprising the soybean GmKIX8-1 gene Glyma.17G112800 with a second soybean plant comprising a genotype not associated with a large-seed phenotype in the polymorphic genetic locus comprising Glyma.17G112800 and at least one second polymorphic locus that is linked to the genetic locus comprising Glyma.17G112800 and that is not present in said first soybean plant to obtain a population segregating for the large-seed phenotype polymorphic locus and said linked second polymorphic locus;

b. genotyping for the presence of at least two polymorphic nucleic acids in at least one soybean plant from said population, wherein a first polymorphic nucleic acid is located in said genetic locus comprising Glyma.17G112800 and wherein a second polymorphic amino acid is the linked second polymorphic locus not present in said first soybean plant; and

c. selecting a soybean plant comprising a genotype associated with the large-seed phenotype and the at least one linked marker found in said second soybean plant that does not comprise a large-seed phenotype locus but not found in said first soybean plant, thereby obtaining a soybean plant comprising in its genome an introgressed large-seed phenotype locus.

18. The method for producing a soybean plant of embodiment 17, wherein the genetic locus comprising a genotype associated with a large-seed phenotype is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the GmKIX8-1 gene Glyma.17G112800.
19. The method for producing a soybean plant of embodiment 17 or 18, wherein the genetic locus comprising a genotype associated with a large-seed phenotype consists of the GmKIX8-1 gene Glyma.17G112800.
20. The method for producing a soybean plant of any one of embodiments 17 to 19, wherein the genotype associated with the large-seed phenotype is a deletion of or within the GmKIX8-1 gene Glyma.17G112800.
21. The method for producing a soybean plant of embodiment 20, wherein the genotype associated with the large-seed phenotype is a deletion within the protein coding region and/or promoter region of the GmKIX8-1 gene Glyma.17G112800,

optionally, (i) wherein said promoter region deletion comprises a deletion of the tandem repeated sequence CGC located at −177 to −174 relative to the GmKIX8-1 gene ATG start site;

optionally, (ii) wherein said promoter region deletion comprises a deletion of the tandem repeated sequence GT located at −104 to −92 relative to the GmKIX8-1 gene ATG start site; or optionally, wherein said promoter region deletion comprises both the deletions of (i) and (ii).

22. The method for producing a soybean plant of any one of embodiments 17 to 21, wherein the genotype associated with the large-seed phenotype comprises at least one allele associated with the large-seed phenotype identified in the alignment of GmKIX8-1 genes in FIG. 17.
23. The method for producing a soybean plant of embodiment 22, wherein the genotype associated with the large seed phenotype comprises at least one of the two 3′ most deletions in the promoter region.
24. The method for producing a soybean plant of embodiment 23, wherein the genotype associated with the large seed phenotype comprises at least the two 3′ most deletions in the promoter region.
25. The method for producing a soybean plant of any one of embodiments 17 to 24, wherein the population of soybean plants produced comprises a plurality of soybean plants that exhibit the large-seed phenotype.
26. The method for producing a soybean plant of any one of embodiments 17 to 25, wherein said second linked polymorphic locus is detected with a marker that is located within about 1 Mb, 500, 100, 40, 20, 10, or 5 kilobases (Kb) of Glyma.17G112800.
27. The method for producing a soybean plant of any one of embodiments 17 to 26, wherein the genotyping of the first polymorphic nucleic acid located in the genetic locus comprising Glyma.17G112800 is done with a SSR marker primer pair comprising the sequences of F-KIX-SSR-GRIN GAGTGAGTGAGCACTGTGTTGTG (SEQ ID NO: 53) and R-KIX-SSR-GRIN ACCAAAACCGCCCCAAGACACTC (SEQ ID NO: 54), a primer pair comprising the sequences of F-KIX1CRISPGenotype TTCTCTCGCTACTCCTCCTACC (SEQ ID NO: 47) and R-KIX1CRISPGenotype GTACTCTGCCTAAGCAACAACCA (SEQ ID NO: 48), a primer pair comprising the sequences of F-KIX2CRISPGenotype GAGTGAGCGAGTGAGCACTGCC (SEQ ID NO: 49) and F-KIX2CRISPGenotype CAAATTCCGCAAGCATTTTGTG (SEQ ID NO: 50), or a primer pair comprising the sequences of F-KIX1-GRIN GGTACGGACATAGTTCACGATCCC (SEQ ID NO: 55) and R-KIX1-GRIN GATTCCTTGTCCATATCCATTATCC (SEQ ID NO: 56).
28. The method for producing a soybean plant of any one of embodiments 17 to 27, wherein the polymorphic genetic locus comprising the GmKIX8-1 gene Glyma.17G112800I having a genotype associated with a large-seed phenotype has been gene edited.
29. A soybean plant comprising an introgressed genetic locus comprising a genotype associated with a large-seed phenotype in a genomic region comprising the soybean GmKIX8-1 gene Glyma.17G112800I, wherein at least one marker linked to the introgressed large-seed phenotype genetic locus found in said soybean plant is characteristic of germplasm comprising a non-large-seed genetic locus but is not associated with germplasm comprising the large-seed phenotype genetic locus.
30. The soybean plant of embodiment 29, wherein the genetic locus is a genomic region between any of about 1 Mb, 500 kb, 100 kb, 50 kb, or 10 kb telomere proximal and any of about
1 Mb, 500 kb, 100 kb, 50 kb, or 10 kb centromere proximal of the GmKIX8-1 gene Glyma.17G112800I.
31. The soybean plant of embodiment 29 or 30, wherein the genetic locus consists of the GmKIX8-1 gene Glyma.17G112800I.
32. The soybean plant of any one of embodiments 29 to 31, wherein the genotype associated with the large-seed phenotype is a deletion of or within the GmKIX8-1 gene Glyma.17G112800.
33. The soybean plant of embodiment 32, wherein the genotype associated with the large-seed phenotype is a deletion within the protein coding region and/or promoter region of the GmKIX8-1 gene Glyma.17G112800,

optionally, (i) wherein said promoter region deletion comprises a deletion of the tandem repeated sequence CGC located at −177 to −174 relative to the GmKIX8-1 gene ATG start site;

optionally, (ii) wherein said promoter region deletion comprises a deletion of the tandem repeated sequence GT located at −104 to −92 relative to the GmKIX8-1 gene ATG start site; or optionally, wherein said promoter region deletion comprises both the deletions of (i) and (ii).

34. The soybean plant of any one of embodiments 29 to 33, wherein the genotype associated with the large-seed phenotype comprises at least one allele associated with the large-seed phenotype identified in the alignment of GmKIX8-1 genes in FIG. 17.
35. The soybean plant of embodiment 34, wherein the genotype associated with the large-seed phenotype comprises at least one of the two 3′ most deletions in the promoter region.
36. The soybean plant of embodiment 36, wherein the genotype associated with the large seed phenotype comprises at least the two 3′ most deletions in the promoter region.
37. The soybean plant of any one of embodiments 29 to 36, wherein the soybean plant exhibits the large-seed phenotype.
38. The soybean plant of any one embodiments 29 to 37, wherein the genetic locus comprising the GmKIX8-1 gene Glyma.17G112800I having a genotype associated with a large-seed phenotype has been gene edited.
39. The soybean plant of any one of embodiments 29 to 38, made by the method for producing a soybean plant of any one of embodiments 17 to 28.
40. A method of identifying a soybean plant that comprises a genotype associated with a large-seed phenotype, the method comprising:

(a) genotyping a soybean plant in at least one polymorphic genetic locus associated with a large-seed phenotype for the presence of a genotype associated with a large-seed phenotype, wherein the genetic locus comprises the GmKIX8-1 gene Glyma.17G112800, and

(b) denoting based on the genotyping that said soybean plant comprises a genotype associated a large-seed phenotype.

41. The method of identifying a soybean plant of embodiment 40, wherein said method further comprises the step of selecting said denoted plant from a population of plants.
42. The method of identifying a soybean plant of embodiment 40 or 41, wherein said soybean plant comprising in its genome the genotype associated with a large-seed phenotype exhibits a large-seed phenotype.
43. The method of identifying a soybean plant of any one of embodiments 40 to 42, wherein the genetic locus comprising a genotype associated with a large-seed phenotype is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the soybean GmKIX8-1 gene Glyma.17G112800.
44. The method of identifying a soybean plant of any one of embodiments 40 to 43, wherein the genetic locus comprising a genotype associated with a large-seed phenotype consists of the GmKIX8-1 gene Glyma.17G112800.
45. The method of identifying a soybean plant of any one of embodiments 40 to 44, wherein the genotype associated with the large seed phenotype is a deletion of or within the GmKIX8-1 gene Glyma.17G112800.
46. The method of identifying a soybean plant of embodiment 45, wherein the genotype associated with the large seed phenotype is a deletion within the protein coding region and/or promoter region of the GmKIX8-1 gene Glyma.17G112800,

optionally, (i) wherein said promoter region deletion comprises a deletion of the tandem repeated sequence CGC located at −177 to −174 relative to the GmKIX8-1 gene ATG start site;

optionally, (ii) wherein said promoter region deletion comprises a deletion of the tandem repeated sequence GT located at −104 to −92 relative to the GmKIX8-1 gene ATG start site; or optionally, wherein said promoter region deletion comprises both the deletions of (i) and (ii).

47. The method of identifying a soybean plant of any one of embodiments 40 to 46, wherein the genotype associated with the large-seed phenotype comprises at least one allele associated with the large-seed phenotype identified in the alignment of GmKIX8-1 genes in FIG. 17.
48. The method of identifying a soybean plant of embodiment 47, wherein the genotype associated with the large-seed phenotype comprises at least one of the two 3′ most deletions in the promoter region.
49. The method of identifying a soybean plant of embodiment 48, wherein the genotype associated with the large seed phenotype comprises at least the two 3′ most deletions in the promoter region.
50. The method of identifying a soybean plant of any one of embodiments 40 to 49, wherein the genotyping is done with a SSR marker primer pair comprising the sequences of F-KIX-SSR-GRIN GAGTGAGTGAGCACTGTGTTGTG (SEQ ID NO: 53) and R-KIX-SSR-GRIN ACCAAAACCGCCCCAAGACACTC (SEQ ID NO: 54), a primer pair comprising the sequences of F-KIX1CRISPGenotype TTCTCTCGCTACTCCTCCTACC (SEQ ID NO: 47) and R-KIX1CRISPGenotype GTACTCTGCCTAAGCAACAACCA (SEQ ID NO: 48), a primer pair comprising the sequences of F-KIX2CRISPGenotype GAGTGAGCGAGTGAGCACTGCC (SEQ ID NO: 49) and F-KIX2CRISPGenotype CAAATTCCGCAAGCATTTTGTG (SEQ ID NO: 50), or a primer pair comprising the sequences of F-KIX1-GRIN GGTACGGACATAGTTCACGATCCC (SEQ ID NO: 55) and R-KIX1-GRIN GATTCCTTGTCCATATCCATTATCC (SEQ ID NO: 56).
51. The method of identifying a soybean plant of any one of embodiments 40 to 50, wherein the genetic locus comprising the GmKIX8-1 gene Glyma.17G112800I having a genotype associated with a large-seed phenotype has been gene edited.
52. An edited soybean GmKIX8-1 gene comprising:

(i) a variant polynucleotide comprising a loss-of-function GmKIX8-1 gene variant, wherein the variant polynucleotide comprises at least one nucleotide insertion, deletion, and/or substitution in comparison to the corresponding unedited wild-type polynucleotide sequence, and wherein the variant polynucleotide exhibits reduced or loss of expression of the GmKIX8-1 gene in comparison to the wild-type polynucleotide sequence and/or encodes a GmKIX8-1 protein variant having reduced activity in comparison to wild-type GmKIX8-1 protein,

(ii) a variant polynucleotide encoding a loss-of-function GmKIX8-1 protein variant or fragment thereof,

wherein the variant polynucleotide comprises at least one nucleotide insertion, deletion, and/or substitution in comparison to the corresponding unedited wild-type polynucleotide sequence and does not encode for the wild-type GmKIX8-1 protein, and

wherein the variant polynucleotide encodes for a GmKIX8-1 protein variant having reduced activity in comparison to wild-type GmKIX8-1 protein,

optionally, wherein said variant polynucleotide is operably linked to a polynucleotide comprising a promoter;

(iii) a variant polynucleotide comprising a GmKIX8-1 gene 3′ UTR,

wherein the variant polynucleotide comprises at least one nucleotide insertion, deletion, and/or substitution in the 3′ UTR in comparison to the corresponding unedited wild-type polynucleotide sequence, and

wherein the variant 3′ UTR results in reduced or loss of expression of the GmKIX8-1 gene in comparison to the wild-type polynucleotide sequence,

optionally wherein said variant polynucleotide is operably linked to a polynucleotide comprising a promoter and/or a GmKIX8-1 protein coding region;

(iv) a variant polynucleotide comprising a GmKIX8-1 gene 5′ UTR, wherein the variant polynucleotide comprises at least one nucleotide insertion, deletion, and/or substitution in the 5′ UTR in comparison to the corresponding unedited wild-type polynucleotide sequence, and

wherein the variant 5′ UTR results in reduced or loss of expression of the GmKIX8-1 gene in comparison to the wild-type polynucleotide sequence,

optionally wherein said variant polynucleotide is operably linked to a polynucleotide comprising a promoter and/or a GmKIX8-1 protein coding region;

(v) a variant polynucleotide comprising a GmKIX8-1 gene promoter,

wherein the variant polynucleotide comprises at least one nucleotide insertion, deletion, and/or substitution in the promoter in comparison to the corresponding unedited wild-type polynucleotide sequence, and

wherein the variant promoter results in reduced or loss of expression of the GmKIX8-1 gene in comparison to the wild-type polynucleotide sequence,

optionally, wherein said variant polynucleotide is operably linked to a polynucleotide comprising a GmKIX8-1 protein coding region;

(vi) a variant polynucleotide comprising a GmKIX8-1 gene intron,

wherein the variant polynucleotide comprises at least one nucleotide insertion, deletion, and/or substitution in the intron in comparison to the corresponding unedited wild-type polynucleotide sequence, and

wherein the variant intron results in reduced or loss of expression of the GmKIX8-1 gene in comparison to the wild-type polynucleotide sequence, optionally, wherein said variant polynucleotide is operably linked to a polynucleotide comprising at least one GmKIX8-1 gene exon; and/or

(vii) a variant polynucleotide comprising a GmKIX8-1 gene exon,

wherein the variant polypeptide comprises at least one nucleotide insertion, deletion, and/or substitution in the exon in comparison to the corresponding unedited wild-type polynucleotide sequence, and

wherein the variant exon results in reduced or loss of expression of the GmKIX8-1 gene in comparison to the wild-type polynucleotide sequence and/or encodes a GmKIX8-1 protein variant having reduced activity in comparison to wild-type GmKIX8-1 protein,

optionally wherein said variant polynucleotide is operably linked to a polynucleotide comprising a promoter and/or a GmKIX8-1 gene intron, and

optionally, wherein the variant polypeptide comprises a deletion of at least a portion of exon 1, exon 2, or both exon 1 and exon 2 of the GmKIX8-1 gene.

53. The edited soybean GmKIX8-1 gene of embodiment 52 comprising a variant polypeptide comprising a loss-of-function GmKIX8-1 gene variant,

wherein the variant polynucleotide comprises a deletion of a portion of the 3′ end of exon 1 of the GmKIX8-1 gene, a deletion of the intron between exon 1 and exon 2 of the GmKIX8-1 gene, and a deletion of a portion the 5′ end of exon 2 of the GmKIX8-1 gene in comparison to the corresponding unedited wild-type polynucleotide sequence.

54. The edited soybean GmKIX8-1 gene of embodiment 52 wherein the variant polynucleotide comprises at least one nucleotide deletion in the promoter in comparison to the corresponding unedited wild-type polynucleotide sequence, and

wherein the nucleotide deletion results in reduced or loss of expression of the GmKIX8-1 gene in comparison to the corresponding wild-type polynucleotide sequence.

55. The edited soybean GmKIX8-1 gene of embodiment 52 comprising a variant polynucleotide encoding a loss-of-function GmKIX8-1 protein variant or fragment thereof,

wherein the variant polynucleotide comprises at least one nucleotide insertion, deletion, and/or substitution in comparison to the corresponding unedited wild-type polynucleotide sequence and does not encode for the wild-type GmKIX8-1 protein, and

wherein the variant polynucleotide encodes for a GmKIX8-1 protein variant having reduced activity in comparison to wild-type GmKIX8-1 protein.

56. The edited soybean GmKIX8-1 gene of embodiment 55, wherein nucleotide insertion, deletion, and/or substitution results in a frameshift mutation and/or a nonsense mutation.
57. A plant nuclear genome comprising the edited soybean GmKIX8-1 gene of any one of embodiments 52-56;

optionally, wherein the variant polynucleotide is heterologous to the nuclear genome.

58. The plant nuclear genome of embodiment 57, wherein said variant polynucleotide is operably linked to an endogenous promoter of the nuclear genome.
59. The edited gene or the nuclear genome of any one of embodiments 52-58, further comprising a polynucleotide encoding (i) a transit peptide, a vacuolar targeting peptide, and/or an endoplasmic reticulum targeting peptide; (ii) a plastid targeting peptide; and/or (iii) a polyadenylation or transcriptional termination signal, wherein the polynucleotides of (i), (ii), and/or (iii) are operably linked to the polypeptide encoding the soybean GmKIX8-1 protein.
60. A cell comprising the edited gene or nuclear genome of any one of embodiments 52-59.
61. The cell of embodiment 60, wherein the cell is a plant, yeast, mammalian, or bacterial cell.
62. The cell of embodiment 60, wherein the cell is a plant cell that is non-regenerable.
63. A soybean plant comprising the edited soybean GmKIX8-1 gene or nuclear genome of any one of embodiments 52-59
64. The soybean plant of embodiment 63, wherein the edited soybean GmKIX8-1 gene or nuclear genome confers to the plant larger seed size in comparison to a control plant that lacks the edited gene or nuclear genome;

optionally, wherein the larger seed size in comparison to a control plant that lacks the edited gene or nuclear genome is at least about 3%, 5%, 10%, 15%, 20%, 25%, or 30% higher 100-seed weight,

optionally, wherein the edited soybean GmKIX8-1 gene or nuclear genome confers to the plant a large-seed phenotype.

65. A plant part of the soybean plant of embodiment 64, wherein the plant part comprises the edited soybean GmKIX8-1 gene or nuclear genome.
66. The plant part of embodiment 65, wherein the plant part is a seed, stem, leaf, root, or flower;

optionally, where the plant part is a seed.

67. A method for producing a soybean plant comprising the edited soybean GmKIX8-1 gene or plant genome of any of embodiments 52-59,

wherein edited soybean GmKIX8-1 gene exhibits reduced or loss of expression of the GmKIX8-1 gene in comparison to the wild-type polynucleotide sequence and/or encodes a GmKIX8-1 protein variant having reduced activity in comparison to wild-type GmKIX8-1 protein,

the method comprising introducing into a plant cell one or more gene-editing molecules that target an endogenous soybean GmKIX8-1 gene to introduce at least one nucleotide insertion, deletion, and/or substitution into the endogenous GmKIX8-1 gene.

68. The method for producing a soybean plant of embodiment 67, wherein the method comprises a gene-editing method selected from the group consisting of zinc finger nucleases, transcription activator-like effector nucleases (TALENs), engineered homing endonucleases/meganucleases, and the clustered regularly interspaced short palindromic repeat (CRISPR)-associated protein9 (Cas9) system.
69. The method for producing a soybean plant of embodiment 67 or 68, wherein the soybean plant obtained exhibits a large-seed phenotype.
70. The method for producing a soybean plant of any one of embodiments 67 to 69, comprising the steps of:

(i) providing to a plant cell, tissue, part, or whole plant an endonuclease or an endonuclease and at least one guide RNA, wherein the endonuclease or guide RNA and endonuclease can form a complex that can introduce a double-strand break at a target site in a genome of the plant cell, tissue, part, or whole plant;

(ii) obtaining a plant cell, tissue, part, or whole plant wherein at least one nucleotide insertion, deletion, and/or substitution has been introduced into the corresponding wild-type polynucleotide sequence; and

(iii) selecting a plant obtained from the plant cell, tissue, part or whole plant of step (ii) comprising the edited soybean GmKIX8-1 gene.

72. The method for producing a soybean plant of embodiment 71, wherein the endonuclease is a Cas endonuclease;

optionally, wherein the guide RNA is a clustered regularly interspaced short palindromic repeats (CRISPR)-associated (Cas)-guide RNA,

optionally, wherein the guide RNA comprises SEQ ID NO: 87 and/or SEQ ID NO: 88.

73. The method for producing a soybean plant of embodiment 70 or 71, wherein the selected soybean plant exhibits a large-seed phenotype.
74. A gene-edited soybean plant having a large-seed phenotype, wherein the soybean plant comprises a variant polynucleotide comprising a targeted loss-of-function GmKIX8-1 gene variant which comprises an insertion, substitution, and/or a deletion in the GmKIX8-1 gene Glyma.17G112800 that reduces expression of the GmKIX8-1 gene Glyma.17G112800 compared to wild-type expression and/or encodes a GmKIX8-1 protein variant having reduced activity in comparison to wild-type GmKIX8-1 protein.
75. A seed produced by a soybean plant of any one of embodiments 29-39, 63, 64, or 74, wherein the seed comprises a detectable amount of the genetic locus that comprises a genotype associated with a large-seed phenotype;

optionally, wherein the seed comprises an endogenous edited soybean GmKIX8-1 gene comprising the variant polynucleotide.

76. The seed of embodiment 75, wherein the seed is coated with a composition comprising an insecticide and/or a fungicide.
77. A plant propagation material comprising the coated seed of embodiment 76.
78. A method of producing a commercial crop seed lot of soybean seeds comprising in their genomes at least one introgressed large-seed phenotype genetic locus, the method comprising the steps of:

a. producing a population of soybean plants from the soybean plant selected in step (c) of any one of embodiments 17 to 28 comprising a genotype associated with a large-seed phenotype and at least one linked marker found in said second soybean plant comprising a non-large seed phenotype genetic locus but not found in said first soybean plant; and

b. harvesting a commercial seed lot, wherein the harvested crop seed lot comprises a plurality of seeds that comprise in their genomes at least one introgressed large-seed phenotype genetic locus.

79. The method of producing a commercial crop seed lot of embodiment 78, wherein the seed lot comprise at least 100 seeds, at least 500 seeds, at least 1,000 seeds, at least 5,000 seeds, at least 10,000 seeds, at least 25,000 seeds, at least 50,000 seeds, or at least 100,000 seeds.
80. The method of producing a commercial crop seed lot of embodiment 78 or 79, wherein the plurality of seeds that comprise in their genomes at least one introgressed genetic locus comprising the GmKIX8-1 gene Glyma.17G112800I having a genotype associated with a large-seed phenotype constitute at least 85%, at least 90%, at least 91%, at least 92%, at least 93%, at least 94%, at least 95%, at least 96%, at least 97%, at least 98%, or at least 99% of the seed of the harvested seed lot.
81. The method of producing a commercial crop seed lot of any one of embodiments 78 to
80, wherein the method further comprises the step of packaging seed obtained from said seed lot into one or more bags to obtain one or more packaged seed bags.
82. The method of producing a commercial crop seed lot of any one of embodiments 78 to
81, wherein said method further comprises the step of distributing the packaged seeds to growers for use in crop production.
83. The method of producing a commercial crop seed lot of any one of embodiments 78 to
82, wherein the genetic locus comprising the GmKIX8-1 gene Glyma.17G112800I having a genotype associated with a large-seed phenotype has been gene edited.
84. A commercial crop seed lot of soybean seeds comprising a plurality of seeds that comprise in their genomes at least one introgressed genetic locus comprising the soybean GmKIX8-1 gene Glyma.17G112800I having a genotype associated with a large-seed phenotype.
85. The commercial crop seed lot of embodiment 84, wherein the seed lot comprise at least 100 seeds, at least 500 seeds, at least 1,000 seeds, at least 5,000 seeds, at least 10,000 seeds, at least 25,000 seeds, at least 50,000 seeds, or at least 100,000 seeds.
86. The commercial crop seed lot of embodiment 84 or 85, wherein the plurality of seeds that comprise in their genomes at least one introgressed genetic locus comprising the soybean GmKIX8-1 gene Glyma.17G112800I having a genotype associated with a large-seed phenotype constitute at least 85%, at least 90%, at least 91%, at least 92%, at least 93%, at least 94%, at least 95%, at least 96%, at least 97%, at least 98%, or at least 99% of the seed of the seed lot.
87. The commercial crop seed lot of any one of embodiments 84 to 86, made by the method of producing a commercial crop seed lot of any one of embodiments 78 to 83.
88. A method of increasing soybean seed weight, the method comprising reducing or abolishing expression of the GmKIX8-1 gene and/or reducing or abolishing activity of the GmKIX9-1 protein.
89. The method of increasing soybean seed weight of claim 88, wherein the reduction in expression and/or activity is accomplished by a gene editing technology.
90. The method of increasing soybean seed weight of claim 88, wherein the reduction in expression and/or activity is accomplished by a gene knockdown technology, optionally selected from antisense oligonucleotides, RNAi, and miRNA.

GmKIX8-1 promoter sequences used in dual LUC assays. DNA Sequences for from Williams 82, PI597483 and the three promoter deletion versions (Del1, Del2, and Del3) are shown. 1.6 kb promoter region of:

>1.6 kb_W82
(SEQ ID NO: 67)
GGTACGGACATAGTTCACGATCCCTTCCACCATATCTGTTTTAATTTTATTTATTATTT
TTGAAGTTTGTCTTGTAATATAAATTGAAATGTGTTGTACTTTGGAGTTTAAGGAGCA
TCAAGTATCAAGTAGAAACATGGAAGGGTGAGGAGGAGATTATTCACCCACTGTTG
AAACATTAGGCtttttttttttttttttactttttCCAAGTACTGTACCCCCCCCCCCCAAAGGAAAAAA
GATAGAAGTTGGAAGTGGGATGCTCGAGGCTTACATAAAAACTGAAAAAGGAGTTT
GGCTTTGTGCAGCCGTTGTCAAGAGTGGACGTGACATGAGAGAGTGGGGAAGGCCC
ATACAGCGTCAGTGTTATGCCAAAGGCTGTTTTCATCACATAAAGGGAATTGTTTAC
AGCGCCGAGGGTGCAGAATGTACCCGTTTACCCATCAATGTGTTCACTGCACATTCA
ATGCACaatataaatatatatatcttcataagtcataaataAATCAATTTTACTTACTAGACACTAGACACG
GGTCAAGGTGACTCAGATAACGTAGTCATAGACATTAATTTCTACAAGGGCTGACCC
ACCAACATAACAATTGAGAGAGCACCATGTGCACGTTGTAGGTGGCAACAAGAGCC
TAGCACAACCGAAATAACGATGTTTGTCCCCGGAATAAAAGCATGAGTCACGGTGA
AACAAAAATTAACCCTAAGGGTAAAATAAAAAAATAAAAAAAGGAGAAAGAATCT
GAGTAGCTTTATTAAGGAAGAATAAGGGTGGGAGGGAAACTGATGTAACCGGATTC
TCTTGGGCTAACTTTATAACGAAGTTGATATTAGTTATGTTTTGTCATTTTCCTTGGC
AGTGTTCATGCTGGTTGAAGGGTTATGTGTACGAATATGCTGGCATTGGAGATGGAT
GCGCAATTatgtaaaatagataataatgcattgcatggtaatgtaatgttatgtaaCAACAACAACCAACCCTATAT
TAAATATGGGTGAGGGCATGGCATTGGGAAGGGGAGAATTTTAATGGGACTTTATTA
TACAGGTACTTTTTGATAGTAACCTAACTCTCCCACTCTCCTTTTCTCAAGAGGTGTG
ACCAGACCCCACCCCCCTACTCACACAAAGGAGAAAAGATTGAACACTAACATCAA
ACAACAAACAAAAAAGACCATGGTAATGGTAAACCCTCTAAATTATTGGGGAAGAC
AAAAGAATGAGAAAGAAAGCTGCCCTTTCTCTCGCTACTCCTCCTACCCCAAACAAA
ACTTTCCCATTACAAATTGTGGAATGAAGAAAGAATGGCAAGACAAGTGAGTAGTA
GTATAGTAGTAATTGACCCATCAAACAAACAAGCATAACCCATGTAATTGTTATTGT
AGATAGATAGAGTGAGTGAGTGAGCACTGTGTTGTGTATATAGTAGTGTGTAAACTC
TCCCCAACAGACAGACACGCCGCCACTCAATTATTTCTGTCCATTTCTTCTTCCCATA
TTGTTCTCGTGTGTTGTGTGTGTCAgtgttttggtgtgtgtgtgtgtgtgtgtgtgagtgagagaaagagaaaaaaaga
aaAGAAAAAGAGTGTCTTGGGGCGGTTTTGGTTTGAGTTTAACACTTTCATTTCGAAG
>1.6 Kb_PI597483
(SEQ ID NO: 68)
GGTACGGACATAGTTCACGATCCCTTCCACCATATCTGTTTTAATTTTATTTATTATTT
TTGAAGTTTGTCTTGTAATATAAATTGAAATGTGTTGTACTTTGGAGTTTAAGGAGCA
TCAAGTATCAAGTAGAAACATGGAAGGGTGAGGAGGAGATTATTCACCCACTGTTG
AAACATTAGGCtttttttttttttttttactttttCCAAGTACTGTACCCCCCCCCCCCCAAAGGAAAAAA
GATAGAAGTTGGAAGTGGGATGCTCGAGGCTTACATAAAAACTGAAAAAGGAGTTT
GGCTTTGTGCAGCCGTTGTCAAGAGTGGACGTGACATGAGAGAGTGGGGAAGGCCC
ATACAGCGTCAGTGTTATGCCAAAGGCTGTTTTCATCACATAAAGGGAATTGTTTAC
AGCGCCGAGGGTGCAGAATGTACCCGTTTACCCATCAATGTGTTCACTGCACATTCA
ATGCACaatataaatatatatatcttcataagtcataaataAATCAATTTTACTTACTAGACACTAGACACG
GGTCAAGGTGACTCAGATAACGTAGTCATAGACATTAATTTCTACAAGGGCTGACCC
ACCAACATAACAATTGAGAGAGCACCATGTGCACGTTGTAGGTGGCAACAAGAGCC
TAGCACAACCGAAATAACGATGTTTGTCCCCGGAATAAAAGCATGAGTCACGGTGA
AACAAAAATTAACCCTAAGGGTAAAATAAAAAAATAAAAAAAGGAGAAAGAATCT
GAGTAGCTTTATTAAGGAAGAATAAGGGTGGGAGGGAAACTGATGTAACCGGATTC
TCTTGGGCTAACTTTATAACGAAGTTGATATTAGTTATGTTTTGTCATTTTCCTTGGC
AGTGTTCATGCTGGTTGAAGGGTTATGTGTACGAATATGCTGGCATTGGAGATGGAT
GCGCAATTatgtaaaatagataataatgcattgcatggtaatgtaatgttatgtaaCAACAACAACCAACCCTATAT
TAAATATGGGTGAGGGCATGGCATTGGGAAGGGGAGAATTTTAATGGGACTTTATTA
TACAGGTACTTTTTGATAGTAACCTAACTCTCCCACTCTCCTTTTCTCAAGAGGTGTG
ACCAGACCCCACCCCCCTACTCACACAAAGGAGAAAAGATTGAACACTAACATCAA
ACAACAAACAAAAAAGACCATGGTAATGGTAAACCCTCTAAATTATTGGGGAAGAC
AAAAGAATGAGAAAGAAAGCTGCCCTTTCTCTCGCTACTCCTCCTACCCCAAACAAA
ACTTTCCCATTACAAATTGTGGAATGAAGAAAGAATGGCAAGACAAGTGAGTAGTA
GTATAGTAGTAATTGACCCATCAAACAAACAAGCATAACCCATGTAATTGTTATTGT
AGATAGATAGAGTGAGTGAGTGAGCACTGTGTTGTGTATATAGTAGTGTGTAAACTC
TCCCCAACAGACAGACACGCCACTCAATTATTTCTGTCCATTTCTTCTTCCCATATTG
TTCTCGTGTGTTGTGTGTGTCAgtgttttggtgtgtgtgtgagtgagagaaagagaaaaaaagaaaAGAAAAAG
AGTGTCTTGGGGCGGTTTTGGTTTGAGTTTAACACTTTCATTTCGAAG
>del1
(SEQ ID NO: 69)
GGTACGGACATAGTTCACGATCCCTTCCACCATATCTGTTTTAATTTTATTTATTATTT
TTGAAGTTTGTCTTGTAATATAAATTGAAATGTGTTGTACTTTGGAGTTTAAGGAGCA
TCAAGTATCAAGTAGAAACATGGAAGGGTGAGGAGGAGATTATTCACCCACTGTTG
AAACATTAGGCtttttttttttttttttctttttCCAAGTACTGTACCCCCCCCCCCCCAAAGGAAAAAA
GATAGAAGTTGGAAGTGGGATGCTCGAGGCTTACATAAAAACTGAAAAAGGAGTTT
GGCTTTGTGCAGCCGTTGTCAAGAGTGGACGTGACATGAGAGAGTGGGGAAGGCCC
ATACAGCGTCAGTGTTATGCCAAAGGCTGTTTTCATCACATAAAGGGAATTGTTTAC
AGCGCCGAGGGTGCAGAATGTACCCGTTTACCCATCAATGTGTTCACTGCACATTCA
ATGCACaatataaatatatatatcttcataagtcataaataAATCAATTTTACTTACTAGACACTAGACACG
GGTCAAGGTGACTCAGATAACGTAGTCATAGACATTAATTTCTACAAGGGCTGACCC
ACCAACATAACAATTGAGAGAGCACCATGTGCACGTTGTAGGTGGCAACAAGAGCC
TAGCACAACCGAAATAACGATGTTTGTCCCCGGAATAAAAGCATGAGTCACGGTGA
AACAAAAATTAACCCTAAGGGTAAAATAAAAAAATAAAAAAAGGAGAAAGAATCT
GAGTAGCTTTATTAAGGAAGAATAAGGGTGGGAGGGAAACTGATGTAACCGGATTC
TCTTGGGCTAACTTTATAACGAAGTTGATATTAGTTATGTTTTGTCATTTTCCTTGGC
AGTGTTCATGCTGGTTGAAGGGTTATGTGTACGAATATGCTGGCATTGGAGATGGAT
GCGCAATTatgtaaaatagataataatgcattgcatggtaatgtaatgttatgtaaCAACAACAACCAACCCTATAT
TAAATATGGGTGAGGGCATGGCATTGGGAAGGGGAGAATTTTAATGGGACTTTATTA
TACAGGTACTTTTTGATAGTAACCTAACTCTCCCACTCTCCTTTTCTCAAGAGGTGTG
ACCAGACCCCACCCCCCTACTCACACAAAGGAGAAAAGATTGAACACTAACATCAA
ACAACAAACAAAAAAGACCATGGTAATGGTAAACCCTCTAAATTATTGGGGAAGAC
AAAAGAATGAGAAAGAAAGCTGCCCTTTCTCTCGCTACTCCTCCTACCCCAAACAAA
ACTTTCCCATTACAAATTGTGGAATGAAGAAAGAATGGCAAGACAAGTGAGTAGTA
GTATAGTAGTAATTGACCCATCAAACAAACAAGCATAACCCATGTAATTGTTATTGT
AGATAGATAGAGTGAGTGAGTGAGCACTGTGTTGTGTATATAGTAGTGTGTAAACTC
TCCCCAACAGACAGACACACTCAATTATTTCTGTCCATTTCTTCTTCCCATATTGTTCT
CGTGTGTTGTGTGTGTCAgtgttttggtgtgtgtgtgtgtgtgtgtgtgagtgagagaaagagaaaaaaagaaaAGAAA
AAGAGTGTCTTGGGGCGGTTTTGGTTTGAGTTTAACACTTTCATTTCGAAG
>del2
(SEQ ID NO: 70)
GGTACGGACATAGTTCACGATCCCTTCCACCATATCTGTTTTAATTTTATTTATTATTT
TTGAAGTTTGTCTTGTAATATAAATTGAAATGTGTTGTACTTTGGAGTTTAAGGAGCA
TCAAGTATCAAGTAGAAACATGGAAGGGTGAGGAGGAGATTATTCACCCACTGTTG
AAACATTAGGCtttttttttttttttttactttttCCAAGTACTGTACCCCCCCCCCCCCAAAGGAAAAAA
GATAGAAGTTGGAAGTGGGATGCTCGAGGCTTACATAAAAACTGAAAAAGGAGTTT
GGCTTTGTGCAGCCGTTGTCAAGAGTGGACGTGACATGAGAGAGTGGGGAAGGCCC
ATACAGCGTCAGTGTTATGCCAAAGGCTGTTTTCATCACATAAAGGGAATTGTTTAC
AGCGCCGAGGGTGCAGAATGTACCCGTTTACCCATCAATGTGTTCACTGCACATTCA
ATGCACaatataaatatatatatcttcataagtcataaataAATCAATTTTACTTACTAGACACTAGACACG
GGTCAAGGTGACTCAGATAACGTAGTCATAGACATTAATTTCTACAAGGGCTGACCC
ACCAACATAACAATTGAGAGAGCACCATGTGCACGTTGTAGGTGGCAACAAGAGCC
TAGCACAACCGAAATAACGATGTTTGTCCCCGGAATAAAAGCATGAGTCACGGTGA
AACAAAAATTAACCCTAAGGGTAAAATAAAAAAATAAAAAAAGGAGAAAGAATCT
GAGTAGCTTTATTAAGGAAGAATAAGGGTGGGAGGGAAACTGATGTAACCGGATTC
TCTTGGGCTAACTTTATAACGAAGTTGATATTAGTTATGTTTTGTCATTTTCCTTGGC
AGTGTTCATGCTGGTTGAAGGGTTATGTGTACGAATATGCTGGCATTGGAGATGGAT
GCGCAATTatgtaaaatagataataatgcattgcatggtaatgtaatgttatgtaaCAACAACAACCAACCCTATAT
TAAATATGGGTGAGGGCATGGCATTGGGAAGGGGAGAATTTTAATGGGACTTTATTA
TACAGGTACTTTTTGATAGTAACCTAACTCTCCCACTCTCCTTTTCTCAAGAGGTGTG
ACCAGACCCCACCCCCCTACTCACACAAAGGAGAAAAGATTGAACACTAACATCAA
ACAACAAACAAAAAAGACCATGGTAATGGTAAACCCTCTAAATTATTGGGGAAGAC
AAAAGAATGAGAAAGAAAGCTGCCCTTTCTCTCGCTACTCCTCCTACCCCAAACAAA
ACTTTCCCATTACAAATTGTGGAATGAAGAAAGAATGGCAAGACAAGTGAGTAGTA
GTATAGTAGTAATTGACCCATCAAACAAACAAGCATAACCCATGTAATTGTTATTGT
AGATAGATAGAGTGAGTGAGTGAGCACTGTGTTGTGTATATAGTAGTGTGTAAACTC
TCCCCAACAGACAGACACGCCGCCACTCAATTATTTCTGTCCATTTCTTCTTCCCATA
TTGTTCTCCTTGGGGCGGTTTTGGTTTGAGTTTAACACTTTCATTTCGAAG
>del3
(SEQ ID NO: 71)
GGTACGGACATAGTTCACGATCCCTTCCACCATATCTGTTTTAATTTTATTTATTATTT
TTGAAGTTTGTCTTGTAATATAAATTGAAATGTGTTGTACTTTGGAGTTTAAGGAGCA
TCAAGTATCAAGTAGAAACATGGAAGGGTGAGGAGGAGATTATTCACCCACTGTTG
AAACATTAGGCtttttttttttttttttactttttCCAAGTACTGTACCCCCCCCCCCCCAAAGGAAAAAA
GATAGAAGTTGGAAGTGGGATGCTCGAGGCTTACATAAAAACTGAAAAAGGAGTTT
GGCTTTGTGCAGCCGTTGTCAAGAGTGGACGTGACATGAGAGAGTGGGGAAGGCCC
ATACAGCGTCAGTGTTATGCCAAAGGCTGTTTTCATCACATAAAGGGAATTGTTTAC
AGCGCCGAGGGTGCAGAATGTACCCGTTTACCCATCAATGTGTTCACTGCACATTCA
ATGCACaatataaatatatatatcttcataagtcataaataAATCAATTTTACTTACTAGACACTAGACACG
GGTCAAGGTGACTCAGATAACGTAGTCATAGACATTAATTTCTACAAGGGCTGACCC
ACCAACATAACAATTGAGAGAGCACCATGTGCACGTTGTAGGTGGCAACAAGAGCC
TAGCACAACCGAAATAACGATGTTTGTCCCCGGAATAAAAGCATGAGTCACGGTGA
AACAAAAATTAACCCTAAGGGTAAAATAAAAAAATAAAAAAAGGAGAAAGAATCT
GAGTAGCTTTATTAAGGAAGAATAAGGGTGGGAGGGAAACTGATGTAACCGGATTC
TCTTGGGCTAACTTTATAACGAAGTTGATATTAGTTATGTTTTGTCATTTTCCTTGGC
AGTGTTCATGCTGGTTGAAGGGTTATGTGTACGAATATGCTGGCATTGGAGATGGAT
GCGCAATTatgtaaaatagataataatgcattgcatggtaatgtaatgttatgtaaCAACAACAACCAACCCTATAT
TAAATATGGGTGAGGGCATGGCATTGGGAAGGGGAGAATTTTAATGGGACTTTATTA
TACAGGTACTTTTTGATAGTAACCTAACTCTCCCACTCTCCTTTTCTCAAGAGGTGTG
ACCAGACCCCACCCCCCTACTCACACAAAGGAGAAAAGATTGAACACTAACATCAA
ACAACAAACAAAAAAGACCATGGTAATGGTAAACCCTCTAAATTATTGGGGAAGAC
AAAAGAATGAGAAAGAAAGCTGCCCTTTCTCTCGCTACTCCTCCTACCCCAAACAAA
ACTTTCCCATTACAAATTGTGGAATGAAGAAAGAATGGCAAGACAAGTGAGTAGTA
GTATAGTAGTAATTGACCCATCAAACAAACAAGCATAACCCATGTAATTGTTATTGT
AGATAGATAGAGTGAGTGAGTGAGCACTGTGTTGTGTATATAGTAGTGTGTAAACTC
TCCCCAACAGACAGACACTTGGGGCGGTTTTGGTTTGAGTTTAACACTTTCATTTCGA
AG

### REFERENCES

https://soybase.org/
www.ars-grin.gov
http://rsb.info.nih.gov/ij
https://phytozome.jgi.doe.gov
Adamski N M, Anastasiou E, Eriksson S, O'Neill C M, Lenhard M. 2009. Local maternal control of seed size by KLUH/CYP78A5 dependent growth signaling. Proceedings of the National Academy of Sciences, USA 106: 20115.
Baekelandt A, Pauwels L, Wang Z, Li N, De Milde L, Natran A, Vermeersch M, Li Y, Goossens A, Inz'eD et al. 2018. Arabidopsis leaf flatness is regulated by ppd2 and ninja through repression of CYCLIN D3 genes. Plant Physiology 178: 217.
Bailey T L, Boden M, Buske F A, Frith M, Grant C E, Clementi L, Ren J, Li W W, Noble W S. 2009. MEME SUITE: tools for motif discovery and searching. Nucleic Acids Research 37: W202-W208.
Barbeira A N, Dickinson S P, Bonazzola R, Zheng J, Wheeler H E, Torres J M, Torstenson E S, Shah K P, Garcia T, Edwards T L et al. 2018. Exploring the phenotypic consequences of tissue specific gene expression variation inferred from GWAS summary statistics. Nature Communications 9: 1825.
Bashandy H, Jalkanen S, Teen T H. 2015. Within leaf variation is the largest source of variation in agroinfiltration of Nicotiana benthamiana. Plant Methods 11: 47.
Birchler J A, Bhadra U, Bhadra M P, Auger D L. 2001. Dosage-dependent gene regulation in multicellular eukaryotes: implications for dosage compensation, aneuploid syndromes, and quantitative traits. Developmental Biology 234: 275-288.
Boell L, Pallares L F, Brodski C, Chen Y, Christian J L, Kousa Y A, Kuss P, Nelsen S, Novikov O, Schutte B C et al. 2013. Exploring the effects of gene dosage on mandible shape in mice as a model for studying the genetic basis of natural variation. Development Genes and Evolution 223: 279-287.
Bolon Y-T, Haun W J, Xu W W, Grant D, Stacey M G, Nelson R T, Gerhardt D J, Jeddeloh J A, Stacey G, Muehlbauer G J et al. 2011. Phenotypic and genomic analyses of a fast neutron mutant population resource in soybean. Plant Physiology 156: 240.
Burris J S, Edje O T, Wahab A H. 1973. Effects of seed size on seedling performance in soybeans: II. Seedling growth and photosynthesis and field performance. Crop Science 13: cropsci1973.0011183X001300020017x.
Campbell B W, Stupar R M. 2016. Soybean (Glycine max) mutant and germplasm resources: current status and future prospects. Current Protocols in Plant Biology 1: 307-327.
Curtis M D, Grossniklaus U. 2003. A gateway cloning vector set for high-throughput functional analysis of genes in planta. Plant Physiology 133: 462.
Dilkes B P, Comai L. 2004. A differential dosage hypothesis for parental effects in seed development. The Plant Cell 16: 3174.
Disch S, Anastasiou E, Sharma V K, Laux T, Fletcher J C, Lenhard M. 2006. The E3 ubiquitin ligase BIG BROTHER controls Arabidopsis organ size in a dosage-dependent manner. Current Biology 16: 272-279.
Do P T, Nguyen C X, Bui H T, Tran LTN, Stacey G, Gillman J D, Zhang Z J, Stacey M G. 2019. Demonstration of highly efficient dual gRNA CRISPR/Cas9 editing of the homeologous GmFAD2-1A and GmFAD2-1B genes to yield a high oleic, low linoleic and a-linolenic acid phenotype in soybean. BMC Plant Biology 19: 311.
Dobbels A A, Michno J-M, Campbell B W, Virdi K S, Stec A O, Muehlbauer G J, Naeve S L, Stupar R M. 2017. An induced chromosomal translocation in soybean disrupts a KASI ortholog and is associated with a high sucrose and low oil seed phenotype. G3: Genes|Genomes|Genetics 7:1215-1223.
Edwards C J, Hartwig E E. 1971. Effect of seed size upon rate of germination in soybeans. Agronomy Journal 63: 429-450.
Ge L, Yu J, Wang H, Luth D, Bai G, Wang K, Chen R. 2016. Increasing seed size and quality by manipulating BIG SEEDS1 in legume species. Proceedings of the National Academy of Sciences, USA 113: 12414.
Gillman J D, Stacey M G, Cui Y, Berg H R, Stacey G. 2014. Deletions of the SACPD-C locus elevate seed stearic acid levels but also result in fatty acid and morphological alterations in nitrogen fixing nodules. BMC Plant Biology 14: 143.
Gonzalez N, Pauwels L, Baekelandt A, De Milde L, Van Leene J, Besbrugge N, Heyndrickx K S, P'erez A C, Durand A N, De Clercq R et al. 2015. A repressor protein complex regulates leaf growth in Arabidopsis. Plant Cell 27: 2273.
Grant D, Nelson R T, Cannon S B, Shoemaker R C. 2009. SoyBase, the USDA-ARS soybean genetics and genomics database. Nucleic Acids Research 38: D843-D846.
Haun W J, Hyten D L, Xu W W, Gerhardt D J, Albert T J, Richmond T, Jeddeloh J A, Jia G, Springer N M, Vance C P et al. 2011. The composition and origins of genomic variation among individuals of the soybean reference cultivar Williams 82. Plant Physiology 155: 645.
Hellemans J, Mortier G, De Paepe A, Speleman F, Vandesompele J. 2007. qBase relative quantification framework and software for management and automated analysis of real-time quantitative PCR data. Genome Biology 8: R19.
Hellens R P, Allan A C, Friel E N, Bolitho K, Grafton K, Templeton M D, Karunairetnam S, Gleave A P, Laing W A. 2005. Transient expression vectors for functional genomics, quantification of promoter activity and RNA silencing in plants. Plant Methods 1: 13.
Hepworth J, Lenhard M. 2014. Regulation of plant lateral-organ growth by modulating cell number and size. Growth and development 17: 36-42.
Hoeck J A, Fehr W R, Shoemaker R C, Welke G A, Johnson S L, Cianzio S R. 2003. Molecular marker analysis of seed size in soybean. Crop Science 43: 68-74.
Hopper N W, Overholt J R, Martin J R. 1979. Effect of cultivar, temperature and seed size on the germination and emergence of soya beans (Glycine max (L.) Merr.). Annals of Botany 44: 301-308.
Horiguchi G, Ferjani A, Fujikura U, Tsukaya H. 2006. Coordination of cell proliferation and cell expansion in the control of leaf size in Arabidopsis thaliana. Journal of Plant Research 119: 37-42.
Horiguchi G, Kim G-T, Tsukaya H. 2005. The transcription factor AtGRF5 and the transcription coactivator AN3 regulate cell proliferation in leaf primordia of Arabidopsis thaliana. The Plant Journal 43: 68-78.
Hwang W J, Kim M Y, Kang Y J, Shim S, Stacey M G, Stacey G, Lee S-H. 2015. Genome-wide analysis of mutations in a dwarf soybean mutant induced by fast neutron bombardment. Euphytica 203: 399-408.
Hyten D L, Song Q, Zhu Y, Choi I-Y, Nelson R L, Costa J M, Specht J E, Shoemaker R C, Cregan P B. 2006. Impacts of genetic bottlenecks on soybean genome diversity. Proceedings of the National Academy of Sciences, USA 103: 16666.
Jiang W-B, Huang H-Y, Hu Y-W, Zhu S-W, Wang Z-Y, Lin W-H. 2013. Brassinosteroid regulates seed size and shape in arabidopsis. Plant Physiology 162: 1965-1977
Jing Y, Zhao X, Wang J, Teng W, Qiu L, Han Y, Li W. 2018. Identification of the genomic region underlying seed weight per plant in soybean (Glycine max L. Merr.) via high-throughput single-nucleotide polymorphisms and a genome-wide association study. Frontiers Plant Science 9: 1392.
Kanazashi Y, Hirose A, Takahashi I, Mikami M, Endo M, Hirose S, Toki S, Kaga A, Naito K, Ishimoto M et al. 2018. Simultaneous site-directed mutagenesis of duplicated loci in soybean using a single guide RNA. Plant Cell Reports 37: 553-563.
Kaplan-Levy R N, Brewer P B, Quon T, Smyth D R. 2012. The trihelix family of transcription factors—light, stress and development. Trends in Plant Science 17: 163-171.
Karikari B, Chen S, Xiao Y, Chang F, Zhou Y, Kong J, Bhat J A, Zhao T. 2019. Utilization of interspecific high-density genetic map of RIL population for the QTL detection and candidate gene mining for 100-seed weight in soybean. Frontiers in Plant Science 10: 1001.
Kato S, Sayama T, Fujii K, Yumoto S, Kono Y, Hwang T-Y, Kikuchi A, Takada Y, Tanaka Y, Shiraiwa T et al. 2014. A major and stable QTL associated with seed weight in soybean across multiple environments and genetic backgrounds. Theoretical and Applied Genetics 127: 1365-1374.
Kim H-K, Kim Y-C, Kim S-T, Son B-G, Choi Y-W, Kang J-S, Park Y-H, Cho Y-S, Choi I-S. 2010. Analysis of quantitative trait loci (QTLs) for seed size and fatty acid composition using recombinant inbred lines in soybean. Journal of Life Science 20: 1186-1192.
Kim J H, Kende H. 2004. A transcriptional coactivator, AtGIF1, is involved in regulating leaf growth and morphology in Arabidopsis. Proceedings of the National Academy of Sciences, USA 101: 13374.
Lee B H, Ko J-H, Lee S, Lee Y, Pak J-H, Kim J H. 2009. The Arabidopsis GRF-INTERACTING FACTOR gene family performs an overlapping function in determining organ size as well as multiple developmental properties. Plant Physiology 151: 655.
Li N, Li Y. 2016. Signaling pathways of seed size control in plants. Current Opinion in Plant Biology 33: 23-32.
Li N, Liu Z, Wang Z, Ru L, Gonzalez N, Baekelandt A, Pauwels L, Goossens A, Xu R, Zhu Z et al. 2018a. STERILE APETALA modulates the stability of a repressor protein complex to control organ size in Arabidopsis thaliana. PLoS Genetics 14: e1007218.
Li N, Xu R, Duan P, Li Y. 2018b. Control of grain size in rice. Plant Reproduction 31: 237-251.
Li N, Xu R, Li Y. 2019. Molecular networks of seed size control in plants. Annual Review of Plant Biology 70: 435-463.
Li X, Liu W, Zhuang L, Zhu Y, Wang F, Chen T, Yang J, Ambrose M, Hu Z, Weller J L et al. 2018. BIGGER ORGANS and ELEPHANT EAR-LIKE LEAF1 control organ size and floral organ internal asymmetry in pea. Journal of Experimental Botany 70: 179-191.
Liao B-Y, Weng M-P. 2015. Unraveling the association between mRNA expressions and mutant phenotypes in a genome-wide assessment of mice. Proceedings of the National Academy of Sciences, USA 112: 4707.
Libault M, Thibivilliers S, Bilgin D D, Radwan O, Benitez M, Clough S J, Stacey G. 2008. Identification of four soybean reference genes for gene expression normalization. Plant Genome 1: 44-54.
Liu B, Fujita T, Yan Z-H, Sakamoto S, Xu D, Abe J. 2007. QTL mapping of domestication-related traits in soybean (Glycine max). Annals of Botany 100: 1027-1038.
Liu D, Yan Y, Fujita Y, Xu D. 2018. Identification and validation of QTLs for 100-seed weight using chromosome segment substitution lines in soybean. Breeding Science 68: 442-448.
Liu Y, Li Y, Reif J C, Mette M F, Liu Z, Liu B, Zhang S, Yan L, Chang R, Qiu L. 2013. Identification of quantitative trait loci underlying plant height and seed weight in soybean. Plant Genome 6: 1-11.
Liu Z, Li N, Zhang Y, Li Y. 2020. Transcriptional repression of GIF1 by the KIX-PPD-MYC repressor complex controls seed size in Arabidopsis. Nature Communications 11: 1846.
Lu X, Xiong Q, Cheng T, Li Q-T, Liu X-L, Bi Y-D, Li W, Zhang W-K, Ma B, Lai Y-C et al. 2017. A PP2C-1 allele underlying a quantitative trait locus enhances soybean 100-seed weight. Molecular Plant 10: 670-684.
Moyle R L, Carvalhais L C, Pretorius L-S, Nowak E, Subramaniam G, Dalton-Morgan J, Schenk P M. 2017. An optimized transient dual luciferase assay for quantifying microRNA directed repression of targeted sequences. Frontiers in Plant Science 8: 1631.
Naito K, Takahashi Y, Chaitieng B, Hirano K, Kaga A, Takagi K, Ogiso-Tanaka E, Thavarasook C, Ishimoto M, Tomooka N. 2017. Multiple organ gigantism caused by mutation in VmPPD gene in blackgram (Vigna mungo). Breeding Science 67: 151-158.
Panthee D R, Pantalone V R, West D R, Saxton A M, Sams C E. 2005. Quantitative trait loci for seed protein and oil concentration, and seed size in soybean. Crop Science 45: 2015-2022.
Pillitteri L J, Bemis S M, Shpak E D, Torii K U. 2007. Haploinsufficiency after successive loss of signaling reveals a role for ERECTA-family genes in Arabidopsis ovule development. Development 134: 3099.
Purugganan M D, Fuller D Q. 2009. The nature of selection during plant domestication. Nature 457: 843-848.
Roulin A, Auer P L, Libault M, Schlueter J, Farmer A, May G, Stacey G, Doerge R W, Jackson S A. 2013. The fate of duplicated genes in a polyploid plant genome. The Plant Journal 73: 143-153.
Schmutz J, Cannon S B, Schlueter J, Ma J, Mitros T, Nelson W, Hyten D L, Song Q, Thelen J J, Cheng J et al. 2010. Genome sequence of the palaeopolyploid soybean. Nature 463: 178-183.
Seidman J G, Seidman C. 2002. Transcription factor haploinsufficiency: when half a loaf is not enough. Journal of Clinical Investigation 109: 451-455.
Smith T J, Camper H M. 1975. Effects of seed size on soybean performance. Agronomy Journal 67: 681-684.
Stacey M G, Cahoon R E, Nguyen H T, Cui Y, Sato S, Nguyen C T, Phoka N, Clark K M, Liang Y, Forrester J et al. 2016. Identification of homogentisate dioxygenase as a target for vitamin E biofortification in oilseeds. Plant Physiology 172: 1506.
Stemmer M, Thumberger T, del Sol Keyer M, Wittbrodt J, Mateo J L. 2015. CCTop: an intuitive, flexible and reliable CRISPR/Cas9 target prediction tool. PLoS ONE 10: e0124633.
Swinnen G, Goossens A, Pauwels L. 2016. Lessons from domestication: targeting cis-regulatory elements for crop improvement. Trends in Plant Science 21: 506-515.
Tamura K, Stecher G, Peterson D, Filipski A, Kumar S. 2013. MEGA6: molecular evolutionary genetics analysis version 6.0. Molecular Biology and Evolution 30: 2725-2729.
Teng W, Han Y, Du Y, Sun D, Zhang Z, Qiu L, Sun G, Li W. 2008. QTL analyses of seed weight during the development of soybean (Glycine max L. Merr.). Heredity 102: 372-380.
Van Bel M, Diels T, Vancaester E, Kreft L, Botzki A, Van de Peer Y, Coppens F, Vandepoele K. 2017. PLAZA 4.0: an integrative resource for functional, evolutionary and comparative plant genomics. Nucleic Acids Research 46: D1190-D1196.
Vincent J A, Stacey M, Stacey G, Bilyeu K D. 2015. Phytic acid and inorganic phosphate composition in soybean lines with independent IPK1 mutations. Plant Genome 8: 1-10.
Wang Z, Li N, Jiang S, Gonzalez N, Huang X, Wang Y, Inz'e D, Li Y. 2016. SCFSAP controls organ size by targeting PPD proteins for degradation in Arabidopsis thaliana. Nature Communications 7: 11192.
White D W R. 2006. PEAPOD regulates lamina size and curvature in Arabidopsis. Proceedings of the National Academy of Sciences, USA 103: 13238-13243.
White D W R. 2017. PEAPOD limits developmental plasticity in Arabidopsis.bioRxiv 102707.
Yan L, Hofmann N, Li S, Ferreira M E, Song B, Jiang G, Ren S, Quigley C, Fickus E, Cregan P et al. 2017. Identification of QTL with large effect on seed weight in a selective population of soybean with genome-wide association and fixation index analyses. BMC Genomics 18: 529.
Yuan L, Dou Y, Kianian S F, Zhang C, Holding D R. 2014. Deletion mutagenesis identifies a haploinsufficient role for c-zein in opaque2 endosperm modification. Plant Physiology 164: 119.
Zeng P, Vadnais D A, Zhang Z, Polacco J C. 2004. Refined glufosinate selection in Agrobacterium-mediated transformation of soybean [Glycine max (L.) Merrill]. Plant Cell Reports 22: 478-482.
Zhang J, Song Q, Cregan P B, Jiang G-L. 2016. Genome-wide association study, genomic prediction and marker-assisted selection for seed weight in soybean (Glycine max). Theoretical and Applied Genetics 129: 117-130.
Zhang Y, Du L, Xu R, Cui R, Hao J, Sun C, Li Y. 2015. Transcription factors SOD7/NGAL2 and DPA4/NGAL3 act redundantly to regulate seed size by directly repressing KLU expression in Arabidopsis thaliana. Plant Cell 27: 620.
Zhang Y, Li W, Lin Y, Zhang L, Wang C, Xu R. 2018. Construction of a high-density genetic map and mapping of QTLs for soybean (Glycine max) agronomic and seed quality traits by specific length amplified fragment sequencing. BMC Genomics 19: 641.
Zhao X, Dong H, Chang H, Zhao J, Teng W, Qiu L, Li W, Han Y. 2019. Genome wide association mapping and candidate gene analysis for hundred seed weight in soybean [Glycine max (L.) Merrill]. BMC Genomics 20: 648.
Zhou D-X. 1999. Regulatory mechanism of plant gene transcription by GT-elements and GT-factors. Trends in Plant Science 4: 210-214.
Zhou Z, Jiang Y, Wang Z, Gou Z, Lyu J, Li W, Yu Y, Shu L, Zhao Y, Ma Y et al. 2015. Resequencing 302 wild and cultivated accessions identifies genes related to domestication and improvement in soybean. Nature Biotechnology 33: 408-414.
Zhu Y, Luo X, Liu X, Wu W, Cui X, He Y, Huang J. 2020. Arabidopsis PEAPODs function with LIKE HETEROCHROMATIN PROTEIN1 to regulate lateral organ growth. Journal of Integrative Plant Biology 62: 812-831.

1-16. (canceled)

17. A method for producing a soybean plant comprising in its genome an introgressed genetic locus comprising a genotype associated with a large-seed phenotype, the method comprising the steps of:
a. crossing a first soybean plant with a genotype associated with a large-seed phenotype in a first polymorphic genetic locus comprising the soybean GmKIX8-1 gene Glyma.17G112800 with a second soybean plant comprising a genotype not associated with a large-seed phenotype in the polymorphic genetic locus comprising Glyma.17G112800 and at least one second polymorphic locus that is linked to the genetic locus comprising Glyma.17G112800 and that is not present in said first soybean plant to obtain a population segregating for the large-seed phenotype polymorphic locus and said linked second polymorphic locus;
b. genotyping for the presence of at least two polymorphic nucleic acids in at least one soybean plant from said population, wherein a first polymorphic nucleic acid is located in said genetic locus comprising Glyma.17G112800 and wherein a second polymorphic amino acid is the linked second polymorphic locus not present in said first soybean plant; and
c. selecting a soybean plant comprising a genotype associated with the large-seed phenotype and the at least one linked marker found in said second soybean plant that does not comprise a large-seed phenotype locus but not found in said first soybean plant, thereby obtaining a soybean plant comprising in its genome an introgressed large-seed phenotype locus.

18. The method for producing a soybean plant of claim 17, wherein the genetic locus comprising a genotype associated with a large-seed phenotype is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the GmKIX8-1 gene Glyma.17G112800.

19. The method for producing a soybean plant of claim 17, wherein the genetic locus comprising a genotype associated with a large-seed phenotype consists of the GmKIX8-1 gene Glyma.17G112800.

20. The method for producing a soybean plant of claim 17, wherein the genotype associated with the large-seed phenotype is a deletion of or within the GmKIX8-1 gene Glyma.17G112800.

21. The method for producing a soybean plant of claim 20, wherein the genotype associated with the large-seed phenotype is a deletion within the protein coding region and/or promoter region of the GmKIX8-1 gene Glyma.17G112800,
optionally, (i) wherein said promoter region deletion comprises a deletion of the tandem repeated sequence CGC located at −177 to −174 relative to the GmKIX8-1 gene ATG start site;
optionally, (ii) wherein said promoter region deletion comprises a deletion of the tandem repeated sequence GT located at −104 to −92 relative to the GmKIX8-1 gene ATG start site; or
optionally, wherein said promoter region deletion comprises both the deletions of (i) and (ii).

22-24. (canceled)

25. The method for producing a soybean plant of claim 17, wherein the population of soybean plants produced comprises a plurality of soybean plants that exhibit the large-seed phenotype.

26. The method for producing a soybean plant of claim 17, wherein said second linked polymorphic locus is detected with a marker that is located within about 1 Mb, 500, 100, 40, 20, 10, or 5 kilobases (Kb) of Glyma.17G112800.

27-28. (canceled)

29. A soybean plant comprising an introgressed genetic locus comprising a genotype associated with a large-seed phenotype in a genomic region comprising the soybean GmKIX8-1 gene Glyma.17G112800I, wherein at least one marker linked to the introgressed large-seed phenotype genetic locus found in said soybean plant is characteristic of germplasm comprising a non-large-seed genetic locus but is not associated with germplasm comprising the large-seed phenotype genetic locus.

30. The soybean plant of claim 29, wherein the genetic locus is a genomic region between any of about 1 Mb, 500 kb, 100 kb, 50 kb, or 10 kb telomere proximal and any of about 1 Mb, 500 kb, 100 kb, 50 kb, or 10 kb centromere proximal of the GmKIX8-1 gene Glyma.17G112800I.

31. The soybean plant of claim 29, wherein the genetic locus consists of the GmKIX8-1 gene Glyma.17G112800I.

32. The soybean plant of claim 29, wherein the genotype associated with the large-seed phenotype is a deletion of or within the GmKIX8-1 gene Glyma.17G112800.

33. The soybean plant of claim 32, wherein the genotype associated with the large-seed phenotype is a deletion within the protein coding region and/or promoter region of the GmKIX8-1 gene Glyma.17G112800,
optionally, (i) wherein said promoter region deletion comprises a deletion of the tandem repeated sequence CGC located at −177 to −174 relative to the GmKIX8-1 gene ATG start site;
optionally, (ii) wherein said promoter region deletion comprises a deletion of the tandem repeated sequence GT located at −104 to −92 relative to the GmKIX8-1 gene ATG start site; or
optionally, wherein said promoter region deletion comprises both the deletions of (i) and (ii).

34-36. (canceled)

37. The soybean plant of claim 29, wherein the soybean plant exhibits the large-seed phenotype.

38. The soybean plant of claim 29, wherein the genetic locus comprising the GmKIX8-1 gene Glyma.17G112800I having a genotype associated with a large-seed phenotype has been gene edited.

39-51. (canceled)

52. An edited soybean GmKIX8-1 gene comprising:
(i) a variant polynucleotide comprising a loss-of-function GmKIX8-1 gene variant, wherein the variant polynucleotide comprises at least one nucleotide insertion, deletion, and/or substitution in comparison to the corresponding unedited wild-type polynucleotide sequence, and
wherein the variant polynucleotide exhibits reduced or loss of expression of the GmKIX8-1 gene in comparison to the wild-type polynucleotide sequence and/or encodes a GmKIX8-1 protein variant having reduced activity in comparison to wild-type GmKIX8-1 protein,
(ii) a variant polynucleotide encoding a loss-of-function GmKIX8-1 protein variant or fragment thereof,
wherein the variant polynucleotide comprises at least one nucleotide insertion, deletion, and/or substitution in comparison to the corresponding unedited wild-type polynucleotide sequence and does not encode for the wild-type GmKIX8-1 protein, and
wherein the variant polynucleotide encodes for a GmKIX8-1 protein variant having reduced activity in comparison to wild-type GmKIX8-1 protein,
optionally, wherein said variant polynucleotide is operably linked to a polynucleotide comprising a promoter;
(iii) a variant polynucleotide comprising a GmKIX8-1 gene 3′ UTR,
wherein the variant polynucleotide comprises at least one nucleotide insertion, deletion, and/or substitution in the 3′ UTR in comparison to the corresponding unedited wild-type polynucleotide sequence, and
wherein the variant 3′ UTR results in reduced or loss of expression of the GmKIX8-1 gene in comparison to the wild-type polynucleotide sequence,
optionally wherein said variant polynucleotide is operably linked to a polynucleotide comprising a promoter and/or a GmKIX8-1 protein coding region;
(iv) a variant polynucleotide comprising a GmKIX8-1 gene 5′ UTR,
wherein the variant polynucleotide comprises at least one nucleotide insertion, deletion, and/or substitution in the 5′ UTR in comparison to the corresponding unedited wild-type polynucleotide sequence, and
wherein the variant 5′ UTR results in reduced or loss of expression of the GmKIX8-1 gene in comparison to the wild-type polynucleotide sequence,
optionally wherein said variant polynucleotide is operably linked to a polynucleotide comprising a promoter and/or a GmKIX8-1 protein coding region;
(v) a variant polynucleotide comprising a GmKIX8-1 gene promoter,
wherein the variant polynucleotide comprises at least one nucleotide insertion, deletion, and/or substitution in the promoter in comparison to the corresponding unedited wild-type polynucleotide sequence, and
wherein the variant promoter results in reduced or loss of expression of the GmKIX8-1 gene in comparison to the wild-type polynucleotide sequence,
optionally, wherein said variant polynucleotide is operably linked to a polynucleotide comprising a GmKIX8-1 protein coding region;
(vi) a variant polynucleotide comprising a GmKIX8-1 gene intron,
wherein the variant polynucleotide comprises at least one nucleotide insertion, deletion, and/or substitution in the intron in comparison to the corresponding unedited wild-type polynucleotide sequence, and
wherein the variant intron results in reduced or loss of expression of the GmKIX8-1 gene in comparison to the wild-type polynucleotide sequence,
optionally, wherein said variant polynucleotide is operably linked to a polynucleotide comprising at least one GmKIX8-1 gene exon; and/or
(vii) a variant polynucleotide comprising a GmKIX8-1 gene exon,
wherein the variant polypeptide comprises at least one nucleotide insertion, deletion, and/or substitution in the exon in comparison to the corresponding unedited wild-type polynucleotide sequence, and
wherein the variant exon results in reduced or loss of expression of the GmKIX8-1 gene in comparison to the wild-type polynucleotide sequence and/or encodes a GmKIX8-1 protein variant having reduced activity in comparison to wild-type GmKIX8-1 protein,
optionally wherein said variant polynucleotide is operably linked to a polynucleotide comprising a promoter and/or a GmKIX8-1 gene intron, and
optionally, wherein the variant polypeptide comprises a deletion of at least a portion of exon 1, exon 2, or both exon 1 and exon 2 of the GmKIX8-1 gene.

53. The edited soybean GmKIX8-1 gene of claim 52 comprising a variant polypeptide comprising a loss-of-function GmKIX8-1 gene variant,
wherein the variant polynucleotide comprises a deletion of a portion of the 3′ end of exon 1 of the GmKIX8-1 gene, a deletion of the intron between exon 1 and exon 2 of the GmKIX8-1 gene, and a deletion of a portion the 5′ end of exon 2 of the GmKIX8-1 gene in comparison to the corresponding unedited wild-type polynucleotide sequence.

54. The edited soybean GmKIX8-1 gene of claim 52 wherein the variant polynucleotide comprises at least one nucleotide deletion in the promoter in comparison to the corresponding unedited wild-type polynucleotide sequence, and
wherein the nucleotide deletion results in reduced or loss of expression of the GmKIX8-1 gene in comparison to the corresponding wild-type polynucleotide sequence.

55. The edited soybean GmKIX8-1 gene of claim 52 comprising a variant polynucleotide encoding a loss-of-function GmKIX8-1 protein variant or fragment thereof,
wherein the variant polynucleotide comprises at least one nucleotide insertion, deletion, and/or substitution in comparison to the corresponding unedited wild-type polynucleotide sequence and does not encode for the wild-type GmKIX8-1 protein, and
wherein the variant polynucleotide encodes for a GmKIX8-1 protein variant having reduced activity in comparison to wild-type GmKIX8-1 protein.

56. (canceled)

57. A plant nuclear genome comprising the edited soybean GmKIX8-1 gene of claim 52;
optionally, wherein the variant polynucleotide is heterologous to the nuclear genome.

58. The plant nuclear genome of claim 57, wherein said variant polynucleotide is operably linked to an endogenous promoter of the nuclear genome.

59-90. (canceled)
